(19) United States
(12) Patent Application Publication
(10) Pub. No.: US 2025/0332415 A1
(43) Pub. Date: Oct. 30, 2025

(54) PARAMETER VARIATIONS IN NEURAL STIMULATION

(71) Applicant: Cala Health, Inc., San Mateo, CA (US)

(72) Inventors: Alexander R. Kent, Mountain View, CA (US); Gregory T. Schulte, Oakland, CA (US); Shengzhi Li, Mountain View, CA (US); Jessica M. Liberatore, San Mateo, CA (US); Samuel Richard Hamner, San Francisco, CA (US); Mark Shughart, Redwood City, CA (US); Musa Ozturk, Belmont, CA (US); Kathryn H. Rosenbluth, San Francisco, CA (US)

(21) Appl. No.: 18/294,493

(22) PCT Filed: Aug. 1, 2022
(86) PCT No.: PCT/US2022/074377

§ 371 Date: Feb. 1, 2024

Related U.S. Application Data
(60) Provisional application No. 63/203,895, filed on Aug. 3, 2021.
(60) Provisional application No. 63/264,498, filed on Nov. 23, 2021.

(51) Int. Cl. A61N 1/36; A61N 1/04

(52) U.S. Cl. CPC A61N 1/36034; A61N 1/0456; A61N 1/0476; A61N 1/36003

(57) ABSTRACT

Disclosed herein are systems, devices, and methods for stimulating nerves, including electrically stimulating peripheral nerve(s) to treat various diseases and disorders, as well as systems and methods for applying stimulation waveforms for improving the therapeutic benefit, outcomes, and/or experience relating to the same. Some systems and methods generate a biphasic stimulation waveform that includes a plurality of pulses with alternating leading phases. Some systems and methods vary one or more parameters of the stimulation waveform to correlate with a characteristic of the user. Example characteristic of the user include a phase of a tremor exhibited by the user and a respiratory cycle of the user. For some systems and methods, the stimulation waveform includes a plurality of burst with each burst ramping up in amplitude. For some systems and methods, the stimulation waveform is delivered during a time period that includes an on-cycle portion and an off-cycle portion with the stimulation waveform only being delivered during the on-cycle portion of the time period.

### REFERENCE TO RELATED APPLICATIONS

This application claims the benefit of U.S. Provisional Application Nos. 63/203,895 and 63/264,498, filed Aug. 3, 2021 and Nov. 23, 2021, respectively, the entire disclosure of each is hereby incorporated by reference in its entirety.

### TECHNICAL FIELD

Some embodiments of the invention relate generally to systems, devices, and methods for neuromodulating (such as stimulating) nerves, and more specifically relate to systems, devices, and methods for electrically stimulating peripheral nerve(s) to treat disorders and/or associated symptoms, as well as systems and methods for applying stimulation waveforms for improving the therapeutic benefit, outcomes, and/or experience relating to the same.

### BACKGROUND

Electrical energy can be delivered transcutaneously via electrodes on the skin surface with neurostimulation systems to stimulate peripheral nerves. Essential tremor is a common movement disorder, with growing numbers due to the aging population. Tremor in the hands and forearm is especially prevalent and problematic because it makes it difficult to write, type, eat, and drink. Disorders, including essential tremor, may be treated by pharmaceutical agents, which can cause undesired side effects. Applicant's own work has demonstrated that electrical energy can be delivered transcutaneous via electrodes on the skin surface with neurostimulation systems to stimulate peripheral nerves, including on a patient's limb. For example, Applicant's prior treatment of tremor and other disorders has been effective in many cases (see, for example, U.S. Pat. No. 9,452,287).

### SUMMARY

Wearable systems with compact, ergonomic form factors are needed to enhance efficacy, compliance, and comfort with using the devices. Embodiments of the system disclosed herein provides automatic amplitude adjustment to neuromodulation signals over time. The system can employ machine learning to adjust the signals used to stimulate, for example, the median, ulnar and radial nerves. For example, during therapy the stimulation of both nerves can be dynamic which maintains a level of patient comfort during the treatment. In embodiments, the machine learning algorithm determines a relative percentage of amplitude across all affected nerves so that the patient only sets the amplitude for one of the nerve targets. Embodiments of the system train the machine learning algorithm by evaluating anatomical stimulation studies where typical offsets would be initially derived. The initial preset created by the patient during set up would allow the algorithm to personalize the offsets. Embodiments of the system allow other inputs to the machine learning algorithm including demographics (age, sex, wrist size, body-mass index, race, ethnicity, etc.), medical history (ET medications, years since initially ET diagnosis, etc.), tremor characteristics (tremor frequency, tremor amplitude, etc.). Embodiments of the system employ additional sensors to allow automatic adjustment of one or both amplitudes. For example, embodiments of the system can employ a normal force transducer to measure tightness of the band on the wrist and/or a sensor that automatically measures the wrist circumference of the patient. Any combination of these measurements and machine learning allows the device to initially set and automatically adjusted the amplitude throughout therapy without active intervention by the patient.

Some embodiments of the system activate nerves in a different way anatomically to increase variability in the areas of the nerves. Embodiments of the system employ multiple channels to activate different areas and create a more durable benefit for the patient. For example, the system can leverage “uncoupled commons”, “coupled commons”, and “opposite commons”, to increase the potential number of channels through the wrist and generate different individual and combinations of nerve stimulation. These channels are then used in an algorithm to maximize variability in nerve activation (e.g., activation of different nerve branches). Embodiments of the system can (1) alternate between different channels during all therapy sessions; (2) employ an initial “run in period” to determine a typical placement, stimulation amplitude, alternating patterns of stimulation, and improvement over a series of sessions to then prescribe an appropriate nerve patterning that is the most effective per user; and/or (3) employ preset patterns that are updated by the patient, or adjusted via algorithms that can be turned on/off as desired.

Some embodiments of the system can take into account difference between each patient's nerve anatomy as well as how the specific nerve branches are activated with the stimulation channels. Embodiments of the system can combine anatomical jitter with stimulation amplitude jitter (periodic adjustment in stimulation amplitude) to produce even greater variability in nerve activation.

Some embodiments of the system can leverage multiple elements to vary therapy to prevent habituation and/or adjust amplitude to manage discomfort. Embodiments of the system can employ a therapeutic kick-start to raise the amplitude high for a period of time to promote a patient's therapeutic response. After the patient's response is started, a low-level amplitude can maintain their therapy. Embodiments of the system can provide (1) a periodic therapeutic kick-start which delivers a maximum tolerance preset multiple times throughout a therapy session to prevent habituation; (2) an adaptive waveform over therapy which leverages the slope of tremor-session time data to change the stimulation amplitude; and/or (3) independent amplitude configurations which allow the amplitude to be adjusted independently to activate different nerves.

Some embodiments of the system can automatically adjust a stimulation intensity based on the real-time measured system impedance to maintain a consistent level of paresthesia sensations over time. The patient can set the initial stimulation current amplitude, and then the system can automatically increase or decrease the current based on the measured impedance. With this adjustment, a consistent level of paresthesia sensations is maintained over time despite any changes in the electrode-skin contact. Embodiments of the system can be trained on (1) data collected during a patient's initial stimulation session and/or (2) patient-selected amplitudes and measured impedances during each session. Embodiments of the system employ an algorithm which establishes an optimal relationship between stimulation intensity and impedance. The algorithm can use simple regression analysis or machine learning approaches to automatically adjust stimulation amplitude.

Some embodiments of the neurostimulation system disclosed herein can deliver stimulation pulses with alternating leading phases (e.g., a cathodic-first followed by an anodic-first phase). By alternating the leading phase, electro-chemical changes in the electrode-skin interface caused by prolonged percutaneous stimulation sessions employing a constant pattern of a leading cathodic or anodic first phase may be reduced. In theory, each pulse of a biphasic waveform (e.g., constant cathodic-first or constant anodic-first phase) is charge balanced due to the current flowing in both directions during the pulse. In practice, biphasic operation still results in electro-chemical changes in the electrode-skin interface due to current flowing across the skin causing discomfort and adverse biological effects (e.g., skin irritations). In some embodiments, alternating the leading phase of at least some pulses within the stimulation session can mitigate against such adverse biological effects.

Some embodiments of the neurostimulation system disclosed herein can deliver stimulation pulses based at least in part on measured, real-time phases of the patient's tremor. For example, the system can deliver a pulse to a first nerve while the tremor is between phases 0-180 degrees (e.g., hand moving in downward direction) and then deliver a pulse to a second nerve when the tremor is between phases 180-360 degrees (e.g., hand moving in upward direction). Phase-locked stimulation may increase tremor reduction by shifting pathological oscillations away from their peak resonance frequency and/or enhancing spike-timing-dependent plasticity (STDP).

Some embodiments of the neurostimulation system disclosed herein can deliver stimulation pulses based at least in part on the patient's respiratory cycle (e.g., respiratory gating). For example, in some embodiments, the timing of median and radial (or ulnar) nerve stimulation can be determined based on the measured, real-time phases of the respiratory cycle. Delivering stimulation at a specific phase of the respiration cycle may enhance autonomic modulatory effects. In some embodiments, a first target nerve can be modulated during an inspiratory phase of the respiratory cycle, and a second target nerve can be modulated during an expiratory phase of the respiratory cycle. In this way, the device can be configured to synchronize/gate the stimulation to one or more particular phases of the respiratory cycle. In some embodiments, the device can identify specific points on the respiratory signal that may be more receptive to stimulation. In some embodiments, the stimulation can be synchronized to early expiration, late expiration, early inspiration, and/or late inspiration.

Some embodiments of the neurostimulation system disclosed herein can deliver stimulation pulses by ramping up the current amplitude during a burst, after a prespecified time period, or after a prespecified number of bursts. For example, a range of the current amplitude can correspond to the patient's minimum sensory threshold or paresthesia threshold (lower bound) and a maximum comfortable threshold (upper bound). Higher stimulation amplitudes may activate smaller and more distant neural fibers in the median and radial (or ulnar) nerves. Dynamically changing the amplitude within a burst may generate asynchronous and stochastic activation that is distributed across nerve fibers. This activation may enhance the therapeutic mechanism of action by increasing desynchronization in downstream brain targets. The dynamic changes in amplitude may also generate more natural-feeling paresthesia sensations.

Some embodiments of the neurostimulation system disclosed herein accommodate variability in pathological tremor characteristics including variations in tremor pathology for a user. For example, the frequency of a tremor experienced by the user is not constant over time. The neurostimulation system can deliver a stimulation waveform that varies one or more parameters, as opposed to delivering a constant value, to improve the therapeutic response of the stimulation. For example, adding variation in burst frequency may account for natural variation in pathological tremor frequency. In some cases, pathological tremor frequency can change, for example, by more than 2 Hz between tasks and by up to 32% on the same task over time within an individual subject. Calibrating burst frequency to tremor frequency can improve therapeutic effect.

In certain embodiments, stimulation parameters are agnostic for any particular individual and may be varied within generally known therapeutic ranges during the course of stimulation. Adding variation in pulse frequency may account for individual differences in the brain response to peripheral nerve stimulation. For example, the evoked response generated in the ventral intermediate nucleus of the thalamus by median nerve stimulation was maximized at a pulse frequency of 50 Hz in some subjects and 100 Hz in other subjects. By varying pulse frequency throughout these range of values, the brain response is maximized during some portion of the therapy session for every individual, which may enhance therapeutic benefit.

In various embodiments, one or more of the following nerves are treated such as the median, radial, and/or ulnar nerves in the upper extremities, tibial, saphenous, and/or peroneal nerve in the lower extremities; or the auriculotemporal, great auricular, vagus, trigeminal or cranial nerves on the head or ear, as non-limiting examples. In some embodiments, the median nerve is modulated (e.g., stimulated) along with one, two or more other nerves. Stimulation of these nerves, according to several embodiments described herein, are used to treat essential tremor, Parkinson's tremor, orthostatic tremor, and multiple sclerosis, urological disorders, gastrointestinal disorders, cardiac diseases, and mood disorders (including but not limited to depression, bipolar disorder, dysthymia, and anxiety disorder), pain syndromes (including but not limited to migraines and other headaches, trigeminal neuralgia, fibromyalgia, complex regional pain syndrome), Lyme disease, stroke, among others. Inflammatory bowel disease (such as Crohn's disease), rheumatoid arthritis, multiple sclerosis, psoriatic arthritis, psoriasis, chronic fatigue syndrome, and other inflammatory diseases are treated in several embodiments. Cardiac conditions (such as atrial fibrillation, hypertension, and stroke) are treated in one embodiment. Epilepsy is treated in one embodiment. Inflammatory skin conditions and immune dysfunction are also treated in some embodiments.

Several embodiments comprise a device such as a wearable neurostimulation device for providing variability in stimulation. In one embodiment, the device comprises a non-implantable band configured to at least partially encircle a limb (such as the wrist, upper arm, ankle, upper leg, etc.) of a user. The device, such as the band, also comprises in one embodiment a first stimulating electrode positioned on the band to target a first peripheral nerve of the user, a second stimulating electrode positioned on the band to target a second peripheral nerve of the user, one or more common electrodes adjacent the first peripheral nerve, and one or more common electrodes adjacent the second peripheral nerve. The device also comprises in one embodiment one or more processors (such as hardware processors) configured to create a first stimulation channel between the first stimulating electrode and one or more common electrodes adjacent the second peripheral nerve and create a second stimulation channel between the second stimulating electrode and the one or more common electrodes adjacent the first peripheral nerve. Variability in stimulation is provided by delivering a first and second stimulation signal (during for example, a first and second time frame). In some embodiments, there are 2-4 common electrodes. In some embodiments, a third, fourth, fifth (or more) stimulating electrodes are provided. The first stimulation channel is optionally and additionally formed between the first stimulating electrode and one or more common electrodes adjacent the first peripheral nerve. The second stimulation channel can further comprise one or more common electrodes adjacent the second peripheral nerve. The variability between stimulation current paths of the first stimulation channel and the second stimulation channel can activate different nerve branches of the first and second peripheral nerves. Such variability can result in reduced habituation, increased efficacy and a more durable effect.

In some embodiments, a wearable neurostimulation device for transcutaneously stimulating one or more peripheral nerves of a user is provided. In some embodiments, the device comprises a band configured to encircle a limb of the user, a first stimulating electrode positioned on the band to target a first peripheral nerve of the user, one or more first common electrodes positioned on the band adjacent to the first stimulating electrode, a second stimulating electrode positioned on the band to target a second peripheral nerve of the user, one or more second common electrodes positioned on the band adjacent to the second stimulating electrode, and one or more hardware processors configured to: determine a first stimulation channel between the first stimulating electrode and the one or more second common electrodes during a first time frame and determine a second stimulation channel between the second stimulating electrode and the one or more first common electrodes during a second time frame.

In some embodiments, a wearable neurostimulation device for transcutaneously stimulating one or more peripheral nerves of a user is provided. In some embodiments, the device comprises a band configured to encircle a limb of the user, a first stimulating electrode positioned on the band to target a first peripheral nerve of the user, one or more first common electrodes positioned on the band adjacent to the first stimulating electrode, a second stimulating electrode positioned on the band to target a second peripheral nerve of the user, one or more second common electrodes positioned on the band adjacent to the second stimulating electrode, and one or more hardware processors configured to: determine a first stimulation channel between the first stimulating electrode, the one or more first common electrodes, and the one or more second common electrodes during a first time frame and determine a second stimulation channel between the second stimulating electrode, the one or more second common electrodes, and the one or more first common electrodes during a second time frame.

In some embodiments, a wearable neurostimulation device for transcutaneously stimulating one or more peripheral nerves of a user is provided. In some embodiments, the device comprises a band configured to encircle a limb of the user, a stimulating electrode positioned on the band for stimulating a primary target nerve and a secondary target nerve of the user, one or more first common electrodes positioned on the band adjacent to the primary target nerve, one or more second common electrodes positioned on the band adjacent to the secondary target nerve, and one or more hardware processors configured to: determine a first stimulation channel between the first stimulating electrode and the one or more second common electrodes during a first time frame and determine a second stimulation channel between the first stimulating electrode, the one or more second common electrodes, and the one or more first common electrodes during a second time frame.

In some embodiments, a wearable neurostimulation device for transcutaneously stimulating one or more peripheral nerves of a user is provided. In some embodiments, the device comprises a band configured to encircle a limb of the user, a stimulating electrode positioned on the band for stimulating a first peripheral nerve and a second peripheral nerve of the user, one or more first common electrodes positioned on the band adjacent to the first peripheral nerve, one or more second common electrodes positioned on the band adjacent to the second peripheral nerve, and one or more hardware processors configured to: determine a first stimulation channel between the first stimulating electrode, the one or more second common electrodes, and the one or more first common electrodes during a first time frame and determine a second stimulation channel between the first stimulating electrode and the one or more second common electrodes during a second time frame.

In some embodiments, a wearable neurostimulation device for transcutaneously stimulating one or more peripheral nerves of a user is provided. In some embodiments, the device comprises a band configured to encircle a limb of the user, a first peripheral nerve electrode supported by the band and configured to be positioned to deliver stimulation to a first peripheral nerve, a second peripheral nerve electrode supported by the band and configured to be positioned to deliver stimulation to a second peripheral nerve, one or more hardware processors configured to: determine an offset across the first peripheral nerve and the second peripheral nerve, deliver a first stimulation signal to the first peripheral nerve for a prespecified amount of time at a first amplitude, and deliver a second stimulation signal to the second peripheral nerve for a prespecified amount of time at a second amplitude, the second amplitude being determine at least in part based on the offset.

In some embodiments, a wearable neurostimulation device for transcutaneously stimulating one or more peripheral nerves of a user is provided. In some embodiments, the device comprises a band configured to encircle a limb of the user, a peripheral nerve electrode supported by the band and configured to be positioned to deliver stimulation to a peripheral nerve, one or more hardware processors configured to: determine a kick-start stimulation amplitude, deliver a first stimulation signal to the peripheral nerve for a prespecified amount of time at a first amplitude, and deliver a second stimulation signal to the peripheral nerve for a prespecified amount of time at a second amplitude, the second amplitude being determine at least in part based on the kick-start stimulation amplitude.

In some embodiments, a wearable neurostimulation device for transcutaneously stimulating one or more peripheral nerves of a user is provided. In some embodiments, the device comprises a band configured to encircle a limb of the user, a peripheral nerve electrode configured to be positioned against skin of the user to deliver stimulation to a peripheral nerve, one or more hardware processors configured to: determine an impedance of the peripheral nerve electrode; deliver a first stimulation signal to the peripheral nerve for a prespecified amount of time at a first amplitude; and deliver a second stimulation signal to the peripheral nerve for a prespecified amount of time at a second amplitude, the second amplitude being determined at least in part based on the impedance.

In some embodiments, a system for transcutaneously stimulating one or more peripheral nerves of a user is provided. In some embodiments, the system comprises a neurostimulation device and a band configured to encircle a limb of the user. In some embodiments, the band can support a first stimulating electrode positioned on the band to target a first peripheral nerve of the user, a second stimulating electrode positioned on the band to target a second peripheral nerve of the user, one or more first common electrodes positioned on the band adjacent to the first stimulating electrode, and one or more second common electrodes positioned on the band adjacent to the second stimulating electrode, the first stimulating electrode and the one or more second common electrodes being arranged and configured to determine a first stimulation channel for delivering electrical stimuli from the neurostimulation device to the first peripheral nerve during a first time frame, the second stimulating electrode and the one or more first common electrodes being arranged and configured to determine a second stimulation channel for delivering electrical stimuli from the neurostimulation device to the second peripheral nerve during a second time frame.

In some embodiments, a system for transcutaneously stimulating one or more peripheral nerves of a user is provided. In some embodiments, the system comprises a neurostimulation device and a band configured to encircle a limb of the user. In some embodiments, the band can support a first stimulating electrode positioned on the band to target a first peripheral nerve of the user, a second stimulating electrode positioned on the band to target a second peripheral nerve of the user, one or more first common electrodes positioned on the band adjacent to the first stimulating electrode, and one or more second common electrodes positioned on the band adjacent to the second stimulating electrode, the first stimulating electrode, the one or more first common electrodes, and the one or more second common electrodes being arranged and configured to determine a first stimulation channel for delivering electrical stimuli from the neurostimulation device to the first peripheral nerve during a first time frame, the second stimulating electrode, the one or more second common electrodes, and the one or more first common electrodes being arranged and configured to determine a second stimulation channel for delivering electrical stimuli from the neurostimulation device to the second peripheral nerve during a second time frame.

In some embodiments, a system for transcutaneously stimulating one or more peripheral nerves of a user is provided. In some embodiments, the system comprises a neurostimulation device and a band configured to encircle a limb of the user. In some embodiments, the band can support a first stimulating electrode positioned on the band to target a first peripheral nerve and a second peripheral nerve of the user, one or more first common electrodes positioned on the band adjacent to the first peripheral nerve, and one or more second common electrodes positioned on the band adjacent to the second peripheral nerve, the first stimulating electrode and the one or more second common electrodes being arranged and configured to determine a first stimulation channel for delivering electrical stimuli from the neurostimulation device to at least the first peripheral nerve during a first time frame, the first stimulating electrode, the one or more second common electrodes, and the one or more first common electrodes being arranged and configured to determine a second stimulation channel for delivering electrical stimuli from the neurostimulation device to at least the second peripheral nerve during a second time frame.

In some embodiments, a system for transcutaneously stimulating one or more peripheral nerves of a user is provided. In some embodiments, the system comprises a neurostimulation device and a band configured to encircle a limb of the user. In some embodiments, band can support a first stimulating electrode positioned on the band to target a first peripheral nerve and a second peripheral nerve of the user, one or more first common electrodes positioned on the band adjacent to the first peripheral nerve, and one or more second common electrodes positioned on the band adjacent to the second peripheral nerve, the first stimulating electrode, the one or more first common electrodes, and the one or more second common electrodes being arranged and configured to determine a first stimulation channel for delivering electrical stimuli from the neurostimulation device to at least the first peripheral nerve during a first time frame, the first stimulating electrode and the one or more second common electrodes being arranged and configured to determine a second stimulation channel for delivering electrical stimuli from the neurostimulation device to at least the second peripheral nerve during a second time frame.

In some embodiments, a system for transcutaneously stimulating one or more peripheral nerves of a user is provided. In some embodiments, the system comprises a neurostimulation device and a band configured to encircle a limb of the user. In some embodiments, the band can support a first peripheral nerve electrode positioned on the band to target a first peripheral nerve of the user, and a second peripheral nerve electrode positioned on the band to target a second peripheral nerve of the user, the first peripheral nerve electrode being arranged and configured to deliver a first stimulation signal to the first peripheral nerve for a prespecified amount of time at a first amplitude, the second peripheral nerve electrode being arranged and configured to deliver a second stimulation signal to the second peripheral nerve for a prespecified amount of time at a second amplitude, the second amplitude being determine at least in part based on an offset across the first peripheral nerve and the second peripheral nerve.

In some embodiments, a system for transcutaneously stimulating a peripheral nerve of a user is provided. In some embodiments, the system comprises a neurostimulation device and a band configured to encircle a limb of the user. In some embodiments, the band can support a peripheral nerve electrode positioned on the band to deliver a first stimulation signal to the peripheral nerve for a prespecified amount of time at a first amplitude and to deliver a second stimulation signal to the peripheral nerve for a prespecified amount of time at a second amplitude, the second amplitude being determined at least in part based on a kick-start stimulation amplitude.

In some embodiments, a system for transcutaneously stimulating a peripheral nerve of a user is provided. In some embodiments, the system comprises a neurostimulation device and a band configured to encircle a limb of the user. In some embodiments, the band can support a peripheral nerve electrode positioned on the band to be against the skin of the user and deliver a first stimulation signal to the peripheral nerve for a prespecified amount of time at a first amplitude and to deliver a second stimulation signal to the peripheral nerve for a prespecified amount of time at a second amplitude, the peripheral nerve electrode being associated with an impedance, the second amplitude being determined at least in part based on an impedance.

In some embodiments, a system for transcutaneously stimulating one or more peripheral nerves of a user is provided. In some embodiments, the system comprises a neurostimulation device and a band configured to encircle a limb of the user. In some embodiments, the band can support a first stimulating electrode positioned on the band to deliver electrical stimuli from the neurostimulation device to a first peripheral nerve of the user, a second stimulating electrode positioned on the band to deliver electrical stimuli from the neurostimulation device to a second peripheral nerve of the user, and one or more common electrodes positioned on the band adjacent to at least one of the first or second stimulating electrodes.

In some embodiments, a method of neuromodulating one or more peripheral nerves of a user is provided. In some embodiments, the method comprises providing a band configured to encircle a limb of the user, the band comprising a first stimulating electrode positioned on the band to target a first peripheral nerve of the user, one or more first common electrodes positioned on the band adjacent to the first stimulating electrode, a second stimulating electrode positioned on the band to target a second peripheral nerve of the user, one or more second common electrodes positioned on the band adjacent to the second stimulating electrode, determining a first stimulation channel between the first stimulating electrode and the one or more second common electrodes during a first time frame, determining a second stimulation channel between the second stimulating electrode and the one or more first common electrodes during a second time frame, applying a first stimulation signal to the first stimulation channel during the first time frame, and applying a second stimulation signal to the second stimulation channel during the second time frame.

In some embodiments, a method of neuromodulating one or more peripheral nerves of a user is provided. In some embodiments, the method comprises providing a band configured to encircle a limb of the user, the band comprising a first stimulating electrode positioned on the band to target a first peripheral nerve of the user, one or more first common electrodes positioned on the band adjacent to the first stimulating electrode, a second stimulating electrode positioned on the band to target a second peripheral nerve of the user, and one or more second common electrodes positioned on the band adjacent to the second stimulating electrode, determining a first stimulation channel between the first stimulating electrode, the one or more first common electrodes, and the one or more second common electrodes during a first time frame, determining a second stimulation channel between the second stimulating electrode, the one or more second common electrodes, and the one or more first common electrodes during a second time frame, applying a first stimulation signal to the first stimulation channel during the first time frame, and applying a second stimulation signal to the second stimulation channel during the second time frame.

In some embodiments, a method of neuromodulating one or more peripheral nerves of a user is provided. In some embodiments, the method comprises providing a band configured to encircle a limb of the user, the band comprising a stimulating electrode positioned on the band for stimulating a primary target nerve and a secondary target nerve of the user, one or more first common electrodes positioned on the band adjacent to the primary target nerve, and one or more second common electrodes positioned on the band adjacent to the secondary target nerve, determining a first stimulation channel between the first stimulating electrode and the one or more second common electrodes during a first time frame, determining a second stimulation channel between the first stimulating electrode, the one or more second common electrodes, and the one or more first common electrodes during a second time frame, applying a first stimulation signal to the first stimulation channel during the first time frame, and applying a second stimulation signal to the second stimulation channel during the second time frame.

In some embodiments, disclosed herein is a neuromodulation system to modulate one or more peripheral nerves of an arm, hand, wrist, leg, ankle, foot, head, face, neck or ear. In one embodiment, neuromodulation comprises neuromodulation of a first peripheral nerve, a processor and a memory for storing instructions that, when executed by the processor cause the device to neuromodulate a first peripheral nerve for a prespecified amount of time and vary one or more parameters over a prespecified range of parameters at a prespecified rate of variation. Parameters, include for example, burst frequency, pulse frequency, pulse width, intensity, and/or on/off cycling. Non-implantable stimulation via electrodes in a wearable system is provided in several embodiments. Wearable systems include devices that, for example, are placed on the upper arm, upper leg, wrist, finger, ankle, ear, face and neck.

In some embodiments, disclosed herein is a neurostimulation system to stimulate one or more peripheral nerves of an arm, hand, wrist, leg, ankle, foot, head, face, neck or ear, comprising: a first peripheral nerve electrode configured to be positioned to deliver stimulation to a first peripheral nerve; and a processor and a memory for storing instructions that, when executed by the processor cause the device to: deliver stimulation to a first peripheral nerve for a prespecified amount of time; and vary one or more parameters of the first stimulus over a prespecified range of parameters at a prespecified rate of variation, where the parameters could include burst frequency, pulse frequency, pulse width, intensity, and/or on/off cycling. In some embodiments, the varied parameter is restricted to (e.g., consists essentially of or comprises) burst frequency, the range of parameters is restricted to 3-12 Hz (e.g., 3-5, 5-8, 8-12 Hz, and overlapping ranges therein), and the rate of variation is restricted to (e.g., consists essentially of or comprises) 0.001-100 Hz/s (e.g., 0.001-0.01, 0.01-0.1, 0.1-1, 1-10, 10-100 Hz, and overlapping ranges therein). In some embodiments, the varied parameter is restricted to pulse frequency, the range of parameters is restricted to (e.g., consists essentially of or comprises) 50-150 Hz (e.g., 50-100, 100-150 Hz, and overlapping ranges therein), and the rate of variation is restricted to (e.g., consists essentially of or comprises) 0.001-10,000 Hz/s (e.g., 0.001-0.01, 0.01-0.1, 0.1-1, 1-10, 10-100, 100-1,000, 1,000-10,000 Hz/s). In some embodiments, the varied parameter is restricted to (e.g., consists essentially of or comprises) pulse width, the range of parameters is restricted to (e.g., consists essentially of or comprises) a minimum value from one of 100, 150, 200, 250, 300, or 350 microseconds and a maximum pulse width based on an individual's comfort level at a fixed stimulation amplitude, and the rate of variation is restricted to (e.g., consists essentially of or comprises) 0.01-10,000 microseconds per second (e.g., 0.01-0.1, 0.1-1, 1-10, 10-100, 100-1,0000, 1,000-10,000 microseconds per second, and overlapping ranges therein). In some embodiments, the fixed stimulation amplitude is based on an individual's sensory level with a fixed pulse width in a range between 100-500 microseconds (e.g., 100-200, 200-300, 300-400, 400-500 microseconds, and overlapping ranges therein).

In some embodiments, the varied parameter is restricted to (e.g., consists essentially of or comprises) stimulation amplitude, the range of parameters is restricted to (e.g., consists essentially of or comprises) a minimum set to the stimulation amplitude at an individual's minimum sensory threshold and a maximum set to the stimulation amplitude at an individual's maximum comfort level, and the rate of variation is restricted to (e.g., consists essentially of or comprises) 0.001-100 mA/s (e.g., 0.001-0.01, 0.01-0.1, 0.1-1, 1-10 mA/s, and overlapping ranges therein). In some embodiments, the varied parameter is restricted to (e.g., consists essentially of or comprises) stimulation amplitude, the range of parameters is restricted to (e.g., consists essentially of or comprises) a minimum set to a stimulation amplitude at a pre-specified increment below an individual's minimum sensory threshold (sub-sensory) and a maximum set to the stimulation amplitude at an individual's maximum comfort level and the rate of variation is restricted to (e.g., consists essentially of or comprises) 0.001-100 mA/s. In some embodiments, the pre-specified increment is one of 0.1, 0.2, 0.25, 0.3, 0.4, 0.5, 0.6, 0.7, 0.75, 0.8, 0.9 or 1 mA.

In some embodiments, the one or more parameters of the first stimulus comprises a first parameter and a second parameter, and wherein the first parameter and the second parameter are simultaneously varied. For example, the first parameter and the second parameter are alternately varied. In some embodiments, the first parameter and the second parameter are varied on different timescales. In some embodiments, the first parameter and the second parameter are varied based on adaptive learning, and wherein the adaptive learning employs at least one of kinematic measurements or satisfaction data. In other embodiments, combinations of timescales, kinematic data and satisfaction data are used.

In some embodiments, disclosed herein is a neurostimulation system to stimulate one or more peripheral nerves of an arm, hand, wrist, leg, ankle, foot, head, face, neck or ear, comprising: a first peripheral nerve electrode configured to be positioned to deliver stimulation to a first peripheral nerve; a processor and a memory for storing instructions that, when executed by the processor cause the device to: deliver stimulation to a first peripheral nerve for a prespecified amount of time; vary one or more parameters of the first stimulus over a prespecified range of parameter, where the parameters could include burst frequency, pulse frequency, pulse width, intensity, and/or on/off cycling; and/or determine the value of the varied parameter based on a prespecified probabilistic distribution.

In some embodiments, the varied parameter is restricted to (e.g., consists essentially of or comprises) burst frequency, the range of parameters is restricted to (e.g., consists essentially of or comprises) 3-12 Hz (e.g., 3-5, 5-8, 8-12 Hz, and overlapping ranges therein), the rate of variation is restricted to (e.g., consists essentially of or comprises) 0.001-100 Hz/s (e.g., 0.001-0.01, 0.01-0.1, 0.1-1, 1-10, 10-100 Hz, and overlapping ranges therein.

In some embodiments, a neurostimulation system is configured to introduce variability to enhance therapeutic response for a user. In some embodiments, the neurostimulation system comprises a first peripheral nerve electrode configured to be positioned to deliver stimulation to a first peripheral nerve and a processor and a memory for storing instructions that, when executed by the processor cause the system to: generate a stimulation waveform configured to be delivered with the first peripheral nerve electrode for a time period; vary one or more parameters of the stimulation waveform to avoid a constant value for the one or more parameters during the time period; and deliver the generated stimulation waveform to the first peripheral nerve electrode for the time period, wherein the variation in the one or more parameters enhances therapeutic response and/or comfort of stimulation sensations compared to maintaining the one or more parameters constant over the time period.

In some embodiments, a neurostimulation system is configured to introduce variability to enhance therapeutic response for a user. In some embodiments, the neurostimulation system comprises a first peripheral nerve electrode configured to be positioned to deliver stimulation to a first peripheral nerve; and a processor and a memory for storing instructions that, when executed by the processor cause the system to: generate a stimulation waveform configured to be delivered with the first peripheral nerve electrode for a time period; and vary one or more parameters of the stimulation waveform during the time period without probing one or more characteristics of the medical condition with one or more sensors while delivering the stimulation.

In some embodiments, a neurostimulation system is configured to introduce variability to enhance therapeutic response for a user. In some embodiments, the neurostimulation system comprises a first peripheral nerve electrode configured to be positioned to deliver stimulation to a first peripheral nerve; a processor and a memory for storing instructions that, when executed by the processor cause the system to: deliver stimulation to a first peripheral nerve for a prespecified amount of time; and simultaneously vary each of a first parameter and a second parameter of the delivered stimulation over a prespecified range at a prespecified rate of variation.

In some embodiments, a neurostimulation system is configured to introduce variability to enhance therapeutic response for a user. In some embodiments, the neurostimulation system comprises a first peripheral nerve electrode configured to be positioned to deliver stimulation to a first peripheral nerve; a processor and a memory for storing instructions that, when executed by the processor cause the system to: deliver stimulation to a first peripheral nerve for a prespecified amount of time; and alternately vary in a braided manner each of a first parameter and a second parameter of the delivered stimulation over a prespecified range at a prespecified rate of variation.

In some embodiments, a neurostimulation system is configured to introduce variability to enhance therapeutic response for a user. In some embodiments, the neurostimulation system comprises a first peripheral nerve electrode configured to be positioned to deliver stimulation to a first peripheral nerve; a processor and a memory for storing instructions that, when executed by the processor cause the system to: deliver stimulation to a first peripheral nerve for a prespecified amount of time; and vary each of a first parameter and a second parameter of the delivered stimulation on different timescales over a prespecified range at a prespecified rate of variation.

In some embodiments, a neurostimulation system is configured to introduce variability to enhance therapeutic response for a user. In some embodiments, the neurostimulation system comprises a first peripheral nerve electrode configured to be positioned to deliver stimulation to a first peripheral nerve; a processor and a memory for storing instructions that, when executed by the processor cause the system to: deliver stimulation to a first peripheral nerve for a prespecified amount of time; and vary each of a first parameter and a second parameter of the delivered stimulation based on adaptive learning over a prespecified range at a prespecified rate of variation, wherein the adaptive learning employs at least one of kinematic measurements or satisfaction data.

In some embodiments, disclosed is a method of stimulating a first peripheral nerve to introduce variability to enhance therapeutic response for a user. In some embodiments, the method comprises positioning a first peripheral nerve electrode configured to be positioned to deliver stimulation to a first peripheral nerve; generating a stimulation waveform configured to be delivered with the first peripheral nerve electrode for a time period; and delivering the generated stimulation waveform to the first peripheral nerve electrode for the time period by varying one or more parameters of the stimulation waveform to avoid a constant value for the one or more parameters during the time period, wherein the variation in the one or more parameters enhances therapeutic response and/or comfort of stimulation sensations compared to maintaining the one or more parameters constant over the time period.

In some embodiments, the one or more parameters are not correlated with characteristics of the user.

In some embodiments, the varying of the one or more parameters is configured to prevent habituation to the delivered stimulation.

In some embodiments, the varying of the one or more parameters is configured to activate neuronal populations of the nerve.

In some embodiments, the varying of the one or more parameters is configured to avoid tolerance effects by the individual.

In some embodiments, the varying of the one or more parameters is configured to resemble physiological neural signaling.

In some embodiments, the processor and the memory are further configured to, when executed by the processor, cause the system to determine the value of the varied parameter based on a prespecified probabilistic distribution.

In some embodiments, the probabilistic distribution is Gaussian.

In some embodiments, the probabilistic distribution is uniform.

In some embodiments, the one or more parameters of the first stimulus comprises a first parameter and a second parameter, and wherein the first parameter and the second parameter are simultaneously or alternately varied.

In some embodiments, disclosed is a neurostimulation system configured to introduce variability to enhance therapeutic response for a user comprising: a first peripheral nerve electrode configured to be positioned to deliver stimulation to a first peripheral nerve; and a processor and a memory for storing instructions that, when executed by the processor cause the system to: generate a biphasic stimulation waveform comprising a plurality of pulses and configured to be delivered with the first peripheral nerve electrode for a time period; alternating a leading phase of the plurality of pulses during the time period; and deliver the generated biphasic stimulation waveform to the first peripheral nerve electrode for the time period.

In some embodiments, disclosed is a method of stimulating a first peripheral nerve to introduce variability to enhance therapeutic response for a user comprising: positioning a first peripheral nerve electrode configured to be positioned to deliver stimulation to a first peripheral nerve; generating a biphasic stimulation waveform comprising a plurality of pulses and configured to be delivered with the first peripheral nerve electrode for a time period; and delivering the generated biphasic stimulation waveform to the first peripheral nerve electrode for the time period by alternating a leading phase of the plurality of pulses during the time period.

In some embodiments, disclosed is a neuromodulation device for modulating one or more nerves of a user, the device comprising: means configured to generate neuromodulation; means for delivering stimulation to a first peripheral nerve; and one or more hardware processors configured to: generate a biphasic stimulation waveform comprising a plurality of pulses and configured to be delivered with the means for delivering stimulation for a time period; and deliver the generated biphasic stimulation waveform to the means for delivering stimulation for the time period by alternating a leading phase of the plurality of pulses during the time period.

In some embodiments, the leading phase alternates between a cathodic-first phase and an anodic-first phase. In some embodiments, the leading phase is maintained for a series of the plurality of pulses. In some embodiments, the series is at least two of the plurality of pulses. In some embodiments, the series comprises a burst of three of the plurality of pulses. In some embodiments, the alternating of the leading phase causes current to initially flow from the first peripheral nerve electrode during a pulse of the plurality of pulses and then initially flow to the first peripheral nerve electrode during a subsequent pulse of the plurality of pulses. In some embodiments, the biphasic stimulation waveform includes a burst frequency, and wherein a range of the burst frequency is to 3-12 Hz, and the rate of variation is 0.001-100 Hz/s. In some embodiments, the biphasic stimulation waveform includes a burst frequency, and wherein a range of the burst frequency overlaps an expected frequency range of the user. In some embodiments, the biphasic stimulation waveform includes a burst frequency, and wherein a range of the burst frequency mimics an expected frequency range of the user. In some embodiments, the biphasic stimulation waveform includes a burst frequency, and wherein a range of the burst frequency is 2-3 Hz during the time period. In some embodiments, the biphasic stimulation waveform includes a burst frequency, and wherein a range of the burst frequency is not constant during the time period. In some embodiments, the biphasic stimulation waveform includes a pulse frequency, a range of the pulse frequency is 50-150 Hz, and a rate of variation in pulse frequency is 0.001-10,000 Hz/s. In some embodiments, the biphasic stimulation waveform includes pulse frequency, and wherein a range of the pulse frequency includes two or more of 50 Hz, 100 Hz, and 150 Hz. In some embodiments, the biphasic stimulation waveform includes a pulse frequency, and wherein a range of the pulse frequency is selected to generate activity in the brain that modulates pathological cortical dynamics associated with a plurality of different users. In some embodiments, the biphasic stimulation waveform includes a pulse width, a range of the pulse width is a minimum value from one of 100, 150, 200, 250, 300, or 350 microseconds and a maximum pulse width based on the user's comfort level at a fixed stimulation amplitude, and wherein a rate of variation of the pulse width is 0.01-10,000 microseconds per second. In some embodiments, the biphasic stimulation waveform includes a stimulation amplitude, a range of the stimulation amplitude is a minimum set to the stimulation amplitude at the user's minimum sensory threshold and a maximum set to the stimulation amplitude at the user's maximum comfort level, and a rate of variation in the stimulation amplitude is 0.001-100 mA/s. In some embodiments, the biphasic stimulation waveform includes a stimulation amplitude, and wherein the stimulation amplitude is based on the user's sensory level. In some embodiments, the biphasic stimulation waveform includes a stimulation amplitude, and wherein the range is a minimum set to a stimulation amplitude at a pre-specified increment below a user's minimum sensory threshold (sub-sensory) and a maximum set to a stimulation amplitude at a user's maximum comfort level, and wherein the rate of variation is 0.001-100 mA/s.

In some embodiments, disclosed is a neurostimulation system configured to introduce variability to enhance therapeutic response for a user, the neurostimulation system comprising: a first peripheral nerve electrode configured to be positioned to deliver stimulation to a first peripheral nerve; and a processor and a memory for storing instructions that, when executed by the processor cause the system to: generate a stimulation waveform configured to be delivered with the first peripheral nerve electrode for a time period; vary one or more parameters of the stimulation waveform to avoid a constant value for the one or more parameters during the time period, wherein the one or more parameters correlate with a characteristic of the user; and deliver the generated stimulation waveform to the first peripheral nerve electrode for the time period, wherein the variation in the one or more parameters enhances therapeutic response and/or comfort of stimulation sensations compared to maintaining the one or more parameters constant over the time period.

In some embodiments, disclosed is a method of stimulating a first peripheral nerve to introduce variability to enhance therapeutic response for a user, the method comprising: positioning a first peripheral nerve electrode configured to be positioned to deliver stimulation to a first peripheral nerve; generating a stimulation waveform configured to be delivered with the first peripheral nerve electrode for a time period; and delivering the generated stimulation waveform to the first peripheral nerve electrode for the time period by varying one or more parameters of the stimulation waveform to avoid a constant value for the one or more parameters during the time period, wherein the one or more parameters correlate with a characteristic of the user, and wherein the variation in the one or more parameters enhances therapeutic response and/or comfort of stimulation sensations compared to maintaining the one or more parameters constant over the time period.

In some embodiments, disclosed is a neuromodulation device for modulating one or more nerves of a user, the device comprising: means configured to generate neuromodulation; means for delivering stimulation to a first peripheral nerve; and one or more hardware processors configured to: generate a stimulation waveform configured to be delivered with the means for delivering stimulation for a time period; and deliver the generated stimulation waveform to the means for delivering stimulation for the time period by varying one or more parameters of the stimulation waveform to avoid a constant value for the one or more parameters during the time period, wherein the one or more parameters correlate with a characteristic of the user, and wherein the variation in the one or more parameters enhances therapeutic response and/or comfort of stimulation sensations compared to maintaining the one or more parameters constant over the time period.

In some embodiments, the characteristic of the user relates to a phase of a tremor exhibited by the user. In some embodiments, the phase of the tremor is between 0 and 180 degrees. In some embodiments, the phase of the tremor is between 180 and 360 degrees. In some embodiments, the phase relates to movement of an upper extremity of the user. In some embodiments, the upper extremity is a hand, and wherein the movement is in a downward direction. In some embodiments, the upper extremity is a hand, and wherein the movement is in an upward direction. In some embodiments, the first peripheral nerve is a median nerve. In some embodiments, the first peripheral nerve is a radial or ulnar nerve. In some embodiments, the median nerve is modulated (e.g., stimulated) along with one, two or more other nerves in the same device or a separate device. For example, the median nerve and one or both of the radial and ulnar nerves are modulated in the same device. Optionally, another device to modulate (e.g., stimulate) in or around the ear or leg is also provided to provide synergy and may be, in one embodiment controlled by a common controller. In some embodiments, the one or more parameters includes burst frequency, and wherein the burst frequency relates to the phase of the tremor, and wherein the first peripheral nerve is stimulated during a first portion (e.g., 0 to 180 degrees) of the phase of the tremor and a second peripheral nerve is stimulated during a second portion (e.g., 180 to 360 degrees) of the phase of the tremor. In some embodiments, a range of burst frequency is 3-12 Hz. In some embodiments, a rate of variation in burst frequency is 0.001-100 Hz/s. In some embodiments, a range of burst frequency overlaps an expected frequency range of the characteristic of the user. In some embodiments, a range of burst frequency mimics an expected frequency range of the characteristic of the user. In some embodiments, a range of burst frequency is not constant during the time period. In some embodiments, the characteristic of the user relates to a respiratory cycle of the user, and wherein the first peripheral nerve is stimulated during a first portion (e.g., exhalation phase) of the respiratory cycle and is not stimulated during a second portion (e.g., inhalation phase) of the respiratory cycle. In some embodiments, the respiratory cycle comprises an inspiratory phase. In some embodiments, the respiratory cycle comprises a respiratory phase. In some embodiments, the inspiratory phase relates to a period of the respiratory cycle in which the user inhales. In some embodiments, the respiratory phase relates to a period of the respiratory cycle in which the user exhales. In some embodiments, the one or more parameters includes burst frequency, and wherein the burst frequency relates to the respiratory cycle of the user. In some embodiments, a range of burst frequency is 3-12 Hz. In some embodiments, a rate of variation in burst frequency is 0.001-100 Hz/s. In some embodiments, a range of burst frequency overlaps an expected frequency range of the characteristic of the user. In some embodiments, a range of burst frequency mimics an expected frequency range of the characteristic of the user. In some embodiments, a range of burst frequency is not constant during the time period.

In some embodiments, disclosed is a neurostimulation system configured to introduce variability to enhance therapeutic response for a user, the neurostimulation system comprising: a first peripheral nerve electrode configured to be positioned to deliver stimulation to a first peripheral nerve; and a processor and a memory for storing instructions that, when executed by the processor cause the system to: generate a stimulation waveform configured to be delivered with the first peripheral nerve electrode for a time period, the stimulation waveform comprising a plurality of burst; ramping up an amplitude within each burst of the plurality of bursts to avoid a constant value for the amplitude within the time period; and deliver the generated stimulation waveform to the first peripheral nerve electrode for the time period, wherein the ramping of the amplitude enhances therapeutic response and/or comfort of stimulation sensations compared to maintaining the amplitude constant over the time period.

In some embodiments, disclosed is a method of stimulating a first peripheral nerve to introduce variability to enhance therapeutic response for a user, the method comprising: positioning a first peripheral nerve electrode configured to be positioned to deliver stimulation to a first peripheral nerve; generating a stimulation waveform configured to be delivered with the first peripheral nerve electrode for a time period, the stimulation waveform comprising a plurality of burst; and delivering the plurality of bursts to the first peripheral nerve electrode for the time period by ramping up an amplitude within each burst of the plurality of bursts to avoid a constant value for the amplitude within the time period, wherein the ramped amplitude enhances therapeutic response and/or comfort of stimulation sensations compared to maintaining the amplitude constant over the time period.

In some embodiments, disclosed is a neuromodulation device for modulating one or more nerves of a user, the device comprising: means configured to generate neuromodulation; means for delivering stimulation to a first peripheral nerve; and one or more hardware processors configured to: generate a stimulation waveform configured to be delivered with the means for delivering stimulation for a time period, the stimulation waveform comprising a plurality of burst; and delivering the plurality of bursts to the means for delivering stimulation for the time period by ramping up an amplitude within each burst of the plurality of bursts to avoid a constant value for the amplitude within the time period, wherein the ramped amplitude enhances therapeutic response and/or comfort of stimulation sensations compared to maintaining the amplitude constant over the time period.

In some embodiments, a range of the amplitude includes a minimum set to a stimulation amplitude at the user's minimum sensory threshold. In some embodiments, a range of the amplitude includes a maximum set to a stimulation amplitude at the user's maximum comfort level. In some embodiments, a rate of ramping up the amplitude is 0.001-100 mA/s. In some embodiments, a range of the amplitude is a minimum set to a stimulation amplitude at a pre-specified increment below a user's minimum sensory threshold (sub-sensory). In some embodiments, the ramping up the amplitude is configured to prevent habituation to the delivered stimulation. In some embodiments, the ramping up the amplitude is configured to activate neuronal populations of the nerve. In some embodiments, the ramping up the amplitude is configured to avoid tolerance effects by the individual. In some embodiments, the ramping up the amplitude is configured to resemble physiological neural signaling. In some embodiments, the ramping up the amplitude is configured to generate a natural characteristic of neuronal activity over the time period. In some embodiments, the processor and the memory are further configured to, when executed by the processor, cause the system to determine the value of the amplitude based on a prespecified probabilistic distribution. In some embodiments, the probabilistic distribution is Gaussian. In some embodiments, the probabilistic distribution is uniform.

In some embodiments, disclosed is a neurostimulation system configured to introduce variability to enhance therapeutic response for a user, the neurostimulation system comprising: a first peripheral nerve electrode configured to be positioned to deliver stimulation to a first peripheral nerve; and a processor and a memory for storing instructions that, when executed by the processor cause the system to: generate a stimulation waveform configured to be delivered with the first peripheral nerve electrode during a time period, the time period comprising an on cycle portion and an off cycle portion; and deliver the generated stimulation waveform to the first peripheral nerve electrode only during the on cycle portion of the time period, wherein the off cycle portion enhances therapeutic response and/or comfort of stimulation sensations compared to maintaining a continuous stimulation over the time period.

In some embodiments, disclosed is a method of stimulating a first peripheral nerve to introduce variability to enhance therapeutic response for a user, the method comprising: positioning a first peripheral nerve electrode configured to be positioned to deliver stimulation to a first peripheral nerve; generating a stimulation waveform configured to be delivered with the first peripheral nerve electrode for a time period, the time period comprising an on cycle portion and an off cycle portion; and delivering the generated stimulation waveform to the first peripheral nerve electrode only during the on cycle portion of the time period, wherein the off cycle portion enhances therapeutic response and/or comfort of stimulation sensations compared to maintaining a continuous stimulation over the time period.

In some embodiments, disclosed is a neuromodulation device for modulating one or more nerves of a user, the device comprising: means configured to generate neuromodulation; means for delivering stimulation to a first peripheral nerve; and one or more hardware processors configured to: generate a stimulation waveform configured to be delivered with the means for delivering stimulation for a time period, the time period comprising an on cycle portion and an off cycle portion; and deliver the generated stimulation waveform to the means for delivering stimulation only during the on cycle portion of the time period, wherein the off cycle portion enhances therapeutic response and/or comfort of stimulation sensations compared to maintaining a continuous stimulation over the time period.

In some embodiments, the on-cycle portion comprises three cycles. In some embodiments, the off-cycle portion comprises two cycles. In some embodiments, each on cycle portion and off cycle portion each comprise one or more cycles. In some embodiments, a number of cycles for the on-cycle portion are greater than a number of cycles for the off-cycle portion. In some embodiments, the stimulation waveform comprises a plurality of bursts, and wherein one burst of the plurality of bursts is delivered to the first peripheral nerve electrode during one cycle of the on-cycle portion. In some embodiments, the on-cycle portion and the off-cycle portion are configured to prevent habituation to the delivered stimulation. In some embodiments, the on-cycle portion and the off-cycle portion are configured to activate neuronal populations of the nerve. In some embodiments, the on-cycle portion and the off-cycle portion are configured to avoid tolerance effects by the individual. In some embodiments, the on-cycle portion and the off-cycle portion are configured to resemble physiological neural signaling. In some embodiments, the on-cycle portion and the off-cycle portion are configured to avoid exact alignment with a pathological characteristic over the time period. In some embodiments, the on-cycle portion and the off-cycle portion are configured to generate a natural characteristic of neuronal activity over the time period. In some embodiments, the on-cycle portion and the off-cycle portion are configured to maximize shifting of pathological neural oscillations away from their peak resonance frequency and/or enhancing spike-timing-dependent plasticity (STDP). In some embodiments, the processor and the memory are further configured to, when executed by the processor, cause the system to determine the on-cycle portion and the off-cycle portion of the time period based on a prespecified probabilistic distribution. In some embodiments, the probabilistic distribution is Gaussian. In some embodiments, the probabilistic distribution is uniform.

In some embodiments, a neuromodulation device can comprise any one or more of the embodiments described in the disclosure.

In some embodiments, a method for performing neuromodulation on one or more nerves, comprising any one or more of the embodiments described in the disclosure.

In some embodiments, a system can comprise, not comprise, consist essentially of, or consist of any number of features as disclosed herein.

In some embodiments, a method can comprise, not comprise, consist essentially of, or consist of any number of features as disclosed herein.

Any or all of the devices or methods described herein can be used for the treatment of disorders and/or associated symptoms such as depression (including but not limited to post-partum depression, depression affiliated with neurological diseases, major depression, seasonal affective disorder, depressive disorders, etc.), inflammation (e.g., neuroinflammation), Lyme disease, stroke, neurological diseases (such as Parkinson's and Alzheimer's), and gastrointestinal issues (including those in Parkinson's disease). In some embodiments, conditions such as stroke, cardiac events, inflammation, etc. are treated.

In several embodiments, one or more of bradykinesia, dyskinesia, gait dysfunction, dystonia and/or rigidity are treated with the devices and methods described herein (e.g., in connection with Parkinson's disease or in connection with other disorders). Rehabilitation of movement is treated in some embodiments (for example to restore or improve movement and motion) in subjects who have suffered from an acute or chronic event, including, for example, cardiac events (such as atrial fibrillation, hypertension, and stroke), inflammation, neuroinflammation, etc. Epilepsy is treated in one embodiment. Treatment of movement disorders herein also includes, for example, treatment of involuntary and/or repetitive movements, such as tics, twitches, etc. (including, but not limited to, Tourette Syndrome, tic disorders for example). Rhythmic and/or non-rhythmic involuntary movements may be controlled in several embodiments. Involuntary vocal tics and other vocalizations may also be treated. Rehabilitation of movement can include, for example, rehabilitation of limb movement. In some embodiments, provided herein are treatments of restless leg syndrome, periodic limb movement disorder, repetitive movements of the limbs and abnormal sensation. Devices described herein can be placed, for example, on the wrist or leg (or both) to treat leg disorders. One, two, three or more nerves may be treated including for example, peroneal, saphenous, tibial, femoral, and sural. In some embodiments, two, three or more nerves are treated. A band or other device may be placed on a wrist and the leg, only on the wrist or leg, or on two or more locations on one or both limbs. A single device, two or more devices that are coupled physically and/or in communication with each other may be used. Stimulation may be automated, user-controllable, or both.

In some embodiments, disorders and symptoms caused or exacerbated by microbial infections (e.g., bacteria, viruses, fungi, and parasites) are treated. Symptoms include but are not limited to sympathetic/parasympathetic imbalance, autonomic dysfunction, inflammation (e.g., neuroinflammation), inflammation, motor and balance dysfunction, pain and other neurological symptoms. Disorders include but are not limited to tetanus, meningitis, Lyme disease, urinary tract infection, mononucleosis, chronic fatigue syndrome, autoimmune disorders, etc. In some embodiments, autoimmune disorders and/or pain unrelated to microbial infection are treated, including for example, inflammation (e.g., neuroinflammation), headache, back pain, joint pain and stiffness, muscle pain and tension, etc.

Any or all of the devices described herein can be used for the treatment of inflammatory bowel disease (such as Crohn's disease), rheumatoid arthritis, multiple sclerosis, psoriatic arthritis, osteoarthritis, psoriasis and other inflammatory diseases. Any or all of the devices described herein can be used for the treatment of inflammatory skin conditions. Any or all of the devices described herein can be used for the treatment chronic inflammatory symptoms and flare ups. Systems and methods to reduce habituation and/or tolerance to stimulation in the disorders and symptoms identified herein are provided in several embodiments by, for example, introducing variability in stimulation parameter(s) described herein.

Any or all of the devices described herein can be used for the treatment of cardiac conditions (such as atrial fibrillation, hypertension, and stroke). Epilepsy is treated in one embodiment. Any or all of the devices described herein can be used for the treatment of immune dysfunction. Any or all of the devices described herein can be used to stimulate the autonomic nervous system. Any or all of the devices described herein can be used to balance the sympathetic/parasympathetic nervous systems.

For purposes of summarizing the disclosure, certain aspects, advantages, and novel features are discussed herein. It is to be understood that not necessarily all such aspects, advantages, or features will be embodied in any particular embodiment of the disclosure, and an artisan would recognize from the disclosure herein a myriad of combinations of such aspects, advantages, or features.

### BRIEF DESCRIPTION OF THE DRAWINGS

Non-limiting features of some embodiments of the invention are set forth with particularity in the claims that follow. The following drawings are for illustrative purposes only and show non-limiting embodiments. Features from different figures may be combined in several embodiments.

FIG. 1A illustrates a block diagram of an example neuromodulation (e.g., neurostimulation) device.

FIG. 1B illustrates a block diagram of an embodiment of a user interface device that can be implemented with the hardware components described with respect to FIG. 1A. Communications between the neurostimulation device of FIG. 1A and the user interface device over a communication link are illustrated in FIG. 1B.

FIG. 1C schematically illustrates an embodiment of a neuromodulation device and base station.

FIG. 2 illustrates a block diagram of an embodiment of a controller that can be implemented with the hardware components described with respect to FIGS. 1A to 1C.

FIGS. 3A-3C illustrate three stimulation channels where the common electrodes of the band are positioned near the radial nerve and the medial nerve, respectively. In the embodiments of FIGS. 3A-3C, the primary target nerve is the radial nerve.

FIGS. 4A-4C illustrate three stimulation channels where the common electrodes of the band are positioned near the radial nerve and the medial nerve, respectively. In the embodiments of FIGS. 4A-4C, the primary target nerve is the median nerve.

FIGS. 5A-5C include nerve stimulation plots of percentage activation v. current for the embodiments illustrated in FIGS. 3A-3C and 4A-4C. The plots show different patterns of activation for different stimulation channels.

FIGS. 6A and 6B represent stimulation patterns including a pattern which alternates between two different channels and a pattern which cycles through six different channels during treatment.

FIG. 7 is a graph of stimulation amplitude v. time showing a temporary, high stimulation amplitude being applied to promote the therapeutic response of the user.

FIG. 8 is a graph of stimulation amplitude v. time showing a periodic, high stimulation amplitude being applied to promote the therapeutic response of the user.

FIG. 9 is a graph of kinematic tremor measurements taken during treatment and used to determine whether a change in stimulation amplitude will promote the therapeutic response of the user.

FIG. 10 illustrates a block diagram of an embodiment of a device and system that provides peripheral nerve stimulation and senses a biological or kinematic measure and/or receives user satisfaction data that is used to customize or modify the delivery of an electrical stimulus.

FIG. 11 illustrates a block diagram of an embodiment of a controller that can be implemented with the hardware components described with respect to FIGS. 1A, 1B, 1C, and 10.

FIGS. 12A-C2 illustrate examples of how stimulation parameters (e.g., burst frequency, pulse frequency, and pulse phase) are varied between two or more prespecified values as stimulation is alternated across two nerves (e.g., median and radial or ulnar nerves).

FIGS. 12D1-E illustrate examples of how stimulation parameters (e.g., pulse frequency) are varied between two or more values based on physiological parameters (e.g., tremor frequency and respiration rate) as stimulation is alternated across two nerves (e.g., median and radial or ulnar nerves).

FIGS. 13A-C illustrate examples of how stimulation parameters (e.g., amplitude and pulse width) are varied between two or more prespecified values as stimulation is alternated across two nerves (e.g., median and radial or ulnar nerves).

FIGS. 14A-C illustrate multiple examples of stimulation patterns with prespecified on/off periods as stimulation is alternated across two nerves (e.g., median and radial or ulnar nerves).

FIG. 15A illustrates an example of a ramping variation of the burst frequency parameter. The burst frequency linearly ramps from 3 Hz to 12 Hz in time period of 2 seconds, which results in a rate of change of 4.5 Hz/s.

FIG. 15B illustrates an example of a ramping variation of the burst frequency parameter. The burst frequency linearly ramps from 3 Hz to 3.4 Hz in time period of 5 seconds, which results in a rate of change of 0.08 Hz/s.

FIG. 16 illustrate an example of how multiple stimulation parameters (e.g., parameters A and B) are simultaneously varied between two or more prespecified values as stimulation is applied to a nerve (e.g., median or radial or ulnar nerve).

FIG. 17 illustrate an example of how multiple stimulation parameters (e.g., parameters A and B) are varied by alternately changing each parameter between two or more prespecified values as stimulation is applied to a nerve (e.g., median or radial or ulnar nerve).

FIG. 18 illustrate an example of how multiple stimulation parameters (e.g., parameters A and B) are varied by applying different timescales to each parameter as stimulation is applied to a nerve (e.g., median or radial or ulnar nerve).

FIG. 19 illustrates a flow chart of an embodiment of a process for varying one or more parameters of a stimulus over a prespecified range of parameters at a prespecified rate of variation.

FIG. 20 illustrates a flow chart of an embodiment of a process for simultaneously varying multiple stimulation parameters (e.g., parameters A and B) between two or more prespecified values as stimulation is applied to a nerve (e.g., median or radial or ulnar nerve).

FIG. 21 illustrates a flow chart of an embodiment of a process for alternately varying multiple stimulation parameters (e.g., parameters A and B) between two or more prespecified values as stimulation is applied to a nerve (e.g., median or radial or ulnar nerve).

FIG. 22 illustrates a flow chart of an embodiment of a process for varying multiple stimulation parameters (e.g., parameters A and B) between two or more prespecified values by applying different timescales to each parameter as stimulation is applied to a nerve (e.g., median or radial or ulnar nerve).

FIG. 23 illustrates an architecture for determining a method that varies multiple stimulation parameters based on adaptive learning.

### DETAILED DESCRIPTION

Disclosed herein are devices configured for providing neuromodulation (e.g., neurostimulation). The neuromodulation (e.g., neurostimulation) devices provided herein may be configured to stimulate peripheral nerves of a user. The neuromodulation (e.g., neurostimulation) devices may be configured to transmit one or more neuromodulation (e.g., neurostimulation) signals across the skin of the user. In many embodiments, the devices are wearable devices configured to be worn by a user. The user may be a human, another mammal, or other animal user. The neuromodulation (e.g., neurostimulation) system could also include signal processing systems and methods for enhancing diagnostic and therapeutic protocols relating to the same. In some embodiments, the neuromodulation (e.g., neurostimulation) device is configured to be wearable on an upper extremity of a user (e.g., a wrist, forearm, arm, and/or finger(s) of a user). In some embodiments, the device is configured to be wearable on a lower extremity (e.g., ankle, calf, knee, thigh, foot, and/or toes) of a user. In some embodiments, the device is configured to be wearable on the head or neck (e.g., forehead, ear, neck, nose, and/or tongue). In several embodiments, dampening or blocking of nerve impulses and/or neurotransmitters are provided. In some embodiments, nerve impulses and/or neurotransmitters are enhanced. In some embodiments, the device is configured to be wearable on or proximate an ear of a user, including but not limited to auricular neuromodulation (e.g., neurostimulation) of the auricular branch of the vagus nerve, for example. One or more of the auriculotemporal, great auricular, vagus, trigeminal or cranial nerves may be treated in some embodiments. The device could be unilateral or bilateral, including a single device or multiple devices connected with wires or wirelessly. Transcutaneous neuromodulation is provided in several embodiments, although subcutaneous and percutaneous components may also be used. In some embodiments, the device includes three to six or more electrodes (e.g., 3, 4, 5, 6), and is partially implantable or is entirely transcutaneous.

In some embodiments, features disclosed for example in U.S. Pat. No. 9,452,287 to Rosenbluth et al., U.S. Pub. No. 2019/0001129 to Rosenbluth et al., U.S. Pat. No. 9,802,041 to Wong et al., and U.S. Pub. No. 2018/0169400 to Wong et al., each of which are hereby incorporated by reference in their entireties, can be combined with systems and methods as disclosed herein.

In some embodiments, modulation of the blood vessel (either dilation or constriction) is provided using the devices and methods described herein (e.g., through nerve stimulation). Such therapy may, in turn, reduce inflammation (including but not limited to inflammation post microbial infection). The devices and methods described herein increase, decrease or otherwise balance vasodilation and vasoconstriction through neuromodulation in some embodiments. For example, reduction of vasodilation is provided in several embodiments to treat or prevent migraine or other conditions that are aggravated by vasodilation. In other embodiments, vasoconstriction is reduced in, for example, conditions in which dilation is beneficial (such as with high blood pressure and pain). In one embodiment, reduction in inflammation treats tinnitus. In some embodiments, modulation of the blood vessel (either dilation or constriction) is used to treat tinnitus. Tinnitus may be treated according to several embodiments through modulation (e.g., stimulation) of the vagus nerve alone or in conjunction with one, two or more other nerves (including for example the trigeminal nerve, great auricular nerve, nerves of the auricular branch, auricular branch of the vagus nerve, facial nerve, the auriculotemporal nerve, etc.). In one embodiment, nerves other than the vagus nerve are modulated to treat tinnitus. Cranial/auditory nerves may be modulated to treat tinnitus and/or auricular inflammation in some embodiments. Auricular devices may be used in conjunction with devices placed on limbs to in some embodiments (e.g., an ear device along with a wrist device).

Any of the neuromodulation devices discussed herein can be utilized to modulate (e.g., stimulate) median, radial, ulnar, sural, femoral, peroneal, saphenous, tibial and/or other nerves or meridians accessible on the limbs of a subject alone or in combination with a one or more other nerves (e.g., vagal nerve) in the subject, for example, via a separate neuromodulation device. In some embodiments, provided herein are treatments of restless leg syndrome, periodic limb movement disorder, repetitive movements of the limbs and abnormal sensation. Devices described herein can be placed, for example, on the wrist or leg (or both) to treat limb disorders. In some embodiments, vagus nerve stimulation is used to treat restless leg syndrome, periodic limb movement disorder, repetitive movements of the limbs and/or abnormal limb sensation. The vagus nerve may be stimulated alone or in addition to one or more of the sural, femoral, peroneal, saphenous, and tibial nerves. Alternatively, one or more of the sural, femoral, peroneal, saphenous, and tibial nerves are stimulated without stimulating the vagus nerve.

In some embodiments, transcutaneous nerve neuromodulation at the arm and/or wrist (e.g., median and/or radial or ulnar nerve stimulation) can advantageously inhibit sympathoexcitatory related increases in blood pressure and premotor sympathetic neural firing in the rostral ventrolateral medulla (rVLM). Neuromodulation of the median and/or radial or ulnar nerves, for example, can provide more convergent input into cardiovascular premotor sympathetic neurons in the rVLM. In some embodiments, the median nerve is modulated (e.g., stimulated) along with one, two or more other nerves in the same device or a separate device. For example, the median nerve and one or both of the radial and ulnar nerves are modulated in the same device. Optionally, another device to modulate (e.g., stimulate) in or around the ear or leg is also provided to provide synergy and may be, in one embodiment controlled by a common controller. In some embodiments, the device(s) can also be configured to deliver one, two or more of the following: magnetic, vibrational, mechanical, thermal, ultrasonic, or other forms of modulation (e.g., stimulation) instead of, or in addition to electrical stimulation. Different types of modulation can be provided in the same device or in different devices. For example, a device on the wrist can provide electrical and vibrational stimulation or, for example, a device on the wrist can provide electrical stimulation while a device on the leg provides vibrational stimulation.

Also, in some embodiments, vagal nerve stimulation can modulate the trigeminal nuclei to inhibit inflammation. Thus, in several embodiments the vagal nerve is stimulated to reduce inflammation via a trigeminal pathway. In other embodiments, the trigeminal nerve is stimulated directly instead of or in addition to the vagus nerve. In some embodiments, transcutaneous nerve stimulation projects to the nucleus tractus solitarii (NTS) and spinal trigeminal nucleus (Sp5) regions to modulate trigeminal sensory complex excitability and connectivity with higher brain structures. Trigeminal sensory nuclei can be involved in neurogenic inflammation during migraine (e.g., characterized by vasodilation). In some embodiments, stimulation of the nerve modulates the trigeminal sensory pathway to ameliorate migraine pathophysiology and reduce headache frequency and severity. For example, increased activation of raphe nuclei and locus coeruleus may inhibit nociceptive processing in the sensory trigeminal nucleus. Human skin is well innervated with autonomic nerves and neuromodulation (e.g., stimulation) of nerve or meridian points as disclosed herein can potentially help in treatment of migraine or other headache conditions. For example, transcutaneous nerve stimulation of afferent nerves in the periphery or distal limbs, including but not limited to median nerve, are connected by neural circuits to the arcuate nucleus of the hypothalamus. In some embodiments, the devices and methods described herein increase, decrease or otherwise balance vasodilation and vasoconstriction through neuromodulation (such as the vagus nerve, trigeminal nerve and/or other nerves surrounding the ear). For example, reduction of vasodilation is provided in several embodiments to treat or prevent migraine or other conditions that are exacerbated by vasodilation. In other embodiments, vasoconstriction is reduced in, for example, conditions in which dilation is beneficial (such as with high blood pressure and pain). In some embodiments, modulation of the blood vessel (either dilation or constriction) is used to treat tinnitus. In one embodiment, the devices and methods described herein reduce inflammation (including but not limited to inflammation post microbial infection), and the reduction in inflammation treats tinnitus.

Single or multiple bands that partially or fully encircle a limb (such as a wrist, ankle, arm, leg) are provided in some embodiments. Ear devices are also provided in some embodiments that can be used with or without a limb band. In one embodiment, an ear device and a wrist band are provided for synergistic treatment. An auricular (e.g., ear) device can include an earpiece or bud for one or more portions of the ear such as an ear canal or external ear. One to six or more electrodes may be placed on the earpiece or bud, or on a device connected to the earpiece/bud. Right, left or two earpieces are provided in some embodiments.

Systems with compact, ergonomic form factors are needed to enhance efficacy, compliance, and/or comfort when using non-invasive or wearable neuromodulation devices. In several embodiments, neuromodulation systems and methods are provided that enhance or inhibit nerve impulses and/or neurotransmission, and/or modulate excitability of nerves, neurons, neural circuitry, and/or other neuroanatomy that affects activation of nerves and/or neurons. For example, neuromodulation (e.g., neurostimulation) can include one or more of the following effects on neural tissue: depolarizing the neurons such that the neurons fire action potentials; hyperpolarizing the neurons to inhibit action potentials; depleting neuron ion stores to inhibit firing action potentials; altering with proprioceptive input; influencing muscle contractions; affecting changes in neurotransmitter release or uptake; and/or inhibiting firing.

Although several neurostimulation devices are described herein, in some embodiments nerves are modulated non-invasively to achieve neuro-inhibition. Neuro-inhibition can occur in a variety of ways, including but not limited to hyperpolarizing the neurons to inhibit action potentials and/or depleting neuron ion stores to inhibit firing action potentials. This can occur in some embodiments via, for example, anodal or cathodal stimulation, low frequency stimulation (e.g., less than about 5 Hz, 100 Hz, 150 Hz, 200 Hz, in some cases), or continuous or intermediate burst stimulation (e.g., theta burst stimulation). This can occur in some embodiments via, for example, anodal or cathodal stimulation, high frequency stimulation (e.g., greater than about 1 kHz in some cases), or continuous or intermediate burst stimulation (e.g., theta burst stimulation). In some embodiments, the wearable devices have at least one implantable portion, which may be temporary or more long term. In many embodiments, the devices are entirely wearable and non-implantable.

Stimulation of peripheral nerves can provide therapeutic benefit across a variety of diseases, including but not limited to disorders and/or associated symptoms such as movement disorders (including but not limited to essential tremor, Parkinson's tremor, orthostatic tremor, and multiple sclerosis), urological disorders, gastrointestinal disorders, cardiac diseases, inflammatory diseases, mood disorders (including but not limited to depression, bipolar disorder, dysthymia, and anxiety disorder), pain syndromes (including but not limited to migraines and other headaches, trigeminal neuralgia, fibromyalgia, complex regional pain syndrome), Lyme disease, stroke, among others. Inflammatory bowel disease (such as Crohn's disease, colitis, and functional dyspepsia), rheumatoid arthritis, multiple sclerosis, psoriatic arthritis, psoriasis, chronic fatigue syndrome, and other inflammatory diseases are treated in several embodiments. Cardiac conditions (such as atrial fibrillation, hypertension, and stroke) are treated in one embodiment. Epilepsy is treated in one embodiment. Inflammatory skin conditions and immune dysfunction are also treated in some embodiments. In some embodiments, provided herein are treatments of restless leg syndrome, periodic limb movement disorder, repetitive movements of the limbs and abnormal sensation. Devices described herein can be placed, for example, on the wrist or leg (or both) to treat limb disorders. In some embodiments, vagus nerve stimulation is used to treat restless leg syndrome, periodic limb movement disorder, repetitive movements of the limbs and/or abnormal limb sensation. With respect to the leg, a device may be placed, for example, on the thigh, calf, ankle or other location suitable to treat the target nerve(s).

In several embodiments, one or more of bradykinesia, dyskinesia, gait dysfunction, dystonia and/or rigidity are treated. These may be treated in connection with Parkinson's disease or in connection with other disorders. Rehabilitation of movement is treated in some embodiments (for example to restore or improve movement and motion) in subjects who have suffered from an acute or chronic event including, for example, cardiac events (such as atrial fibrillation, hypertension, and stroke), inflammation, neuroinflammation, etc. Epilepsy is treated in one embodiment. Rehabilitation of movement can include, for example, rehabilitation of limb movement. In some embodiments, provided herein are treatments of restless leg syndrome, periodic limb movement disorder, repetitive movements of the limbs and abnormal sensation. Devices described herein can be placed, for example, on the wrist or leg (or both) to treat leg disorders. One or more nerves may be treated including for example, peroneal, saphenous, tibial, femoral, and sural. In some embodiments, two, three or more nerves are treated. A band or other device may be placed on a wrist and the leg, only on the wrist or leg, or on two or more locations on one or both limbs. A single device, two or more devices that are coupled physically and/or in communication with each other may be used. Stimulation may be automated, user-controllable, or both.

In some embodiments, disorders and symptoms caused or exacerbated by microbial infections (e.g., bacteria, viruses, fungi, and parasites) are treated. Symptoms include but are not limited to sympathetic/parasympathetic imbalance, autonomic dysfunction, inflammation, motor and balance dysfunction, pain and other neurological symptoms. Disorders include but are not limited to tetanus, meningitis, Lyme disease, urinary tract infection, mononucleosis, chronic fatigue syndrome, autoimmune disorders, etc. In some embodiments, autoimmune disorders and/or pain unrelated to microbial infection is treated, including for example, inflammation (e.g., neuroinflammation), headache, back pain, joint pain and stiffness, muscle pain and tension, etc.

In some embodiments, wearable systems and methods as disclosed herein can advantageously be used to identify whether a treatment is effective in significantly reducing or preventing a medical condition, including but not limited to tremor severity. Although tremor is treated in several embodiments, the devices described herein are used to treat conditions other than tremor. Wearable sensors can advantageously monitor, characterize, and aid in the clinical management of hand tremor as well as other medical conditions including those disclosed elsewhere herein. In some embodiments, clinical ratings of medical conditions, e.g., tremor severity can correlate with simultaneous measurements of wrist motion using inertial measurement units (IMUs). For example, tremor features extracted from IMUs at the wrist can provide characteristic information about tremor phenotypes that may be leveraged to improve diagnosis, prognosis, and/or therapeutic outcomes. Kinematic measures can correlate with tremor severity, and machine learning algorithms incorporated in neuromodulation systems and methods as disclosed for example herein can predict tremor severity.

In other non-tremor embodiments, physiological data including heart rate, blood glucose, blood pressure, respiration rate, body temperature, blood volume, sound pressure, photoplethysmography, electroencephalogram, electrocardiogram, blood oxygen saturation, and/or skin conductance as well as patient data from third party devices can be collected and/or aggregated to improve diagnosis, prognosis, and/or therapeutic outcomes for migraine, depression, and/or Lyme disease. For example, physiological data including respiration rate and heart rate along with data related to sleep patterns and activity level can be collected and/or aggregated to improve diagnosis, prognosis, and/or therapeutic outcomes for depression.

In several embodiments, neuromodulation, such as neurostimulation, as used herein is used to replace pharmaceutical agents, and thus reduce undesired drug side effects. In other embodiments, neuromodulation, such as neurostimulation, is used together with (e.g., synergistically with) pharmaceutical agents to, for example, reduce the dose or duration of drug therapy, thereby reducing undesired side effects. Undesired drug side effects include for example, addiction, tolerance, dependence, GI issues, nausea, confusion, dyskinesia, altered appetite, etc.

In some embodiments, disclosed herein is a neuromodulation system to modulate one or more peripheral nerves of an arm, hand, wrist, leg, ankle, foot, head, face, neck or ear. In one embodiment, neuromodulation comprises neuromodulation of a first peripheral nerve, a processor and a memory for storing instructions that, when executed by the processor cause the device to neuromodulate a first peripheral nerve for a prespecified amount of time and vary one or more parameters over a prespecified range of parameters at a prespecified rate of variation. Parameters, include for example, burst frequency, pulse frequency, pulse width, intensity, and/or on/off cycling. Non-implantable stimulation via electrodes is provided in several embodiments. Stimulation may also be accomplished via an implantable system or a combination of an implantable element and non-implantable system. Denervation may also be accomplished in some embodiments.

In some embodiments, the one or more parameters of the first stimulus comprises a first parameter and a second parameter, and wherein the first parameter and the second parameter are varied on different timescales. In some embodiments, the one or more parameters of the first stimulus comprises a first parameter and a second parameter, wherein the first parameter and the second parameter are varied based on adaptive learning, and wherein the adaptive learning employs at least one of kinematic measurements or satisfaction data. In some embodiments, disclosed herein is a method of stimulating one or more peripheral nerves of an arm, hand, wrist, leg, ankle, foot, head, face, neck or ear with a neurostimulation device, comprising: positioning a first peripheral nerve electrode to deliver stimulation to a first peripheral nerve; delivering stimulation to a first peripheral nerve for a prespecified amount of time; and/or varying one or more parameters of the first stimulus over a prespecified range of parameters at a prespecified rate of variation, where the parameters could include burst frequency, pulse frequency, pulse width, intensity, and/or on/off cycling. In some embodiments, the varied parameter is restricted to (e.g., consists essentially of or comprises) burst frequency and the rate of variation is restricted to (e.g., consists essentially of or comprises) 0.001-100 Hz/s and the range is set by: measuring motion of the patient's extremity using the one or more biomechanical sensors to generate motion data; determining tremor frequency from the motion data; and setting the range across a 0.1, 0.2, 0.25, 0.3, 0.4, 0.5, 1, 1.5, 2, 2.5, 3, 3.5, 4, 4.5, 5, 5.5, or 6 Hz or more or less window centered on the measured tremor frequency. In some embodiments, the varied parameter is restricted to (e.g., consists essentially of or comprises) pulse width and the rate of variation is restricted to (e.g., consists essentially of or comprises) 0.01-10,000 microseconds per second, and the range is set by setting pulse width to 300 microseconds, increasing and setting stimulation amplitude to an individual's minimum sensory threshold; increasing pulse width to an individual's maximum level of comfort, recording the pulse width at maximum level of comfort, and setting the minimum range value to 300 microseconds, and the maximum range value to the individual's pulse width at maximum level of comfort. In some embodiments, the varied parameter is restricted to (e.g., consists essentially of or comprises) stimulation amplitude and the rate of variation and the rate of variation is restricted to (e.g., consists essentially of or comprises) 0.001-100 mA/s, and the range is set by: increasing the stimulation amplitude to an individual's minimum sensory threshold, setting the minimum range value to this minimum sensory threshold, increasing the stimulation amplitude to an individual's maximum comfort level, and setting the maximum range value to this maximum comfort level. In some embodiments, the varied parameter is restricted to (e.g., consists essentially of or comprises) stimulation amplitude and the rate of variation and the rate of variation is restricted to (e.g., consists essentially of or comprises) 0.001-100 mA/s, and the range is set by: increasing the stimulation amplitude to an individual's minimum sensory threshold, setting the minimum range value to a value that is 0.1, 0.2, 0.25, 0.3, 0.4, 0.5, 0.6, 0.7, 0.75, 0.8, 0.9, or 1 mA below this minimum sensory threshold, increasing the stimulation amplitude to an individual's maximum comfort level, and setting the maximum range value to this maximum comfort level. In some embodiments, the one or more parameters of the first stimulus comprises a first parameter and a second parameter, and wherein the first parameter and the second parameter are simultaneously varied. In some embodiments, the one or more parameters of the first stimulus comprises a first parameter and a second parameter, and wherein the first parameter and the second parameter are alternately varied. In some embodiments, the one or more parameters of the first stimulus comprises a first parameter and a second parameter, and wherein the first parameter and the second parameter are varied on different timescales. In some embodiments, the one or more parameters of the first stimulus comprises a first parameter and a second parameter, wherein the first parameter and the second parameter are varied based on adaptive learning, and wherein the adaptive learning employs at least one of kinematic measurements or satisfaction data.

### Neuromodulation Devices

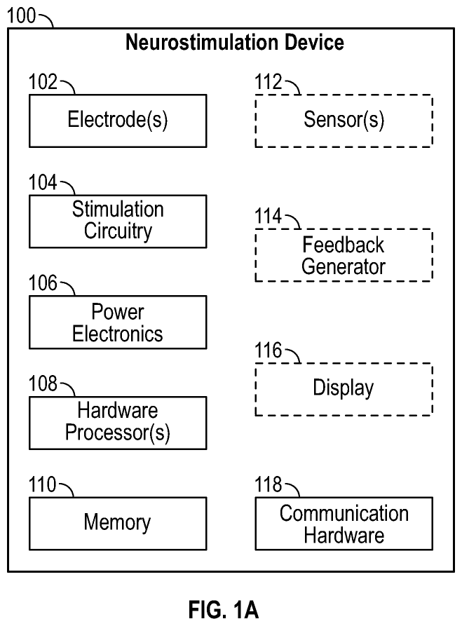

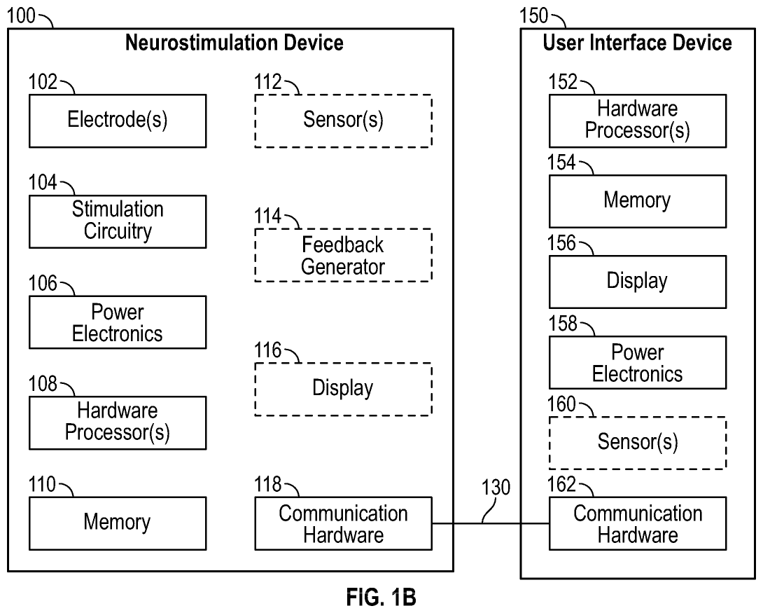

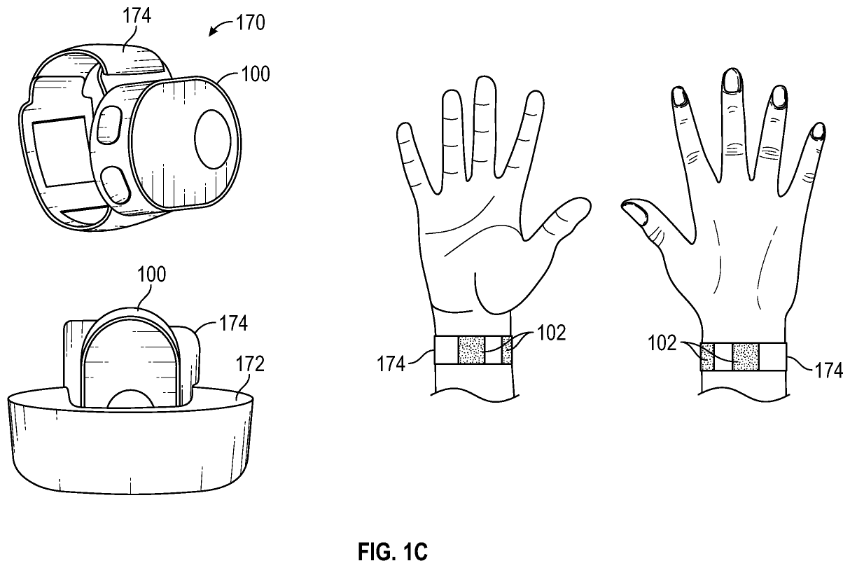

FIG. 1A illustrates a block diagram of an example neuromodulation (e.g., neurostimulation) device 100. The device 100 includes multiple hardware components which are capable of or programmed to provide therapy across the skin of the user. As illustrated in FIG. 1A, some of these hardware components may be optional as indicated by dashed blocks. In some instances, the device 100 may only include the hardware components that are required for stimulation therapy. The hardware components are described in more detail below.

The neurostimulation device 100 can include two or more effectors, e.g., electrodes 102 for providing neurostimulation signals. In some embodiments, the device 100 includes three to six or more electrodes 102 (e.g., 3, 4, 5, 6), and is partially implantable or is entirely transcutaneous. In some embodiments, 2-12 electrodes are provided (e.g., 2, 3, 4, 5, 6, 7, 8, 9, 10, 11, 12, or more). In some embodiments 3-12 or more electrodes 102 are used (e.g., 3, 6, 9 or 12). In one embodiment, none of the electrodes 102 are in contact with areas that cause discomfort.

The electrodes 102 could be percutaneous or microneedle electrodes in other embodiments, or only transcutaneous (e.g., not percutaneous, microneedles, or implanted electrodes in some embodiments). In many embodiments, the transcutaneous device 100 is a wearable band 174 or earpiece. The band 174 may partially or fully surround a wrist, finger, arm, leg, ankle or head. Patches may be used, but in many embodiments a patch is not used. Several embodiments provide a wrist worn or ear worn device, or both.

In some embodiments, the electrodes 102 have a generally rectangular shape and includes six electrodes 102. In other embodiments, the electrodes 102 have a round shape or any other shape. Changing the electrode shape can also control the excitation in an area and make the stimulation more comfortable. Square or partially rounded shapes may also be provided. Three to twelve electrodes (e.g., 3, 9, 12 etc.) may be provided in some embodiments. In one embodiment, mechanical (e.g., vibrational) stimulation may be provided before, after or during electrical stimulation for diagnostic and/or therapeutic purposes. Such stimulation may be provided via one or more mechanical/vibratory elements or bands configured to vibrate at a steady or varied frequencies (e.g., of between about 5-50 Hz, 4-60 Hz, 50-100 Hz, 50-300 Hz, 100-450 Hz and overlapping ranges therein). Likewise, the electrical stimulation parameters disclosed herein can be varied or steady within a given time frame (seconds, minutes, hours, etc.). Single or multiple frequencies can be used (e.g., two, three or more electrical stimulations and/or mechanical/vibrational stimulations) at the same, overlapping or different nerves. In one embodiment, varying frequency or other parameters reduces tolerance or habituation and/or increase patient comfort/compliance.

In some instances, the device 100 is configured for transcutaneous use only and does not include any percutaneous or implantable components. In some embodiments, the electrodes 102 can be dry electrodes. In some embodiments, water or gel can be applied to the dry electrode 102 or skin to improve conductance. In some embodiments, the electrodes 102 do not include any hydrogel material, adhesive, or the like.

The device 100 can further include stimulation circuitry 104 for generating signals that are applied through the electrode(s) 102. In certain embodiments, the signals can vary in, for example, frequency, phase, timing, amplitude, on/off cycling, or offsets. The device 100 can also include power electronics 106 for providing power to the hardware components. For example, the power electronics 106 can include a battery.

In some embodiments, the neurostimulation device 100 employs three or more electrodes 102 to apply a stimulation signal to the patient. For example, in some embodiments, at least one electrode is redundant to another electrode (e.g., 2 or more redundant common electrodes and/or 2 or more redundant stimulation electrodes). In this way, even if the electrical contact between one of the two electrodes and the patient's skin is poor increasing resistance, the electrical contact between the redundant electrode and the patient's skin can complete the electrical circuit with a normal or expected level of resistance.

In some embodiments, the 2 or more common electrodes and/or 2 or more stimulation electrodes are circumferentially spaced about the band so that even if the band rotates slightly on the wrist causing an electrode to lose contact with the patient's skin, the redundant electrode will still be in contact with the patient's skin to compete the circuit with a normal or expected level of resistance. In this way, the desired stimulation signal (e.g., frequency, phase, timing, amplitude, and/or offsets) is applied to the patient even when the band rotates on the patient's wrist. The band is less sensitive to electrical contact variations between the electrodes and the patient's skin caused by variations in the angular orientation of the band on the wrist.

The signals can vary in frequency, phase, timing, amplitude, or offsets. The device 100 can also include power electronics 106 for providing power to the hardware components. For example, the power electronics 106 can include a battery.

The device 100 can include one or more hardware processors 108. The hardware processors 108 can include microcontrollers, digital signal processors, application specific integrated circuit (ASIC), a field programmable gate array (FPGA) or other programmable logic device, discrete gate or transistor logic, discrete hardware components, or any combination thereof designed to perform the functions described herein. In an embodiment, all of the processing discussed herein is performed by the hardware processor(s) 108. The memory 110 can store data specific to patient and rules as discussed below.

In the illustrated figure, the neurostimulation device 100 can include one, two, three, or more sensors 112 which can include any number of combination of inertial measurement units (IMUs) single or multi-axis accelerometers, gyroscopes, inclinometers (to measure and correct for changes in the gravity field resulting from slow changes in the device's orientation), magnetometers; fiber optic electro goniometers, optical tracking or electromagnetic tracking; electromyography (EMG) to detect firing of tremoring muscle; electroneurogram (ENG) signals; cortical recordings by techniques such as electroencephalography (EEG) or direct nerve recordings on an implant in close proximity to the nerve; heart rate or HRV sensors, galvanic skin response sensors (GSR), thermocouples, photoplethysmography sensor (PPG), temperature sensors (e.g., for body/skin temperature or ambient temperature), and/or other physiologic sensors, for example. In some embodiments, the one or more sensors can be employed to measure response to therapy as well as to calibrate therapy. As shown in the figure, the sensor(s) 112 may be optional. In some embodiments, the sensors 112 could include, for example, biomechanical sensors configured to, for example, measure motion, and/or bioelectrical sensors (e.g., EMG, EEG, and/or nerve conduction sensors). Sensors can include, for example, cardiac activity sensors (e.g., ECG, PPG), skin conductance sensors (e.g., galvanic skin response, electrodermal activity), motion sensors (e.g., accelerometers, gyroscopes), and force transducers. The one or more sensors 112 may include an inertial measurement unit (IMU).

In some embodiments, a tremor signal can be calculated based on input from the one or more of the sensors 112. The tremor signal is a representation of the tremulous activity generated in the brain and motor nerves that causes tremulous muscle activation leading to tremor in the hands, head, neck, legs, feet, and vocal cords.

In some embodiments, the sensor (e.g., IMU) 112 can include one or more of a gyroscope, accelerometer, and magnetometer. The sensor 112 can be affixed or integrated with the neuromodulation (e.g., neurostimulation) device 100. In an embodiment, the sensor 112 is an off the shelf component. In addition to its ordinary meaning, the sensor 112 can also include specific components as discussed below. For example, the sensor 112 can include one more sensors capable of collecting motion data. In an embodiment, the sensor 112 includes an accelerometer. In some embodiments, the sensor 112 can include multiple accelerometers to determine motion in multiple axes. Furthermore, the sensor 112 can also include one or more gyroscopes and/or magnetometer in additional embodiments. Since the sensor 112 can be integrated with the neurostimulation device 100, the sensor 112 can generate data from its sensors responsive to motion, movement, or vibration felt by the device 100. Furthermore, when the device 100 with the integrated sensor 112 is worn by a user, the sensor 112 can enable detection of voluntary and/or involuntary motion of the user.

The one or more sensors 112 may include an audio sensor, including but not limited to a microphone, audio transducer, or accelerometer, configured to measure biological processes, such as breathing, talking, or repetitive motion. Sensors, in some embodiments, sense parameters that are used to optimize neurostimulation and facilitate the introduction of variability in stimulation parameter(s) to reduce tolerance and/or habituation to the neurostimulation. As an example, EEG signals, brain activity and/or neuronal activity may be used in this manner. In one embodiment, variation in one or more parameters may be configured/introduced to generate a natural or desired characteristic of brain or neuronal activity over a time period for the treatment of movement, inflammatory, neurological and psychiatric disorders.

The device 100 can optionally include user interface components, such as a feedback generator 114 and a display 116. The display 116 can provide instructions or information to users relating to calibration or therapy. The display 116 can also provide alerts, such an indication of response to therapy, for example. Alerts may also be provided using the feedback generator 114, which can provide haptic feedback to the user, such as upon initiation or termination of stimulation, for reminder alerts, to alert the user of a troubleshooting condition, to perform a tremor inducing activity to measure tremor motion, among others. Accordingly, the user interface components, such as the feedback generator 114 and the display 116 can provide audio, visual, and haptic feedback to the user. In certain embodiments, the feedback generator 114 and/or display 116 is configured for the user to provide satisfaction data to the device 100.

Furthermore, the device 100 can include communications hardware 118 for wireless or wired communication between the device 100 and an external system, such as the user interface device 150 discussed below. The communications hardware 118 can include an antenna. The communications hardware 118 can also include an Ethernet or data bus interface for wired communications.

While the illustrated figure shows several components of the device 100, some of these components are optional and not required in all embodiments of the device 100. In some embodiments, a system can include a diagnostic device or component that does not include neuromodulation functionality. The diagnostic device could be a companion wearable device connected wirelessly through a connected cloud server, and include, for example, sensors such as cardiac activity, skin conductance, and/or motion sensors as described elsewhere herein.

In some embodiments, the device 100 can also be configured to deliver one, two or more of the following: magnetic, vibrational, mechanical, thermal, ultrasonic, or other forms of stimulation instead of, or in addition to electrical stimulation. Such stimulation can be delivered via one, two, or more effectors in contact with, or proximate the skin surface of the patient. However, in some embodiments, the device is configured to only deliver electrical stimulation, and is not configured to deliver one or more of magnetic, vibrational, mechanical, thermal, ultrasonic, or other forms of stimulation.

Although several neurostimulation devices 100 are described herein, in some embodiments nerves are modulated non-invasively to achieve neuro-inhibition. Neuro-inhibition can occur in a variety of ways, including but not limited to hyperpolarizing the neurons to inhibit action potentials and/or depleting neuron ion stores to inhibit firing action potentials. This can occur in some embodiments via, for example, anodal or cathodal stimulation, low frequency stimulation (e.g., less than about 5 Hz, 100 Hz, 150 Hz, 200 Hz, in some cases), or continuous or intermediate burst stimulation (e.g., theta burst stimulation). This can occur in some embodiments via, for example, anodal or cathodal stimulation, high frequency stimulation (e.g., greater than about 1 kHz in some cases), or continuous or intermediate burst stimulation (e.g., theta burst stimulation). In some embodiments, the wearable devices have at least one implantable portion, which may be temporary or more long term. In many embodiments, the devices are entirely wearable and non-implantable.

### User Interface Devices

FIG. 1B illustrates communications between the neurostimulation device 100 and a user interface device 150 over a communication link 130. The communication link 130 can be wired or wireless. The neuromodulation (e.g., neurostimulation) device 100 is capable of communicating and receiving instructions from the user interface device 150. The user interface device 150 can include a computing device. In some embodiments, the user interface device 150 is a mobile computing device, such as a mobile phone, a smartwatch, a tablet, or a wearable computer. The user interface device 150 can also include server computing systems that are remote from the neurostimulation device. In certain embodiments, the user interface device 150 can include a hardware processor(s) 152, a memory 154, a display 156, and power electronics 158. In some embodiments, the user interface device 150 can also include one or more sensors 160, such as sensors described elsewhere herein. Furthermore, in some instances, the user interface device 150 can generate an alert responsive to device issues or a response to therapy. The alert may be received from the neurostimulation device 100 via communication hardware 162.

In additional embodiments, data acquired from the one or more sensors 112 is processed by a combination of the hardware processor(s) 108 and hardware processor(s) 152. In further embodiments, data collected from one or more sensors 112 is transmitted to the user interface device 150 with little or no processing performed by the hardware processors 108. In some embodiments, the user interface device 150 can include a remote server that processes data and transmits signals back to the device 100 (e.g., via the cloud).

FIG. 1C schematically illustrates an embodiment of a neuromodulation assembly 170 and a base station 172. The neuromodulation assembly 170 can include the neurostimulation device 100 (FIG. 1A) and detachable band 174. In some embodiments, the band 174 can include one or more working or stimulating electrodes 102 and one or more counter or common electrodes 102. For example, in some embodiments, the detachable band 174 includes two or more working electrodes 102 (positioned over the median and radial or ulnar nerves) and a counter-electrode positioned on the dorsal side of the wrist. The electrodes 102 could be, for example, dry electrodes or hydrogel electrodes. The base station 172 can be configured to stream movement sensor and usage data on a periodic basis, e.g., daily and charge the neurostimulation device 100. The device stimulation bursting frequency can be calibrated to a lateral postural hold task “wing-beating” or forward postural hold task for a predetermined time, e.g., 5-30 seconds (e.g., 20 seconds) for each subject. Other non-limiting examples of device parameters can be as disclosed elsewhere herein.

Electrical contacts on the device 100 may deliver or transfer electrical or stimulation signals to the electrodes 102 via conductive traces in the band 174. The electrical contacts may be positioned on the bottom surface of the device 100. The device 100 may include one electrical stimulation contact for each stimulating electrode 102 to be applied to the user. The device 100 may include at least one electrical stimulation contact for each nerve that is to be stimulated. For example, the device 100 may include an electrical stimulation contact configured to deliver a signal to the median nerve, the radial nerve, the ulnar nerve or any combination thereof. In some embodiments, the stimulating electrodes 102 are positioned over the median and radial nerves. In some embodiments, the common electrodes 102 are positioned on the dorsal side of the wrist.

In some embodiments, stimulation may alternate between each nerve such that the nerves are not stimulated simultaneously. In some embodiments, all nerves are stimulated simultaneously. In some embodiments, stimulation is delivered to the various nerves with one of many different amplitudes. In some embodiments, stimulation is delivered to the various nerves in one of many bursting patterns. The stimulation parameters may include amplitude, on/off, time duration, intensity, pulse rate, pulse width, waveform shape, and the ramp of pulse on and off. In one embodiment the stimulation may last for approximately 10 minutes to 1 hour, such as approximately 10, 20, 30, 40, 50, or 60 minutes, or ranges including any two of the foregoing values.

The electrodes 102 could be, for example, dry electrodes or hydrogel electrodes. The base station 172 can be configured to stream movement sensor 112 and usage data on a periodic basis, e.g., daily and charge the device 100. The amplitude of the stimulation signal from the device 100 can be varied to increase the efficacy of the treatment. Other non-limiting examples of device 100 parameters can be as disclosed elsewhere herein.

In some embodiments 3-12 or more electrodes are used (e.g., 3, 6, 9 or 12). In one embodiment, none of the electrodes 102 are in contact with areas that cause discomfort. In many embodiments, the transcutaneous device is a wearable band or earpiece. The band may partially or fully surround a wrist, finger, arm, leg, ankle or head. Patches may be used, but in many embodiments a patch is not used.

In some embodiments, stimulation may alternate between each nerve such that the nerves are not stimulated simultaneously. In some embodiments, all nerves are stimulated simultaneously. In some embodiments, stimulation is delivered to the various nerves in one of many bursting patterns. The stimulation parameters may include on/off, time duration, intensity, pulse rate, pulse width, waveform shape, and the ramp of pulse on and off. In one embodiment the pulse rate may be from about 1 to about 5000 Hz, about 1 Hz to about 500 Hz, about 5 Hz to about 50 Hz, about 50 Hz to about 300 Hz, or about 150 Hz, and overlapping ranges therein. In some embodiments, the pulse rate may be from 1 kHz to 20 KHz. In some embodiments, a pulse width may range from, in some cases, 50 to 500 μs (micro-seconds), such as approximately 50-150,150-300, 300-500, such as 100, 200, 300, 400 μs, and overlapping ranges therein. The intensity of the electrical stimulation may vary from 0 mA to 500 mA, and a current may be approximately 1-11, 1-20, 5-50, 10-100 mA, and overlapping ranges therein. The electrical stimulation can be adjusted in different patients and with different methods of electrical stimulation. The increment of intensity adjustment may be, for example, 0.1 mA to 1.0 mA, such as 0.1-0.5, 0.5-0.75, 5-1 mA, and overlapping ranges therein. In some embodiments, the stimulation may last for approximately 10 minutes to 1 hour, such as approximately 10, 20, 30, 40, 50, or 60 minutes, or ranges including any two of the foregoing values. In some embodiments, stimulation may be provided for 30, 40, 50, 60, 80, 90, 120, 150 minutes 1-4 times a day. In some embodiments, stimulation occurs for 2-15 minutes (e.g., 3, 5, 7, 10 minutes) every hour (or on another interval) for a total of 40-240 minutes (e.g., 60, 80, 90, 120, 150 minutes) in a 12 or 24 hour period. Differing dosing schedules and/or differing stimulation parameters may reduce tolerance or habituation and/or may increase patient comfort/compliance. In one embodiment, beneficial effects of stimulation are provided during off periods; for example, a patient's tremor or other symptom/indication is reduced because the prior stimulation results in a prolonged effect on the nerve(s). Thus, a patient may be able to reduce the length, duration etc. of therapy over time. In some embodiments, a plurality of electrical stimuli can be delivered offset in time from each other by a predetermined fraction of multiple of a period of a measured rhythmic biological signal such as hand tremor, such as about ¼, ½, or ¾ of the period of the measured signal for example. Further possible stimulation parameters are described, for example, in U.S. Pat. No. 9,452,287 to Rosenbluth et al., U.S. Pat. No. 9,802,041 to Wong et al., PCT Pub. No. WO 2016/201366 to Wong et al., PCT Pub. No. WO 2017/132067 to Wong et al., PCT Pub. No. WO 2017/023864 to Hamner et al., PCT Pub. No. WO 2017/053847 to Hamner et al., PCT Pub. No. WO 2018/009680 to Wong et al., PCT Pub. No. WO 2018/039458 to Rosenbluth et al., PCT Pub. No. WO 2018/187241 to Hamner et al., PCT Pub. No. WO 2019/213433 to Liberatore et al., PCT Pub. No. WO 2020/006048 to Rosenbluth et al., PCT Pub. No. WO 2020/069219 to Ross et al., PCT Pub. No. WO 2020/086726 to Hamner et al., PCT Pub. No. WO 2020/185601 to Hamner et al., PCT Pub. No. WO 2021/0252278 to Hamner et al., and PCT Pub. No. WO 2021/236815 to Kent et al., each of the foregoing of which are hereby incorporated by reference in their entireties.

In some embodiments, stimulation may be applied to two or more nerves in an alternating manner at an interval defined by the tremor frequency (also referred to as burst frequency). In some embodiments, burst frequency is equal to the measured pathological tremor oscillation, which calculated from measured motion, muscle activity, or brain activity.

Various embodiments of the devices and/or systems discussed herein can stimulate nerves in an outer ear of a user, including but not limited to the auricular branch of the vagus nerve, great auricular nerve, auriculotemporal nerve, and/or lesser occipital nerve, among others. In one embodiment, a system can include a neuromodulation device on the wrist or other location of the arm to target a nerve of a subject (e.g., median nerve) and a neuromodulation device (such as any of the auricular devices described herein) in the ear to target the vagus nerve. In some implementations, each neuromodulation device in the system can communicate with each other via a wired or wireless connection. Multiple neuromodulation devices can provide synchronized stimulation to the multiple nerves. Stimulation may be, for example, burst, offset, or alternating between the multiple nerves. Modulation of the vagus nerve can be accomplished with the devices described herein, according to several embodiments. In some embodiments, the devices described herein are used to stimulate the autonomic system. In some embodiments, the devices described herein are used to balance the sympathetic/parasympathetic systems.

Variability of stimulation parameters, including but not limited to jitter or dither-like variability, can enhance the symptomatic and/or long-term reduction of tremor severity provided by the application of alternating stimulation between two or more peripheral nerves. This approach can overcome the challenge of variability observed in people with hand tremor between tremor episodes within an individual, or the variability observed between people in their brain response to peripheral nerve stimulation. Thus, several embodiments include systems and methods to reduce habituation and/or tolerance to stimulation by, for example, introducing variability in stimulation parameter(s).

Adding variation in burst frequency may account for natural variation in pathological tremor frequency. For example, in some cases pathological tremor frequency can change, for example, by more than 2 Hz between tasks and by up to 32% on the same task over time within an individual subject. Calibrating burst frequency to tremor frequency can improve therapeutic effect. However, as discussed above, it may be difficult to target particular tremor frequencies due to the natural variations. In some instances, it may not be suitable to continuously track the changing tremor characteristics using sensors discussed herein. It may consume too many computational resources and may also deplete battery. Therefore, the inventors realized that instead of focusing on a particular value or trying to exactly align to a pathological characteristic, adding variation in stimulation parameters, such as burst frequency, may enhance therapeutic benefit in treatment of conditions. Pathological characteristics can vary depending on the pathological condition. For example, for treatment of tremor, the characteristics of tremor may include tremor frequency, power, phase, amplitude, and the like. For example, for treatment of migraine, a 3 Hz burst frequency with a 150 Hz pulse frequency may override thalamocortical dysrhythmia in individuals. For example, for treatment of stroke, a 1 Hz burst frequency with a 10 Hz pulse frequency may reduce neuronal inhibition in the motor cortex that otherwise inhibits motor activity in individuals. In some instances, the characteristics may include physiological parameters, such as heart rate, respiration rate and/or content (respiratory rate; respiration phase; capnogram; oximetry; spirography), heart rate variability, blood pressure, and the like. The characteristics may also correspond to sympathetic and/or parasympathetic activity. Furthermore, the characteristics may correspond to neural oscillations. In some instances, neural oscillations may be observed in alpha, beta, delta, theta, gamma frequency bands. In some embodiments, EEG sensor is not required to probe these oscillations and provide therapeutic effect based on stimulation.

In some instances, variations will increase probability of alignment with the changing pathological characteristics during a portion of the therapy session, over time and across tasks. In some embodiments, one or more stimulation parameters are continuously varied over the course of the stimulation. Furthermore, in some instances, measuring tremor characteristics with one or more sensors is not required to provide a therapeutic effect. In addition to tremor, introduction of variability to treat conditions other than tremor are also provided (e.g., other movement disorders, migraine, stroke, other neurological disorders, etc.).

In additional embodiments, stimulation parameters are agnostic for any particular individual and may be varied within generally known therapeutic ranges during the course of stimulation. Adding variation in pulse frequency may account for individual differences in the brain response to peripheral nerve stimulation. For example, the evoked response generated in the ventral intermediate nucleus of the thalamus by median nerve stimulation was maximized at a pulse frequency of 50 Hz in some subjects and 100 Hz in other subjects. By varying pulse frequency throughout these range of values, the brain response is maximized during some portion of the therapy session for every individual, which may enhance therapeutic benefit. Varying pulse frequency during deep brain stimulation (DBS) therapy improved motor score outcomes, gait speed, and freezing of gait episodes in Parkinson's disease patients, compared to fixed frequency DBS. Finally, varying pulse frequency may produce natural stimulation-evoked sensations.

Adding variation in pulse intensity, current amplitude, voltage amplitude, or pulse width would be expected to change the extent of neuronal recruitment within the targeted nerves, with higher intensities and amplitudes, or longer pulse widths, increasing the extent of recruitment. These variations in nerve recruitment may vary the degree of activation in downstream neuronal sub-populations within the brain, which in turn could enhance therapeutic benefit, potentially by reducing the likelihood of neuronal adaptation or habituation to stimulation. In addition, varying pulse intensity or pulse width may produce more natural stimulation-evoked sensations than fixed stimulation. Systems and methods to reduce habituation and/or tolerance to stimulation are provided in several embodiments by, for example, introducing variability in stimulation parameter(s), as described herein. Habituation and/or tolerance to neurostimulation that occur in the treatment of movement, inflammatory, neurological and psychiatric disorders are treated in several embodiments.

Adding on/off periods in the stimulation waveform may enhance the therapeutic effects by increasing the desired desynchronization effect in downstream neuronal sub-populations within the brain.

Additionally, not to be limited by theory, variability in any of the above parameters can enhance the desired neuronal desynchronization effect that enhances therapeutic benefit (e.g., a lower tremor or symptom severity after application of stimulation) and/or comfort.

Variability can be applied to one or more of the following parameters for stimulating a nerve including but not limited to burst frequency or alternating frequency, pulse frequency, pulse width, pulse spacing, intensity, current amplitude, voltage amplitude, duration of stimulation, on/off periods, or amplitude envelope periods. Variability can be applied across multiple stimulation parameters for stimulating a nerve including but not limited to simultaneous variation, braided variation, timescale variation, and adaptive learning. In certain embodiments, adaptive learning is employed in combination with the listed variations as well as other variations to improve neurostimulation therapy outcomes.

### Controllers

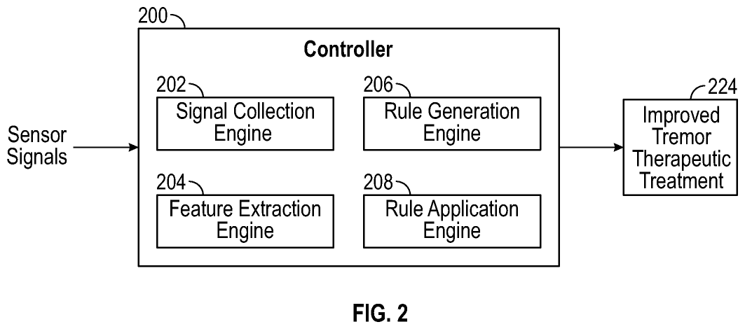

FIG. 2 illustrates a block diagram of an embodiment of a controller 200 that can be implemented with the hardware components described above with respect to FIGS. 1A-1C. The controller 200 can include multiple engines for performing the processes and functions described herein. The engines can include programmed instructions for performing processes as discussed herein for detection of input conditions and control of output conditions. The engines can be executed by the one or more hardware processors of the neuromodulation (e.g., neurostimulation) device 100 alone or in combination with the patient monitor 150. The programming instructions can be stored in a memory 110. The programming instructions can be implemented in C, C++, JAVA, or any other suitable programming languages. In some embodiments, some or all of the portions of the controller 200 including the engines can be implemented in application specific circuitry such as ASICs and FPGAs. Some aspects of the functionality of the controller 200 can be executed remotely on a server (not shown) over a network. While shown as separate engines, the functionality of the engines as discussed below is not necessarily required to be separated. Accordingly, the controller 200 can be implemented with the hardware components described above with respect to FIGS. 1A-1C.

The controller 200 can include a signal collection engine 202. The signal collection engine 202 can enable acquisition of raw data from sensors 112 embedded in the device, including but not limited to accelerometer or gyroscope data from the IMU. In some embodiments, the signal collection engine 202 can also perform signal preprocessing on the raw data. Signal preprocessing can include noise filtering, smoothing, averaging, and other signal preprocessing techniques to clean the raw data. In some embodiments, portions of the signals can be discarded by the signal collection engine 202.

The controller 200 can also include a feature extraction engine 204. The feature extraction engine 204 can extract relevant features from the signals collected by the signal collection engine 202. The features can be in time domain and/or frequency domain. For example, some of the features can include amplitude, bandwidth, area under the curve (e.g., power), energy in frequency bins, peak frequency, ratio between frequency bands, and the like. The features can be extracted using signal processing techniques such as Fourier transform, band pass filtering, low pass filtering, high pass filtering and the like.

The controller 200 can further include a rule generation engine 206. The rule generation engine 206 can use the extracted features from the collected signals and determine rules that correspond to neurostimulation therapy. The rule generation engine 206 can automatically determine a correlation between specific extracted features and neurostimulation therapy outcomes. Outcomes can include, for example, identifying patients who will respond to the therapy (e.g., during an initial trial fitting or calibration process) based on tremor features of kinematic data (e.g., approximate entropy), predicting stimulation settings for a given patient (based on their tremor features) that will result in the best therapeutic effect (e.g., dose, where parameters of the dose or dosing of treatment include but are not limited to duration of stimulation, frequency and/or amplitude of the stimulation waveform, and time of day stimulation is applied), predicting patient tremor severity at a given point, predicting patient response over time, examining patient medication responsiveness combined with tremor severity over time, predicting response to transcutaneous or percutaneous stimulation, or implantable deep brain stimulation or thalamotomy based off of tremor features and severity over time, and predicting ideal time for a patient to receive transcutaneous or percutaneous stimulation, or deep brain stimulation or thalamotomy based off of tremor features and severity over time, predicting patient reported therapy outcomes or patient reported satisfaction using tremor features assessed kinematic measurements from the device; predicting patient response to undesirable user experience using tremor features assessed from kinematic measurements and patient usage logs from the device where undesirable user experiences can include but are not limited to device malfunctions and adverse events such as skin irritation or burn; predict patient response trends based on tremor severity where trends can be assessed across total number of sessions, within an individual patient, or across a population of patients; predicting or classifying subtypes of tremor to predicting patient response based on kinematic analysis of tremor features; predicting or classifying subtypes of tremor to provide guidance for individually optimized therapy parameters; predicting or classifying subtypes of tremor to optimize the future study design based on subtypes (e.g., selecting specific subtypes of essential tremor for a clinical study with specific design addressing therapy need for the subtype); and predict patient or customer satisfaction (e.g., net promoter score) based on patient response or other kinematic features from measure tremor motion. In some embodiments, essential tremor pathology can include, for example, a primarily cerebellar variant with Bergmann gliosis and Purkinje cell torpedoes, and a Lewy body variant, and a dystonic variant, and a multiple sclerosis variant, and a Parkinson disease variant.

In some embodiments, the neuromodulation, e.g., neurostimulation device 100 can apply transcutaneous stimulation to a patient with tremor that is a candidate for implantable deep brain stimulation or thalamotomy. Tremor features and other sensor measurements of tremor severity will be used to assess response over a prespecified usage period, which could be 1 month or 3 months, or 5, 7, 14, 30, 60, or 90 days or more or less. Response to transcutaneous stimulation as assessed, for example, by algorithms described herein using sensor measurements from the device can advantageously provide input to a predictive model that provides an assessment of the patient's likelihood to respond to implantable deep brain stimulation or other implantable or non-implantable therapies.

In some embodiments, the neuromodulation, e.g., neurostimulation device 100 or a secondary device with sensors can collect motion data, or data from other sensors, when a tremor inducing task is being performed. The patient can be directly instructed to perform the task, for example via the display on the device or audio. In some embodiments, features of tremor inducing tasks are stored on the device and used to automatically activate sensors to measure and store data to memory during relevant tremor tasks. The period of time for measuring and storing data can be, for example, 10, 20, 30, 60, 90, 120 seconds, or 1, 2, 3, 5, 10, 15, 20, 30 minutes, or 1, 2, 3, 4, 5, 6, 7, 8 hours or more or less, or ranges incorporating any two of the foregoing values. Based on a training set of data from a cohort of previous wearers with tremor or another condition, the feature extraction engine can detect features that are correlated with response to stimulation such that the patient or physician can be presented with a quantitative and/or qualitative likelihood of the patient responding or not responding to treatment. This data can be measured in some cases prior to prescribing the neuromodulation, e.g., neurostimulation or during a trial period.

In another embodiment, features can be correlated with the type of tremor measured, such as resting tremor (associated with Parkinson's Disease), postural tremor, action tremor, intention tremor, rhythmic tremor (e.g., a single dominant frequency) or mixed tremor (e.g., multiple frequencies). The type of tremor most likely detected can be presented to the patient or physician as a diagnosis or informative assessment prior to receiving stimulation or to assess appropriateness of prescribing a neuromodulation, e.g., stimulation treatment. In another embodiment, various stimulation modes may be applied based on the tremor type determined; different modes could include changes in stimulation parameters, such as frequency, pulse width, amplitude, burst frequency, duration of stimulation, or time of day stimulation is applied. In one embodiment for a smartphone, tablet, or other device 150, the task to induce tremor can be included in an app that asks the patient to take a self-photograph, which has the patient perform a task that has both posture and intention actions.

In some embodiments, the neuromodulation, e.g., neurostimulation device 100 or a secondary device with sensors can collect motion data, or data from other sensors, can measure data over a longer period of time, for example 1, 2, 3, 4, 5, 10, 20, 30 weeks, 1, 2, 3, 6, 9, 12 months, or 1, 2, 3, 5, 10 years or more or less, or ranges incorporating any two of the foregoing values, to determine features, or biomarkers, associated with the onset of tremor diseases, such as essential tremor, Parkinson's disease, dystonia, multiple sclerosis, etc. Biomarkers could include specific changes in one or more features of the data over time, or one or more features crossing a predetermined threshold. In some embodiments, features of tremor inducing tasks have been stored on the device and used to automatically activate sensors when those tremor inducing tasks are being performed, to measure and store data to memory during relevant times.

In some embodiments, the rule generation engine 206 relies on instructions to determine rules between features and outcomes. The rule generation engine 206 can employ machine learning modeling along with signal processing techniques to determine rules, where machine learning modeling and signal processing techniques include but are not limited to: supervised and unsupervised algorithms for regression and classification. Specific classes of algorithms include, for example, Artificial Neural Networks (Perceptron, Back-Propagation, Convolutional Neural Networks, Recurrent Neural networks, Long Short-Term Memory Networks, Deep Belief Networks), Bayesian (Naive Bayes, Multinomial Bayes and Bayesian Networks), clustering (k-means, Expectation Maximization and Hierarchical Clustering), ensemble methods (Classification and Regression Tree variants and Boosting), instance-based (k-Nearest Neighbor, Self-Organizing Maps and Support Vector Machines), regularization (Elastic Net, Ridge Regression and Least Absolute Shrinkage Selection Operator), and dimensionality reduction (Principal Component Analysis variants, Multidimensional Scaling, Discriminant Analysis variants and Factor Analysis). In some embodiments, the controller 200 can use the rules to automatically determine outcomes. The controller 200 can also use the rules to control or change settings of the neurostimulation device, including but not limited to stimulation parameters (e.g., stimulation amplitude, frequency, patterned (e.g., burst stimulation), intervals, time of day, individual session or cumulative on time, and the like).

Accordingly, the rules can improve operation of the neuromodulation, e.g., neurostimulation device 100, and advantageously and accurately identify potential candidates for therapy and well as various disease state and therapy parameters over time.

The neuromodulation devices, e.g., neurostimulation devices, described herein, in several embodiments, can be used for the treatment of depression (including but not limited to post-partum depression, depression affiliated with neurological diseases, major depression, seasonal affective disorder, depressive disorders, etc.), inflammation (e.g., neuroinflammation), Lyme disease, stroke, neurological diseases (such as Parkinson's and Alzheimer's), and gastrointestinal issues (including those in Parkinson's disease). The devices described herein may also be used for the treatment of inflammatory bowel disease (such as Crohn's disease, colitis, and functional dyspepsia), rheumatoid arthritis, multiple sclerosis, psoriatic arthritis, osteoarthritis, psoriasis and other inflammatory diseases. The devices described herein can be used for the treatment of inflammatory skin conditions in some embodiments. The neuromodulation devices, e.g., neurostimulation devices, described herein can be used for the treatment of chronic fatigue syndrome. The devices described herein can be used for the treatment of chronic inflammatory symptoms and flare ups. Bradykinesia, dyskinesia, rigidity may also be treated according to several embodiments. In several embodiments, rehabilitation as a result of certain events is treated, for example, rehabilitation from stroke or other cardiovascular events. In several embodiments, treatment of involuntary and/or repetitive movements is provided, including but not limited to tics, twitches, etc. (including, for example, Tourette Syndrome, tic disorders). Rhythmic and non-rhythmic involuntary movements may be controlled in several embodiments. Involuntary vocal tics and other vocalizations may also be treated. Systems and methods to reduce habituation and/or tolerance to stimulation in the disorders and symptoms identified herein are provided in several embodiments by, for example, introducing variability in stimulation parameter(s) described herein.

In several embodiments, the neuromodulation, e.g., neurostimulation, devices described herein can be used for the treatment of cardiac conditions (such as atrial fibrillation, hypertension, and stroke), and for the treatment of immune dysfunction. Epilepsy is treated in one embodiment.

In some embodiments, provided herein are treatments of restless leg syndrome, periodic limb movement disorder, repetitive movements of the limbs and abnormal sensation. Devices described herein can be placed, for example, on the wrist or leg (or both) to treat limb disorders. In some embodiments, vagus nerve stimulation is used to treat restless leg syndrome, periodic limb movement disorder, repetitive movements of the limbs and/or abnormal limb sensation. With respect to the leg, a device may be placed, for example, on the thigh, calf, ankle or other location suitable to treat the target nerve(s). The devices described herein can be used to stimulate the autonomic nervous system. The devices described herein can be used to balance the sympathetic/parasympathetic nervous systems. Dysfunction or imbalance of the autonomic nervous system is believed to be a potential underlying mechanism for various chronic diseases. Autonomic dysfunction can develop when the nerves of the ANS are damaged or degraded or without any known neural pathology. This condition is called autonomic neuropathy or dysautonomia. Autonomic dysfunction can range from mild to life-threatening and can affect part of the ANS or the entire ANS. Sometimes the conditions that cause problems are temporary and reversible. Others are chronic, or long term, and may continue to worsen over time. Examples of chronic diseases that are associated with autonomic dysfunction include, but are not limited to, diabetes, Parkinson's disease, tremor, cardiac arrhythmias including atrial fibrillation, hypertension, overactive bladder, urinary incontinence, fecal incontinence, inflammatory bowel diseases, rheumatoid arthritis, migraine, depression, and anxiety.

In some embodiments, disorders and symptoms caused or exacerbated by microbial infections (e.g., bacteria, viruses, fungi, and parasites) are treated. Symptoms include but are not limited to sympathetic/parasympathetic imbalance, autonomic dysfunction, inflammation (including but not limited to neuroinflammation and other inflammation), motor and balance dysfunction, pain and other neurological symptoms. Disorders include but are not limited to tetanus, meningitis, Lyme disease, urinary tract infection, mononucleosis, chronic fatigue syndrome, autoimmune disorders, etc. In some embodiments, autoimmune disorders and/or pain unrelated to microbial infection is treated, including for example, inflammation (e.g., neuroinflammation, etc.), headache, back pain, joint pain and stiffness, muscle pain and tension, etc. Other disorders (e.g., hypertension, dexterity, and cardiac dysrhythmias) can also be treated using the embodiments described herein.

In some embodiments, modulation of the blood vessel (either dilation or constriction) is provided using the devices and methods described herein (e.g., through nerve stimulation). Such therapy may, in turn, reduce inflammation (including but not limited to inflammation post microbial infection). The devices and methods described herein increase, decrease or otherwise balance vasodilation and vasoconstriction through neuromodulation in some embodiments. For example, reduction of vasodilation is provided in several embodiments to treat or prevent migraine or other conditions that are aggravated by vasodilation. In other embodiments, vasoconstriction is reduced in, for example, conditions in which dilation is beneficial (such as with high blood pressure and pain). In one embodiment, reduction in inflammation treats tinnitus. In some embodiments, modulation of the blood vessel (either dilation or constriction) is used to treat tinnitus. Tinnitus may be treated according to several embodiments through modulation (e.g., stimulation) of the vagus nerve alone or in conjunction with one, two or more other nerves (including for example the trigeminal nerve, great auricular nerve, nerves of the auricular branch, auricular branch of the vagus nerve, facial nerve, the auriculotemporal nerve, etc.). In one embodiment, nerves other than the vagus nerve are modulated to treat tinnitus. Cranial/auditory nerves may be modulated to treat tinnitus and/or auricular inflammation in some embodiments. Auricular devices may be used in conjunction with devices placed on limbs to in some embodiments (e.g., an ear device along with a wrist device).

The generated rules can be saved in the memory 110 and/or memory 154. For example, the rules can be generated and stored prior to operation of the neurostimulation device 100. Accordingly, in some embodiments, a rule application engine 208 can apply the saved rules on new data collected by the sensors 112 to determine outcomes or control the neuromodulation, e.g., neurostimulation device 100. The controller 200 can select from methods for varying parameter(s) employed during therapy session to improve tremor therapeutic treatment 224.

In some embodiments, the neuromodulation device 100 can include the ability to track a user's motion data for the purpose of gauging one, two, or more tremor frequencies of a patient. The patient could have a single tremor frequency, or in some cases multiple discrete tremor frequencies that manifest when performing different tasks. Once the tremor frequencies are observed, they can be used as one of many seminal input parameters to a customized neuromodulation therapy. The therapy can be delivered, e.g., transcutaneously, via one, two, or more nerves (e.g., the median and radial or ulnar nerves, and/or other nerves disclosed elsewhere herein) in order to reduce or improve a condition of the patient, including but not limited to their tremor burden. In some embodiments, the therapy modulates afferent nerves, but not efferent nerves. In some embodiments, the therapy preferentially modulates afferent nerves. In some embodiments, the therapy does not involve functional electrical stimulation. The tremor frequency can be used to calibrate the patient's neuromodulation therapy, being used as a calibration frequency in some embodiments to set one or more parameters of the neuromodulation therapy, e.g., a burst envelope period. In some embodiments, the calibration frequency can be between, for example, about 4 Hz and about 12 Hz, between about 3 Hz and about 6 Hz, or about 3 Hz, 4 Hz, 5 Hz, 6 Hz, 7 Hz, 8 Hz, 9 Hz, 10 Hz, 11 Hz, or 12 Hz, or ranges including any two of the foregoing values. Specific examples for controlling the neurostimulation device 100 are described in more detail below.

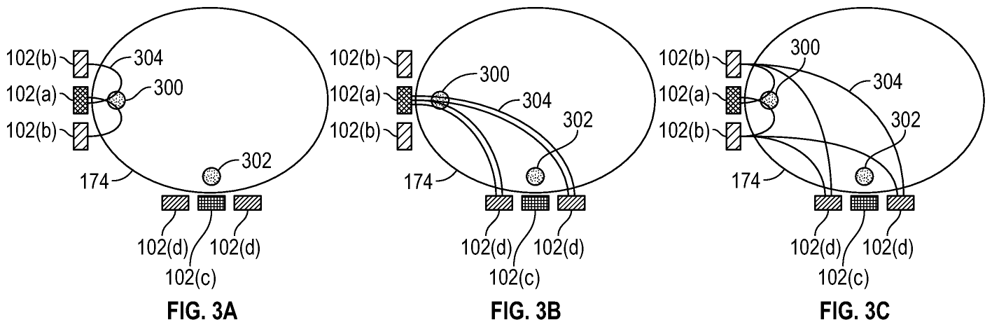

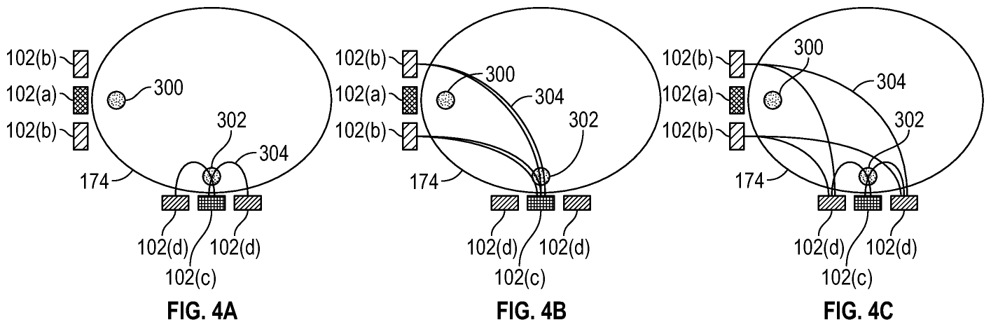

In some embodiments, the device 100 activates the nerves in different anatomical ways to increase therapy variability in the areas of the nerves. For example, in some embodiments, multiple stimulation channels can be used to activate different areas of primary and secondary target nerves and create a more durable benefit for the user. In some embodiments, the device 100 couples one stimulation channel to one nerve output. In some embodiments, the device 100 couples one stimulation channel to “uncoupled commons” located in closer proximity to the primary target nerve than to a secondary target nerve. In some embodiments, the device 100 couples one stimulation channel to “opposite commons” located in closer proximity to the secondary target nerve than to the primary target nerve. In some embodiments, the device 100 couples one stimulation channel to “coupled commons” located in proximity to both the primary target nerve and the secondary target nerve. In this way, in some embodiments, the potential number of stimulation channels at the wrist is increased. In some embodiments, each stimulation channel can include one or more stimulation current paths 304 (FIG. 3A). An increase in the potential number of stimulation channels can generate different individual and combinations of nerve stimulation as shown by the variations in the stimulation current paths 304 in FIGS. 3 and 4.

FIGS. 3A-3C illustrate three activation embodiments where the common electrodes 102(b), 102(d) of the band 174 are positioned near the radial nerve 300 and the medial nerve 302, respectively. In some embodiments, the primary target nerve for non-invasive electrical stimulation is the radial nerve 300. Of course, the positioning of the common electrodes 102(b), 102(d) is only exemplary. The common electrodes 102(b), 102(d) can be placed at any circumferential locations about the band 174.

Non-invasive electrical stimulation of peripheral nerves, e.g., in the wrist, including the median 302 and radial 300 nerves, can result in decreased hand tremor in users with essential tremor. Motion sensors 112 placed on the wrist can quantify tremor and provide kinematic measures that correlate with clinical ratings of tremor severity. Tremor severity can be quantified with wearable wrist sensor measurements during clinical ratings of hand tremor before and at distinct timepoints (e.g., up to 60 minutes or more) following a single session of non-invasive median and radial or ulnar nerve stimulation. In one embodiment, the median nerve is stimulated along with either the radial or ulnar nerve only in one or more stimulation sessions.

In some embodiments, the band 174 (FIG. 1C) can include one or more working or stimulating electrodes 102(a), 102(c) and one or more counter or common electrodes 102(b), 102(d). In some embodiments, the device 100 includes at least three electrodes, e.g., hydrogel electrodes (or dry electrodes in other embodiments) positioned transcutaneously to target the median 302 and radial 300 nerves of each user. For example, in some embodiments, the device 100 includes a first (e.g., median) electrode 102(c), a second (e.g., radial) electrode 102(a), and one or more third (e.g., counter) electrodes 102(b), 102(d) placed circumferentially on the band 174.

In some embodiments, the band 174 can have more or fewer electrodes 102(b), 102(d) than the number of stimulating electrodes 102(a), 102(c). In some embodiments, the band 174 can have one common electrode 102(b), 102(d) for each stimulating electrode 102(a), 102(c). In certain other embodiments, the band 174 can have more than one common electrode 102(b), 102(d) for each stimulating electrode 102(a), 102(c). In certain other embodiments, the band 174 can have more than one stimulating electrode 102(a), 102(c) for each common electrode 102(b), 102(d). In the illustrated embodiment, the common electrodes 102(b) are positioned on opposite sides of the stimulating electrode 102(a) on the band 174. In the illustrated embodiment, the common electrodes 102(d) are positioned on opposite sides of the stimulating electrode 102(c) on the band 174. In some embodiments, the stimulating electrode 102 is spaced 90 degrees to 180 degrees from an adjacent stimulating electrode 102 as measured along a circumference of the band 174. In some embodiments, the stimulating electrode 102 is spaced 0 degrees to 30 degrees from an adjacent common electrode 102. Of course, the disclosure is not limited to the listed spacings. Accordingly, any other spacing falls within the scope of the disclosure, e.g., 0 degrees to 15 degrees, 15 degrees to 30 degrees, 15 degrees to 45 degrees, etc.

Electrical contacts on the neuromodulation assembly 170 (FIG. 1C) may deliver or transfer electrical or stimulation signals to the electrodes 102 via conductive traces in the band 174. The electrical contacts may be positioned on the bottom surface of the device 100 at a location where the device 100 interfaces with the band 174. In some embodiments, the device 100 may include one electrical stimulation contact for each stimulating electrode 102(a), 102(c) to be applied to the user. In some embodiments, the device 100 may include at least one electrical stimulation contact for each nerve 300, 302 that is to be stimulated. For example, the neuromodulation assembly 170 may include an electrical stimulation contact configured to deliver a signal to the median nerve 302, the radial nerve 300, the ulnar nerve, or any combination thereof. In some embodiments, the stimulating electrodes 102(a), 102(c) are positioned over the median nerve 302 and the radial nerve 300.

In the embodiments illustrated in FIGS. 3A-3C, the common electrodes 102(b), 102(d) are positioned on both sides of each stimulating electrode 102(a), 102(c). Of course, the number and arrangement of the electrodes 102 on the band 174 are not limited to the illustrated embodiments. The band 174 can have any number of electrodes 102 positioned along the band 174.

Referring to FIGS. 3A-3C, one or more stimulation current paths 304 can be created between a stimulating electrode 102(a), 102(c) and a common electrode 102(b), 102(d) when the device 100 activates one or more stimulating electrode 102(a), 102(c) and activates one or more common electrodes 102(b), 102(d) during a first time frame. During a second time frame, the device 100 can maintain or change the state of the one or more stimulating electrodes 102(a), 102(c) and/or the one or more common electrodes 102(b), 102(d). In this way, the path 304 and/or number of the one or more stimulation current paths 304 created by the device 100 during the first time frame change during the second time frame.

In some embodiments, the device 100 can change a parameter of a stimulation signal sent to the one or more stimulating electrodes 102(a), 102(c) between the first and second time frames to create the one or more stimulation channels. Parameters of the stimulation signal can include (e.g., stimulation amplitude, frequency, patterned (e.g., burst stimulation), intervals, time of day, individual session or cumulative on time, and the like).

In some embodiments, the device 100 can change the parameter of the stimulation signal as well as the state of the one or more stimulating electrodes 102(a), 102(c) and/or the one or more common electrodes 102(b), 102(d) between the first and second time frames. In the illustrated embodiments of FIGS. 3A-3C, the common electrodes 102(b), 102(d) are selectively activated/deactivated to change the path 304 and/or number of the one or more stimulation current paths 304 from the working electrode 102(a) positioned near the radial nerve 300.

For example, in the illustrated embodiment of FIG. 3A, the device 100 activates the common electrodes 102(b) to create multiple stimulation current paths 304 between the stimulating electrode 102(a) and the common electrodes 102(b). The radial electrode 102(a) and the common electrodes 102(b) are activated in FIG. 3A while the common electrodes 102(d) are deactivated. In this way, the stimulation current paths 304 of the stimulation channel flow between the stimulating electrode 102(a) and the common electrodes 102(b). The common electrodes 102(d) are uncoupled relative to the stimulating electrode 102(a). While the stimulation current paths 304 of the stimulation channel in FIG. 3A are illustrated as having U-shapes, the stimulation current paths 304 can have any shape. Further, the number of stimulation current paths 304 are only exemplary in FIG. 3A. The number of stimulation current paths 304 can be more or less than the number of stimulation current paths 304 illustrated in FIG. 3A.

A level of activation of the one or more nerve 300, 302 caused by the one or more stimulation channels 304 can vary depending on, for example, the proximity of the nerve 300, 302 to the one or more stimulation current paths 304. Further, the level of activation of each nerve 300, 302 caused by the one or more stimulation current paths 304 can vary depending on the parameters of the stimulation signal. For example, in the embodiment illustrated in FIG. 3A, the level of activation of the radial nerve 300 can be different than a level of activation of the median nerve 302. In some embodiments, the level of activation of the radial nerve 300 is greater than a level of activation of the median nerve 302. In some embodiments, an amplitude of the stimulation signal can be selected to increase or decrease the level of activation of the radial nerve 300 as well as the level of activation of the median nerve 302.

In the illustrated embodiment of FIG. 3B, the device 100 activates the common electrodes 102(d) to create multiple stimulation current paths 304 between the stimulating electrode 102(a) and the common electrodes 102(d). The radial electrode 102(a) and the common electrodes 102(d) are activated in FIG. 3B while the common electrodes 102(b) are deactivated. In this way, the stimulation current paths 304 of the stimulation channel flow between the stimulating electrode 102(a) and the common electrodes 102(d). The common electrodes 102(d) are coupled relative to the stimulating electrode 102(a). While the stimulation current paths 304 of the stimulations channel in FIG. 3B are illustrated as having a specific shape, the stimulation current paths 304 can have any shape.

In the embodiment illustrated in FIG. 3B, the level of activation of the radial nerve 300 can be different than a level of activation of the median nerve 302. In some embodiments, the level of activation of the radial nerve 300 is greater than a level of activation of the median nerve 302. In some embodiments, an amplitude of the stimulation signal can be selected to increase or decrease the level of activation of the radial nerve 300 as well as the level of activation of the median nerve 302.

In the illustrated embodiment of FIG. 3C, the device 100 activates the common electrodes 102(b), 102(d) to create multiple stimulation current paths 304 between the stimulating electrode 102(a) and the common electrodes 102(b), 102(d). The radial electrode 102(a) and the common electrodes 102(b), 102(d) are activated in FIG. 3C. In this way, the stimulation current paths 304 of the stimulation channel flow from the stimulating electrode 102(a) to the common electrodes 102(b), 102(d). The common electrodes 102(b), 102(d) are coupled relative to the stimulating electrode 102(a). While the stimulation current paths 304 of the stimulation channel in FIG. 3C are illustrated as having a specific shape, the stimulation current paths can have any shape.

In the embodiment illustrated in FIG. 3C, the level of activation of the radial nerve 300 can be different than a level of activation of the median nerve 302. In some embodiments, the level of activation of the radial nerve 300 is greater than a level of activation of the median nerve 302. In some embodiments, an amplitude of the stimulation signal can be selected to increase or decrease the level of activation of the radial nerve 300 as well as the level of activation of the median nerve 302.

FIGS. 4A-4C illustrate three stimulation channels where the common electrodes 102(b), 102(d) of the band 174 are positioned near the radial nerve 300 and the medial nerve 302, respectively. In some embodiments, the primary target nerve is the median nerve 302. Of course, the positioning of the common electrodes 102(b), 102(d) is only exemplary. The common electrodes 102(b), 102(d) can be placed at any circumferential locations about the band 174.

One or more stimulation current paths 304 can be created between the stimulating electrode 102(a), 102(c) and the common electrode 102(b), 102(d) when the device 100 activates one or more stimulating electrode 102(a), 102(c) and activates one or more common electrodes 102(b), 102(d) during a first time frame. During a second time frame, the device 100 can maintain or change the state of the one or more stimulating electrodes 102(a), 102(c) and/or the one or more common electrodes 102(b), 102(d). In this way, the path 304 and/or number of the one or more stimulation current paths 304 created by the device 100 during the first time frame change during the second time frame.

In some embodiments, the device 100 can change a parameter of a stimulation signal sent to the one or more stimulating electrodes 102(a), 102(c) between the first and second time frames to create the one or more stimulation channels 304. Parameters of the stimulation signal can include (e.g., stimulation amplitude, frequency, patterned (e.g., burst stimulation), intervals, time of day, individual session or cumulative on time, and the like).

In some embodiments, the device 100 can change the parameter of the stimulation signal as well as the state of the one or more stimulating electrodes 102(a), 102(c) and/or the one or more common electrodes 102(b), 102(d) between the first and second time frames. In the illustrated embodiments of FIGS. 4A-4C, the common electrodes 102(b), 102(d) are selectively activated/deactivated to change the path 304 and/or number of the one or more stimulation current paths 304 from the working electrode 102(c) positioned near the median nerve 302.

For example, in the illustrated embodiment of FIG. 4A, the device 100 activates the common electrodes 102(d) to create multiple stimulation current paths 304 between the stimulating electrode 1021 and the common electrodes 102(d). The median electrode 102(c) and the common electrodes 102(d) are activated in FIG. 4A while the common electrodes 102(b) are deactivated. In this way, the stimulation current paths 304 of the stimulation channel flow between the stimulating electrode 102(c) and the common electrodes 102(d). The common electrodes 102(b) are uncoupled relative to the stimulating electrode 102(c). While the stimulation current paths 304 of the stimulation channel in FIG. 4A are illustrated as having U-shapes, the stimulation current paths 304 can have any shape. Further, the number of stimulation current paths 304 are only exemplary in FIG. 4A. The number of stimulation current paths 304 can be more or less than the number of stimulation current paths 304 illustrated in FIG. 4A.

A level of activation of the one or more nerve 300, 302 caused by the one or more stimulation channels 304 can vary depending on, for example, the proximity of the nerve 300, 302 to the one or more stimulation current paths 304. Further, the level of activation of each nerve 300, 302 caused by the one or more stimulation current paths 304 can vary depending on the parameters of the stimulation signal. For example, in the embodiment illustrated in FIG. 4A, the level of activation of the radial nerve 300 can be different than a level of activation of the median nerve 302. In some embodiments, the level of activation of the median nerve 302 is greater than a level of activation of the radial nerve 300. In some embodiments, an amplitude of the stimulation signal can be selected to increase or decrease the level of activation of the median nerve 302 as well as the level of activation of the radial nerve 300.

In the illustrated embodiment of FIG. 4B, the device 100 activates the common electrodes 102(b) to create multiple stimulation current paths 304 between the stimulating electrode 102(c) and the common electrodes 102(b). The radial electrode 102(c) and the common electrodes 102(b) are activated in FIG. 4B while the common electrodes 102(d) are deactivated. In this way, the stimulation current paths 304 of the stimulation channel flow between the stimulating electrode 102(c) and the common electrodes 102(b). The common electrodes 102(b) are coupled relative to the stimulating electrode 102(c). While the stimulation current paths 304 of the stimulation channel in FIG. 4B are illustrated as having a specific shape, the stimulation current paths 304 can have any shape.

In the embodiment illustrated in FIG. 4B, the level of activation of the radial nerve 300 can be different than a level of activation of the median nerve 302. In some embodiments, the level of activation of the median nerve 302 is greater than a level of activation of the radial nerve 300. In some embodiments, an amplitude of the stimulation signal can be selected to increase or decrease the level of activation of the radial nerve 300 as well as the level of activation of the median nerve 302.

In the illustrated embodiment of FIG. 4C, the device 100 activates the common electrodes 102(b), 102(d) to create multiple stimulation current paths 304 between the stimulating electrode 102(c) and the common electrodes 102(b), 102(d). The median electrode 102(c) and the common electrodes 102(b), 102(d) are activated in FIG. 4C. In this way, the stimulation current paths 304 of the stimulation channel flow from the stimulating electrode 102(c) to the common electrodes 102(b), 102(d). The common electrodes 102(b), 102(d) are coupled relative to the stimulating electrode 102(c). While the stimulation current paths 304 of the stimulation channels in FIG. 4C are illustrated as having a specific shape, the stimulation current paths 304 can have any shape.

In the embodiment illustrated in FIG. 4C, the level of activation of the median nerve 302 can be different than a level of activation of the radial nerve 300. In some embodiments, the level of activation of the median nerve 302 is greater than a level of activation of the radial nerve 300. In some embodiments, an amplitude of the stimulation signal can be selected to increase or decrease the level of activation of the median nerve 302 as well as the level of activation of the radial nerve 300.

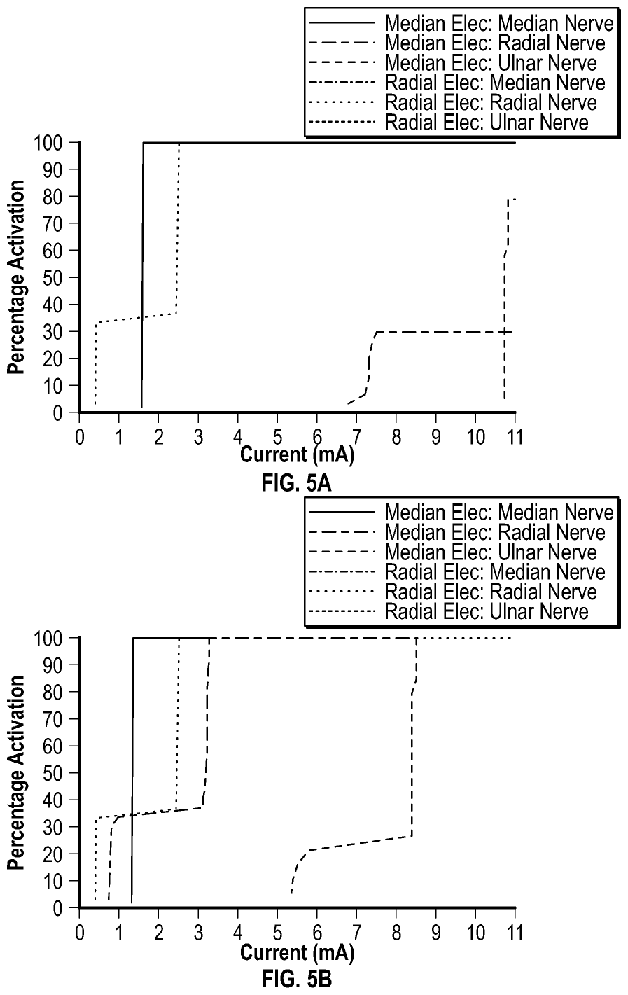

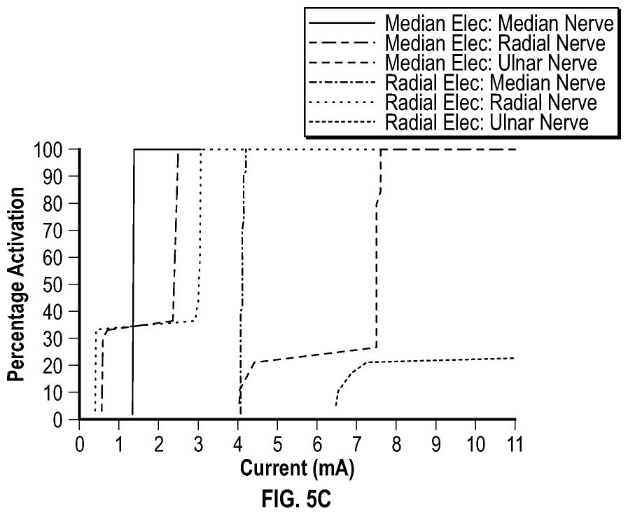

FIGS. 5A-5C includes nerve stimulation plots of percentage activation v. current for the embodiments illustrated in FIGS. 3A-3C and 4A-4C. The plots show different patterns of nerve activation for the radial, median, and ulnar nerves for each of the embodiments illustrated in FIGS. 3A-3C and 4A-4C. As used herein to describe FIGS. 5A-5C, the terms low, medium, and high are ranges of 0-4 mA, 4-8 mA, and higher than 8 mA, respectively.

FIG. 5A corresponds to the “uncoupled commons” embodiments illustrated in FIGS. 3A and 4A. For example, the radial plots in FIG. 5A correspond to the device 100 activating the common electrodes 102(b) to create multiple stimulation current paths 304 between the stimulating electrode 102(a) and the common electrodes 102(b) in FIG. 3A. The radial electrode 102(a) and the common electrodes 102(b) are activated while the common electrodes 102(d) are deactivated. In this way, the stimulation current paths 304 of the stimulation channel flow between the stimulating electrode 102(a) and the common electrodes 102(b). As is illustrated in FIG. 5A, only the radial nerve 300 is activated when a low or high current stimulation signal is applied by the device 100. The median nerve 302 and the ulnar nerves were not measurably activated. The period of time for measuring and storing data can be, for example, 10, 20, 30, 60, 90, 120 seconds, or 1, 2, 3, 5, 10, 15, 20, 30 minutes, or 1, 2, 3, 4, 5, 6, 7, 8 hours or more or less, or ranges incorporating any two of the foregoing values.

The median plots in FIG. 5A correspond to the device 100 activating the common electrodes 102(d) to create multiple stimulation current paths 304 between the stimulating electrode 102(c) and the common electrodes 102(d) in FIG. 4A. The median electrode 102(c) and the common electrodes 102(d) are activated while the common electrodes 102(b) are deactivated. In this way, the stimulation current paths 304 flow between the stimulating electrode 102(c) and the common electrodes 102(d). As is illustrated in FIG. 5A, the median nerve 302 is activated when a low or high current stimulation signal is applied by the device 100. The radial nerve 300 was also activated in response to a medium current stimulation signal being applied by the device 100. The ulnar nerve was only activated in response to a high current stimulation signal being applied by the device 100.

FIG. 5B corresponds to the “coupled commons” embodiments illustrated in FIGS. 3C and 4C. For example, the radial plots in FIG. 5B correspond to the device 100 activating the common electrodes 102(b), 102(d) to create multiple stimulation current paths 304 between the stimulating electrode 102(a) and the common electrodes 102(b), 102(d) in FIG. 3C. The radial electrode 102(a) and the common electrodes 102(b), 102(d) are activated. In this way, the stimulation current paths 304 of the stimulation channel flow between the stimulating electrode 102(a) and the common electrodes 102(b), 102(d). As is illustrated in FIG. 5B, only the radial nerve 300 is activated when a low or high current stimulation signal is applied by the device 100. The median nerve 302 and the ulnar nerves were not measurably activated.

The median plots in FIG. 5B correspond to the device 100 activating the common electrodes 102(b), 102(d) to create multiple stimulation current paths 304 between the stimulating electrode 102(c) and the common electrodes 102(b), 102(d) in FIG. 4C. The median electrode 102(c) and the common electrodes 102(b), 102(d) are activated. In this way, the stimulation current paths 304 flow between the stimulating electrode 102(c) and the common electrodes 102(b), 102(d). As is illustrated in FIG. 5B, the median nerve 302 is activated when a low or high current stimulation signal is applied by the device 100. The radial nerve 300 was also activated in response to a low current stimulation signal being applied by the device 100. The ulnar nerve was activated in response to a medium current stimulation signal being applied by the device 100.

FIG. 5C corresponds to the “opposite commons” embodiments illustrated in FIGS. 3B and 4B. For example, the radial plots in FIG. 5C correspond to the device 100 activating the common electrodes 102(d) to create multiple stimulation current paths 304 between the stimulating electrode 102(a) and the common electrodes 102(d) in FIG. 3B. The radial electrode 102(a) and the common electrodes 102(d) are activated while the common electrodes 102(b) are deactivated. In this way, the stimulation current paths 304 of the stimulation channel flow between the stimulating electrode 102(a) and the common electrodes 102(d). As is illustrated in FIG. 5C, the radial nerve 300 is activated when a low current stimulation signal is applied by the device 100. The median nerve 302 and the ulnar nerves were activated in response to a medium current stimulation signal being applied by the device 100.

The median plots in FIG. 5C correspond to the device 100 activating the common electrodes 102(b) to create multiple stimulation current paths 304 between the stimulating electrode 102(c) and the common electrodes 102(b) in FIG. 4B. The median electrode 102(c) and the common electrodes 102(b) are activated while the common electrodes 102(d) are deactivated. In this way, the stimulation current paths 304 of the stimulation channel flow between the stimulating electrode 102(c) and the common electrodes 102(b). As is illustrated in FIG. 5C, the median nerve 302 is activated when a low or high current stimulation signal is applied by the device 100. The radial nerve 300 was also activated in response to a low current stimulation signal being applied by the device 100. The ulnar nerve was activated in response to a medium current stimulation signal being applied by the device 100.

A comparison of the plots from FIGS. 5A-5C indicates the device 100 can activate different combinations of nerves (e.g., primary target nerve, secondary target nerve, and/or tertiary nerve target) as well as activate each nerve to different activation levels by selectively activating/deactivating certain electrodes 102. Leveraging different arrangements of common electrodes 102(b), 102(d) (e.g., uncoupled, coupled, and opposite commons) increases the number of stimulation channels. In some embodiments, the increased number of stimulation channels can then be used in an algorithm to increase variability in nerve activation (e.g., activation of different nerve branches). In this way, in some embodiments, the potential number of stimulation channels at the wrist is increased. The increase in the potential number of stimulation channels can generate different individual and combinations of nerve stimulation.

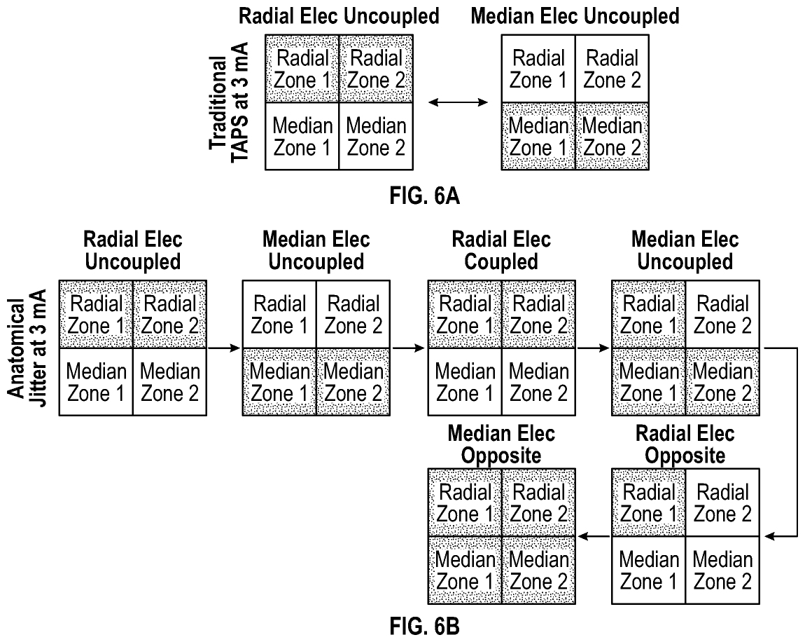

FIG. 6 represents stimulation patterns of stimulation channels in FIG. 6A and FIG. 6B. The pattern illustrated in FIG. 6A alternates between two different stimulation channels (e.g., activation/deactivation configurations of common electrodes 102(b), 102(d)) to change the path 304 and/or number of the stimulation current paths 304 in the wrist. The pattern illustrated in FIG. 6B cycles through six different activation/deactivation configurations of common electrodes 102(b) 102(d) to change the path and/or number of the one or more stimulation channels 304 in the wrist. Of course, this disclosure is not limited to the patterns illustrated in FIGS. 6A and 6B. In some embodiments, any one or more of the activation/deactivation configurations of common electrodes 102(b), 102(d) illustrated in FIG. 6 can be combined/duplicated in any way to create a pattern. In this way, the resulting pattern can include any number of activation/deactivation configurations of common electrodes 102(b) 102(d).

In some embodiments, the patterns illustrated in FIG. 6 can be implemented during one or more therapy sessions. For example, in some embodiments, the controller 200 alternates between the different stimulation channels during all therapy sessions. In some embodiments, the controller 200 performs an initial “run in period.” During the “run in period”, the controller 200 can take into account, for example, typical placement of the band 174 over time, stimulation amplitude, alternating patterns of stimulation, and improvement over a series of therapy sessions to then determine an appropriate nerve patterning that has improved efficacy for the user. In some embodiments, the controller 200 includes one or more preset stimulation patterns. In some embodiments, the preset stimulation patterns are updated by the user. In some embodiments, the present patterns are adjusted via algorithms. In some embodiments, the algorithms can be turned on/off as desired. Since each user's nerve anatomy will be different, the specific nerve branches (e.g., radial, median, and ulnar) activated by the stimulation channels may vary between users. In some embodiments, changing activation/deactivation configurations of common electrodes 102(b), 102(d) can be combined with changing the amplitude of the stimulation signal. In some embodiments, changing the amplitude of the stimulation signal can produce even greater variability in nerve activation. In some embodiments, the amplitude of the stimulation signal is periodically adjusted.

In some embodiments, the controller 200 stimulates the median 302 and radial 300 nerves. For example, during a therapy session, the controller 200 can have a set value for the amplitude of the stimulation signal. In some embodiments, the set value is maintained for stimulating each nerve 300, 302. In certain other embodiments, the controller 200 has a set value that is different for each nerve 300, 302. In some embodiments, the user can adjust the one or more set values during or between therapy sessions. In this way, the user can set independent amplitude configurations for each nerve 300, 302. In some embodiments, the stimulation amplitude used to activate different nerves 300, 302 can be adjusted independently. In some embodiments, the user can set the stimulation amplitude for each nerve based on a minimum activation level. In some embodiments, the minimum activation level can be determined during calibration of the device 100.

In some embodiments, the controller 200 automatically adjusts the stimulation amplitude of the nerves 300, 302 over time. In some embodiments, tremor features can be extracted from the sensors 112 at the wrist to provide characteristic information about tremor phenotypes that may be leveraged by the controller 200 to automatically adjusts the stimulation amplitude of the nerves 300, 302 over time. These adjustments can further improve diagnosis, prognosis, and/or therapeutic outcomes. Kinematic measures can correlate with tremor severity. The machine learning algorithms incorporated in the device 100 and controller 200 as disclosed, for example, herein can automatically adjusts the stimulation amplitude applied by the device 100 and controller 200.

The rule generation engine 206 can employ machine learning modeling along with signal processing techniques to determine rules, where machine learning modeling and signal processing techniques include but are not limited to: supervised and unsupervised algorithms for regression and classification. Specific classes of algorithms include, for example, Artificial Neural Networks (Perceptron, Back-Propagation, Convolutional Neural Networks, Recurrent Neural networks, Long Short-Term Memory Networks, Deep Belief Networks), Bayesian (Naive Bayes, Multinomial Bayes and Bayesian Networks), clustering (k-means, Expectation Maximization and Hierarchical Clustering), ensemble methods (Classification and Regression Tree variants and Boosting), instance-based (k-Nearest Neighbor, Self-Organizing Maps and Support Vector Machines), regularization (Elastic Net, Ridge Regression and Least Absolute Shrinkage Selection Operator), and dimensionality reduction (Principal Component Analysis variants, Multidimensional Scaling, Discriminant Analysis variants and Factor Analysis). In some embodiments, the controller 200 can use the rules to automatically determine outcomes. The controller 200 can also use the rules to control or change settings of the device 100, including but not limited to stimulation parameters (e.g., stimulation amplitude, frequency, patterned (e.g., burst stimulation), intervals, time of day, individual session or cumulative on time, and the like).

In this way, in some embodiments, the controller 200 can employ machine learning algorithms.

For example, in some embodiments, the controller 200 employs a machine learning algorithm to set-up and/or automatically adjust the stimulation amplitude(s) applied by the device 100. The adjustments can occur over time for specific peripheral nerves. In some embodiments, the machine learning algorithm determines a relative percentage of amplitude across all affected nerves 300, 302. In some embodiments, the relative percentages of amplitude can be considered offsets or factors. In some embodiments, the user only sets the amplitude of the stimulation signal for one of the nerve targets. In some embodiments, the controller 200 sets the amplitudes for the remaining nerve(s) based on, for example, machine learning. For example, if the median nerve 302 is measured or set to an amplitude of x, the controller 200 can set the radial nerve 300 to an amplitude using a factor or offset of ¾ times the amplitude x. In some embodiments, the controller 200 further can set the ulnar nerve to an amplitude using a factor or offset of ⅛ times the amplitude x. As explained, these factors or offsets (e.g., ¾ and ⅛) are only exemplary and can have any value.

In some embodiments, training of the machine learning algorithm results in a determinization of typical offsets or factors for users. In some embodiments, the learning algorithm is trained by anatomical stimulation studies. In some embodiments, the user can further personalize the initial factors or offsets determined by the machine learning algorithm.

In some embodiments, the initial factors or offsets are selected by the user. In some embodiments, the user creates the initial offsets or factors during set up of the device 100. In some embodiments, the machine learning algorithm personalizes the initial offsets or factors. In some embodiments, the machine learning algorithm is trained by, for example, anatomical stimulation studies to personalize the initial factors or offsets. In some embodiments, the machine learning algorithm is trained by evaluating the signals collected by the signal collection engine 202 to personalize the initial factors or offsets.

In some embodiments, a range of proportionality of amplitudes for the stimulation signals can be approximately 50-200%. In other embodiments, the range of proportionality of amplitudes for the stimulation signals can have a different range. The range endpoints are not limited and can have any value.

In some embodiments, the machine learning algorithm employed by the controller 200 for amplitude set-up and/or automatic adjustment can be trained and learn by evaluating anatomical stimulation studies. In some embodiments, inputs to the machine learning algorithm can include the signals collected by the signal collection engine 202. The signals collected by the signal collection engine 202 can come from the sensors 112. Sensors 112 can include, for example, biomechanical sensors configured to, for example, measure motion, and/or bioelectrical sensors (e.g., EMG, EEG, and/or nerve conduction sensors). Sensors 112 can include, for example, cardiac activity sensors (e.g., ECG, PPG), skin conductance sensors (e.g., galvanic skin response, electrodermal activity), motion sensors (e.g., accelerometers, gyroscopes), force transducers, and an inertial measurement unit (IMU). In some embodiments, the force transducer is configured to measure tightness of the band 174 on the wrist. In some embodiments, the sensor 112 is configured to measures the wrist circumference of the user. The sensors 112 can be in the band 174 or device 100.

Other inputs to the machine learning algorithm can include demographics (age, sex, wrist size, body-mass index, race, ethnicity, etc.), medical history (ET medications, years since initially ET diagnosis, etc.), and/or tremor characteristics (tremor frequency, tremor amplitude, etc.).

In some embodiments, the sensor 112 measures the tightness of the band 174 on the wrist and/or wrist circumference of the user. In some embodiments, the sensor 112 can measure values during set-up of the device 100, after set-up, before, during, and/or after a therapy session. In some embodiments, the sensor 112 can measure values in real time.

In some embodiments, the measurements (e.g., tightness and/or circumference) by the sensor 112 are used by the machine learning algorithm to set-up and/or automatically adjust the stimulation amplitude(s) applied by the device 100. For example, in some embodiments, the controller 200 initially sets and automatically adjusts the stimulation amplitude throughout therapy based on a combination of the tightness and/or circumference measurements provided by the sensors 112 and machine learning. In some embodiments, the controller 200 initially sets and automatically adjusts the stimulation amplitude without active intervention by the user.

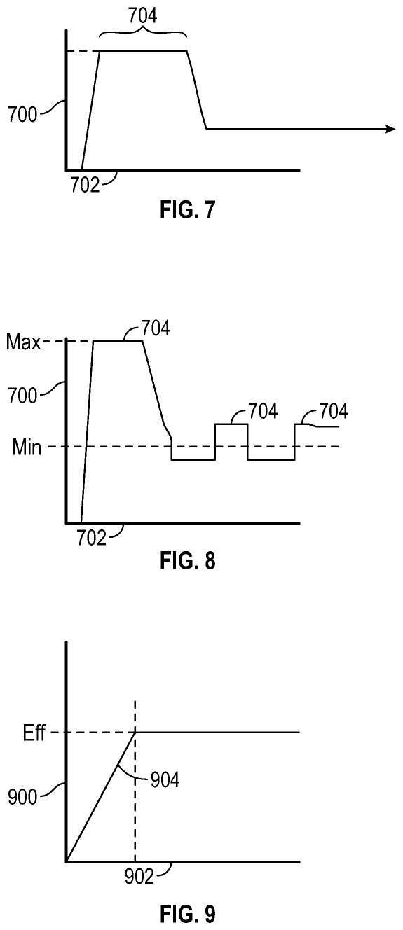

In some embodiments, the controller 200 performs adjustments to the parameters of the waveform applied during therapy. In some embodiments, these adjustments are made to the stimulation amplitude. Some adjustments to the stimulation amplitude during therapy are illustrated in FIGS. 7 through 9. For example, FIG. 7 is a graph of stimulation amplitude 700 v. time 702 showing a temporary, high stimulation amplitude or kick-start 704 being applied to promote the therapeutic response of the user during a therapy session. FIG. 8 is a graph of stimulation amplitude 700 v. time 702 showing a periodic, high stimulation amplitude or kick-start 704 being applied to promote the therapeutic response of the user during a therapy session. FIG. 9 is a graph of kinematic tremor measurements 900 v. time 902 taken during treatment and used to determine whether a change in stimulation amplitude, such as the kick-start 704, will promote the therapeutic response of the user. In some embodiments, the slope 904 of the tremor-session time data can be used to determine whether the controller 200 applies the kick-start 704.

In some embodiments, the controller 200 can include one or more settings (e.g., preset values and/or initial settings) to determine, for example, the stimulation amplitude for the kick-start 704. In some embodiments, the one or more setting can include a preset amplitude setting for the median nerve, a preset amplitude setting for the radial nerve, an initial sensation setting for the median and/or radial nerve, and/or a maximum tolerable sensation setting for the median and/or radial nerve. In some embodiments, these settings are adjusted or entered during a calibration and/or preset stage of setting up the device 100. In some embodiments, these settings are adjusted or entered after the calibration and/or preset stage.

Referring to FIG. 7, in some embodiments, the kick-start 704 is applied by the controller 200. In some embodiments, the kick-start 704 is implemented for a period of time, for example, 10, 20, 30, 60, 90, 120 seconds, or 1, 2, 3, 5, 10, 15, 20, 30 minutes, or more or less, or ranges incorporating any two of the foregoing values. For example, the user can initiate the kick-start 704 to raise the stimulation amplitude to a higher value for a period of time. In some embodiments, the period of time can be predetermined. In some embodiments, the kick-start 704 can promote the user's therapeutic response. In some embodiments, after the kick-start 704, the stimulation amplitude can be lowered.

In some embodiments, the user can select an initial first sensation amplitude preset and/or a maximum tolerable amplitude preset. In some embodiments, the initial first sensation amplitude preset and/or the maximum tolerable amplitude preset are selected during initial calibration of the device 100. In some embodiments, the controller 200 can use the maximum tolerable amplitude preset as the initial amplitude for the therapy for a short period of time followed by a reduction of the amplitude for the remainder of the therapy.

Referring to FIG. 8, in some embodiments, the kick-start 704 is periodically applied by the controller 200. In some embodiments, the kick-start 704 can be initiated by the user. For example, the user can initiate the kick-start 704 to raise the stimulation amplitude to a higher value for a period of time. In some embodiments, the period of time can be predetermined. In some embodiments, the kick-start 704 can promote the user's therapeutic response. In some embodiments, the kick-start 704 is leveraged over the course of the therapy. For example, in some embodiments, the controller 200 delivers the kick-start 704 multiple times throughout the therapy session. In some embodiments, the amplitude of the kick-start 704 is determined based on the maximum tolerable amplitude preset. In some embodiments, the amplitude of the kick-start 704 varies during the therapy session. In some embodiments, by periodically applying the kick-start 704, the potential for habituation by the user can be reduced.

Referring to FIG. 9, in some embodiments, the slope 904 of the tremor-session time data can be used to determine whether the controller 200 applies the kick-start 704. For example, in some embodiments, the controller 200 adapts the stimulation amplitude if the slope 904 of the tremor-session time data exceeds a predetermined slope. In some embodiments, if the predetermined slope is exceeded, the controller 200 can apply the kick-start 704 as illustrated in FIGS. 7 and/or 8. In other embodiments, parameters other than or in addition to the stimulation amplitude of the waveform are changed by the controller 200.

In some embodiments, the stimulation amplitude required to elicit paresthesia sensations during therapy is dependent in part on the relative conductance between the electrodes 102 and the skin of the user. For example, if the electrode 102 is making full contact with the skin more current may penetrate the skin layer and activate the targeted nerves 300, 302. In some embodiments, a conductive medium (e.g., water, sweat, electrode gel) located between the electrode 102 and the skin can further promote the current to penetrate the skin layer and activate the targeted nerves 300, 302. In some embodiments, reducing a system impedance between the electrode(s) 102 and the skin can lower the level of the stimulation amplitude necessary for paresthesia sensations during therapy.

In some embodiments, the controller 200 automatically adjusts the stimulation amplitude based on the system impedance measured by the sensor 112. The sensor 112 can include, for example, skin conductance sensors (e.g., galvanic skin response, electrodermal activity). The sensor 112 can be in the band 174 or device 100.

In some embodiments, the sensor 112 measures the system impedance at periodic intervals. In some embodiments, the sensor 112 measures the system impedance when triggered by the controller 200. In some embodiments, the sensor 112 measures the system impedance in real-time. In some embodiments, the controller 200 adjusts the amplitude stimulation based on the system impedance to maintain a consistent level of paresthesia sensations over time. In some embodiments, the user can set an initial stimulation amplitude. The controller 200 can automatically increase or decrease the stimulation amplitude based on the measured system impedance. In some embodiments, adjusting stimulation amplitude can maintain a consistent level of paresthesia sensations over time despite changes in the electrode-skin contact.

In some embodiments, the controller 200 adjusts the amplitude stimulation based on an orientation of the device 100. In some embodiments, the system impedance may vary as the user changes the orientation of the device 100 by, for example, raising or lowering the device 100 relative to their body, e.g., torso. For example, as the user raises and rotates the device 100, the system impendence may change due to, for example, a change in contact between the device 100 (e.g., electrode(s) 102) and the skin of the user. Similarly, as the user lowers and rotates the device 100 from above to below their waist, the system impendence may change due to, for example, a change in contact between the device 100 (e.g., electrode(s) 102) and the skin of the user. In some embodiments, a location(s) of the electrodes 102 about the circumference of the band 174 may contribute to variations in system impedance as the user changes the orientation of the device 100. In some embodiments, wrist motion is determined by the sensor 112 (e.g., inertial measurement units (IMUs). For example, in some embodiments, the sensor 112 provides position and/or movement data indicative of the orientation of the device 100 to the controller 200. In some embodiments, the controller 200 may leverage the data to adjust the amplitude of the stimulation signal from the device 100 to increase the efficacy of the treatment.

In some embodiments, the relationship between stimulation amplitude and system impedance may be different for each user. To customize the relationship for a specific user, the controller 200 can employ machine learning, in some embodiments. In some embodiments, the machine learning can be trained on data collected during calibration and/or one or more therapy sessions. For example, the data can be collected from the user-selected amplitudes and measured system impedance during one or more therapy sessions. In some embodiments, the machine learning algorithm can establish the relationship between stimulation amplitude and system impedance for the user based on the collected data. In some embodiments, the controller 200 uses regression analysis to establish the relationship between stimulation amplitude and system impedance for the user based on the collected data.

In some embodiments, the controller 200 can apply the relationship in future therapy sessions to automatically adjust the stimulation amplitude. In other embodiments, the controller 200 monitors the change in impedance over time. In such an embodiment, the control 200 can employ the initial impedance/amplitude relationship as a starting point for determining the stimulation amplitude and then can adjust the amplitude stimulation if there is a significant change in system impedance measured by the sensor 112.

In some embodiments, the sensor 112 can monitor the user's response to the changes in the system impedance and provide certain measurements to the controller 200. The controller 200, based on the measurements, can increased or decrease the stimulation amplitude to mimic the user's likely behavior based at least in part on the measured impedance change.

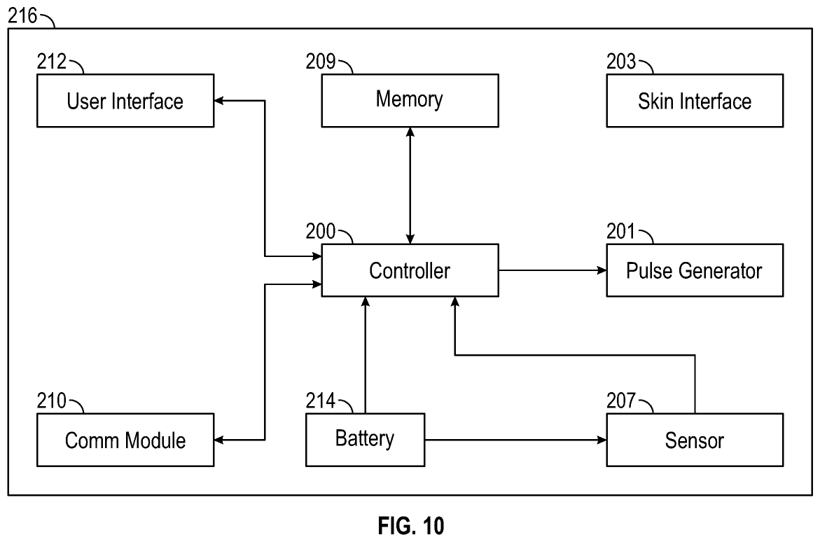

FIG. 10 illustrates a block diagram of another embodiment of a device and system 216 that provides peripheral nerve stimulation. In certain embodiments, the device and system 216 senses a biological measure, a kinematic measure, and/or user satisfaction data. In certain embodiments, the device and system 216 use the biological measure, the kinematic measure, and/or the user satisfaction data to customize or modify the delivery of an electrical stimulus.

In some embodiments, the system 216 comprises a pulse generator 201. In certain embodiments, the pulse generator 201 delivers electrical stimulation to a nerve through one or more skin interfaces 203. In certain embodiments, the one or more skin interfaces 203 can be an electrode 102 as described elsewhere herein. In certain embodiments, the one or more skin interfaces 203 sit adjacent to one or more target peripheral nerves. A controller 200 receive one on more signals generated by one or more sensors 207 to control timing and parameters of stimulation. The controller 200 can be the same as or similar to the controller 200 described with respect to FIG. 2 with more or less functionality. In certain embodiments, the controller 200 uses instructions stored in the memory 209 to coordinate receiving signals from the one or more sensors 207. In certain embodiments, the controller 200 uses the received signal to control stimulation delivered by the pulse generator 201. The memory 209 in the system 216 can store signal data from the sensors 207.

In certain embodiments, the system 216 has a communication module 210 to transmit data to other devices or a remote server via standard wired or wireless communication protocols. In certain embodiments, the system 216 is powered by a battery 214. In certain embodiments, the system 216 has a user interface 212. In certain embodiments, the user interface 212 allows the user to receive feedback from the system 216. In certain embodiments, the user interface 212 allows the user to provide input to the system 216 via, e.g., one or more buttons. In certain embodiments, the user provides satisfaction data via the user interface 212. For example, the user can provide input to the user interface 212 in the form of a patient session impression of improvement (PSII) score and/or a patient satisfaction scope. In certain embodiments, the user interface 212 allows a user to receive instructions, feedback, and control aspects of the delivered stimulation, such as intensity of the stimulation.

In certain embodiments, the controller 200 can receive kinematic and/or satisfaction data to determine a method for varying multiple stimulation parameters based on adaptive learning as disclosed herein. In certain embodiments, the controller 200 causes the device 100 to adjust one or more parameters of a first electrical stimulus based at least in part on the kinematic and/or satisfaction data.

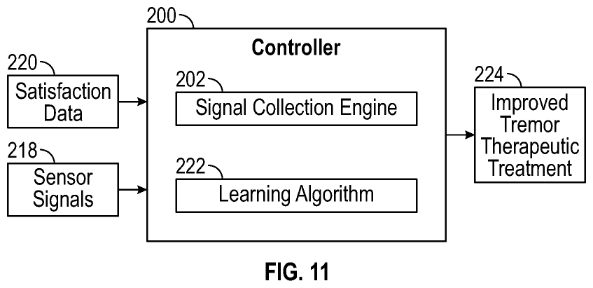

FIG. 11 illustrates a block diagram of an embodiment of a controller 200 that can be implemented with the hardware components described with respect to FIGS. 1A, 1B, 1C, and 10. The controller 200 can include multiple engines for performing the processes and functions described herein. The engines can include programmed instructions for performing processes as discussed herein for detection of input conditions, processing data, and control of output conditions. The engines can be executed by the one or more hardware processors of the neuromodulation (e.g., neurostimulation) device 100 alone or in combination with the base station 150, the user interface device 150, and/or the cloud. The programming instructions can be stored in the memory 209. The programming instructions can be implemented in C, C++, JAVA, or any other suitable programming languages. In some embodiments, some or all of the portions of the controller 200 including the engines can be implemented in application specific circuitry such as ASICs and FPGAs. Some aspects of the functionality of the controller 200 can be executed remotely on a server (not shown) over a network. While shown as separate engines, the functionality of the engines as discussed below is not necessarily required to be separated. Accordingly, the controller 200 can be implemented with the hardware components described above with respect to FIGS. 1A, 1B, 1C, and 10.

The controller 200 can include a signal collection engine 216. The signal collection engine 216 can enable acquisition of raw/sensor data 218 from the sensors 112, 207 embedded in the device 100 as well as user satisfaction data 220. The sensor data 218 can include but is not limited to accelerometer or gyroscope data from the sensors 112, 207 (e.g., IMU). In certain embodiments, the sensor data 218 can include test kinematic data taken during a therapy session. In certain embodiments, the sensor data 218 can include passive kinematic data. Passive kinematic data is data collected at times outside of the therapy session.

In some embodiments, the neuromodulation, e.g., neurostimulation device 100 or the user interface device 150 with sensors can collect kinematic or motion data (test and/or passive data), or data from other sensors, can measure data over a longer period of time, for example 1, 2, 3, 4, 5, 10, 20, 30 weeks, 1, 2, 3, 6, 9, 12 months, or 1, 2, 3, 5, 10 years or more or less, or ranges incorporating any two of the foregoing values, to determine features, or biomarkers, associated with the onset of tremor diseases, such as essential tremor, Parkinson's disease, dystonia, multiple sclerosis, Lyme disease, etc. Biomarkers could include specific changes in one or more features of the data over time, or one or more features crossing a predetermined threshold. In some embodiments, features of tremor inducing tasks have been stored on the neurostimulation device 100 and used to automatically activate sensors when those tremor inducing tasks are being performed, to measure and store data to memory during relevant times.

The devices, systems and methods described herein are used to treat Lyme disease (e.g., its associated symptoms) in some embodiments. The inflammation associated with Lyme disease is reduced in one embodiment (including for example, long term or chronic inflammation and/or flare ups). Resulting neurological conditions are treated in some embodiments, including but not limited to, weakness, numbness, nerve damage, and facial muscle paralysis. In addition to Lyme disease, chronic fatigue syndrome and its associate symptoms, such chronic inflammation, flare ups etc. are treated according to several embodiments. Treatment may be accomplished by, for example, vagal stimulation and/or sympathetic/parasympathetic balance. Systems and methods to reduce habituation and/or tolerance to nerve stimulation (such as vagus nerve stimulation via an earpiece) are provided in several embodiments by, for example, introducing variability in stimulation parameter(s), as described herein.

The satisfaction data 220 can include but is not limited to subjective data provided by the user. The subjective data can relate to pre or post treatment and/or patient activities of daily living (ADL). In certain embodiments, the user inputs a value that reflects a level of satisfaction. The level of satisfaction can be selected from a predetermined range. In certain embodiments, the range is from 1 to 4. Of course, the range can be any range and is not limited to 1 to 4. For example, the user can provide input to the user interface 212 in the form of a patient session impression of improvement (PSII) score and/or a user satisfaction score.

In some embodiments, the signal collection engine 216 can also perform signal preprocessing on the raw data. Signal preprocessing can include noise filtering, smoothing, averaging, and other signal preprocessing techniques to clean the raw data. In some embodiments, portions of the signals can be discarded by the signal collection engine 216. In some embodiments, portions of the signals are associated with a time stamp or other temporal indicator.

In certain embodiments, the controller 200 determines a level of patient therapeutic benefit based on the passive kinematic data from the sensor signals 218 without requiring the user to input a subjective satisfaction level. In certain embodiments, the controller 200 collects sensor signals 218 in the form of kinematic data measured during the therapy session along with satisfaction data 220 input by the user. In this way in certain embodiments, the controller 200 can determine a level of patient therapeutic benefit based on both the passive kinematic data and the patient provided subjective satisfaction level.

The controller 200 can further include a learning algorithm 222. In certain embodiments, the learning algorithm 222 selects from methods for varying parameter(s) employed during therapy session based on adaptive learning to improve tremor therapeutic treatment 224.

In certain embodiments, the learning algorithm 222 can select from a plurality of stimulation parameters (e.g., burst frequency and pulse frequency) to vary one parameter across one or more nerves (e.g., median and/or radial nerve) and/or select multiple stimulation parameters to vary across one or more nerves.

In certain embodiments, the plurality of stimulation parameters accessed by the learning algorithm 222 can be a subset of all of the stimulation parameters and or patterns of applying stimulation parameters. For example, in certain embodiments, the learning algorithm 222 selects the response profile(s) for which a positive outcome is predicted by the learning algorithm 222. In certain embodiments, the learning algorithm 222 modifies the one or more parameters of the selected stimulation parameters based on the individual user to further personalize the stimulation parameters and improve neurostimulation therapy outcomes.

The learning algorithm 222 can automatically determine a correlation between the satisfaction data 220 and/or the sensor signals 218 and neurostimulation therapy outcomes. Outcomes can include, for example, identifying patients who will respond to the therapy (e.g., during an initial trial fitting or calibration process) based on tremor features of kinematic data from the sensor signals 218 (e.g., approximate entropy), predicting stimulation settings for a given patient (based on their tremor features) that will result in the best therapeutic effect (e.g., dose, where parameters of the dose or dosing of treatment include but are not limited to duration of stimulation, frequency and/or amplitude of the stimulation waveform, and time of day stimulation is applied), predicting patient tremor severity at a given point, predicting patient response over time, examining patient medication responsiveness combined with tremor severity over time, predicting response to transcutaneous or percutaneous stimulation, or implantable deep brain stimulation or thalamotomy based off of tremor features and severity over time, and predicting ideal time for a patient to receive transcutaneous or percutaneous stimulation, or deep brain stimulation or thalamotomy based off of tremor features and severity over time, predicting patient reported therapy outcomes or patient reported satisfaction using tremor features assessed kinematic measurements from the device; predicting patient response to undesirable user experience using tremor features assessed from kinematic measurements and patient usage logs from the device where undesirable user experiences can include but are not limited to device malfunctions and adverse events such as skin irritation or burn; predict patient response trends based on tremor severity where trends can be assessed across total number of sessions, within an individual patient, or across a population of patients; predicting or classifying subtypes of tremor to predicting patient response based on kinematic analysis of tremor features; predicting or classifying subtypes of tremor to provide guidance for individually optimized therapy parameters; predicting or classifying subtypes of tremor to optimize the future study design based on subtypes (e.g., selecting specific subtypes of essential tremor for a clinical study with specific design addressing therapy need for the subtype); and predict patient or customer satisfaction (e.g., net promoter score) based on patient response or other kinematic features from measure tremor motion.

In some embodiments, the neuromodulation, e.g., neurostimulation device 100 or the user interface device 150 with sensors 218 can collect kinematic or motion data, or data from other sensors, when a tremor inducing task is being performed. The user can be directly instructed to perform the task, for example via the display 116 on the device 100 or audio. In some embodiments, features of tremor inducing tasks are stored on the device 100 and used to automatically activate sensors to measure and store data to memory during relevant tremor tasks. The period of time for measuring and storing data can be, for example, 1-180 seconds such as 10, 20, 30, 60, 90, 120 seconds, or 1-60 minutes such as 1, 2, 3, 5, 10, 15, 20, 30 minutes, or 1-12 hours such as 1, 2, 3, 4, 5, 6, 7, 8 hours or more or less, or ranges incorporating any two of the foregoing values. Based on a training set of data from a cohort of previous wearers with tremor or another condition, the learning algorithm 222 can detect features that are correlated with response to stimulation such that the patient or physician can be presented with one or more response profiles. The one or more response profiles can correspond to neurostimulation therapy that has a qualitative likelihood for patient response.

In another embodiment, features can be correlated with the type of tremor measured, such as essential tremor, resting tremor (associated with Parkinson's Disease), postural tremor, action tremor, intention tremor, rhythmic tremor (e.g., a single dominant frequency) or mixed tremor (e.g., multiple frequencies). In some embodiments, essential tremor pathology can include, for example, a primarily cerebellar variant with Bergmann gliosis and Purkinje cell torpedoes, and a Lewy body variant, and a dystonic variant, and a multiple sclerosis variant, and a Parkinson disease variant. The type of tremor most likely detected can be presented to the patient or physician as a diagnosis or informative assessment prior to receiving stimulation or to assess appropriateness of prescribing a neuromodulation, e.g., stimulation treatment. In another embodiment, various response profiles may be applied based on the tremor type determined; different profiles could include changes in stimulation parameters, such as frequency, pulse width, amplitude, burst frequency, duration of stimulation, or time of day stimulation is applied. In one embodiment, the user interface device 150 can include an app that asks the patient to take a self-photograph or self-video, which has the patient perform a task that has both posture and intention actions.

In some embodiments, the neuromodulation, e.g., neurostimulation device 100 can apply transcutaneous stimulation to a patient with tremor that is a candidate for implantable deep brain stimulation or thalamotomy. Tremor features and other sensor measurements of tremor severity will be used to assess response over a prespecified usage period, which could be 1 month or 3 months, or 5, 7, 14, 30, 60, or 90 days or more or less. The response to transcutaneous stimulation as assessed, for example, by the learning algorithm 222 described herein using sensor measurements from the device and/or patient satisfaction data can advantageously provide an assessment of the patient's likelihood to respond to implantable deep brain stimulation or other implantable or non-implantable therapies.

In some embodiments, the learning algorithm 222 develops rules between the satisfaction data 220 and/or sensor signals 218 and one or more parameters of one or more response profiles that correspond to neurostimulation therapy outcomes. The learning algorithm 222 can employ machine learning modeling along with signal processing techniques to determine rules, where machine learning modeling and signal processing techniques include but are not limited to: supervised and unsupervised algorithms for regression and classification. Specific classes of algorithms include, for example, Artificial Neural Networks (Perceptron, Back-Propagation, Convolutional Neural Networks, Recurrent Neural networks, Long Short-Term Memory Networks, Deep Belief Networks), Bayesian (Naive Bayes, Multinomial Bayes and Bayesian Networks), clustering (k-means, Expectation Maximization and Hierarchical Clustering), ensemble methods (Classification and Regression Tree variants and Boosting), instance-based (k-Nearest Neighbor, Self-Organizing Maps and Support Vector Machines), regularization (Elastic Net, Ridge Regression and Least Absolute Shrinkage Selection Operator), and dimensionality reduction (Principal Component Analysis variants, Multidimensional Scaling, Discriminant Analysis variants and Factor Analysis). In some embodiments, the controller 200 can use the rules developed between features and one or more parameters to automatically determine response profiles that correspond to neurostimulation therapy outcomes. The controller 200 can also use the one or more response profiles to control or change settings of the neurostimulation device, including but not limited to stimulation parameters (e.g., stimulation amplitude, frequency, patterned (e.g., burst stimulation), intervals, time of day, individual session or cumulative on time, and the like).

Accordingly, the one or more response profiles that correspond to neurostimulation therapy can improve operation of the neuromodulation, e.g., neurostimulation device, and advantageously and accurately identify potential candidates for therapy and well as various disease state and therapy parameters over time. The generated one or more response profiles that correspond to neurostimulation therapy can be saved in the memory 110 and/or memory 209. For example, the methods for varying one or more stimulation parameters can be generated and stored prior to operation of the neurostimulation device 100. Accordingly, in some embodiments, the controller 200 can apply the saved one or more profiles based on new data collected by the sensors 112, 207 to determine outcomes or control the neuromodulation, e.g., neurostimulation device 100.

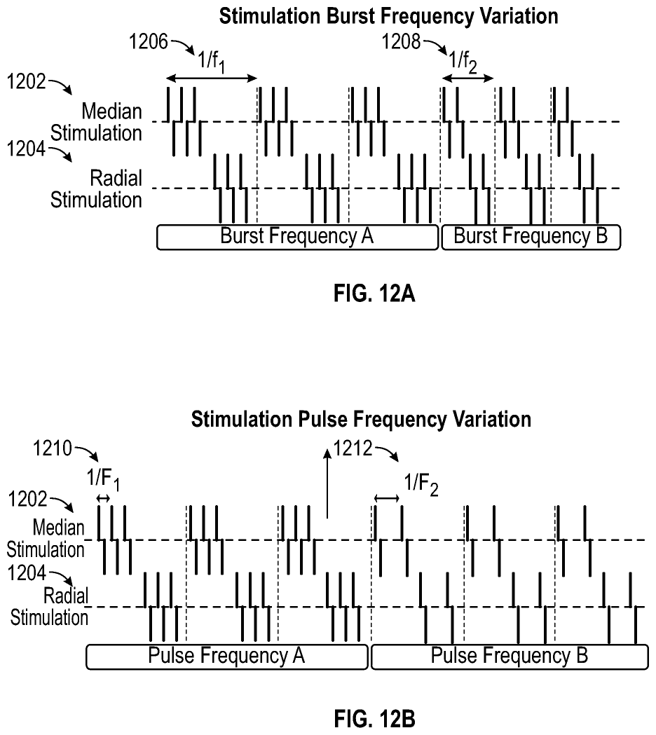

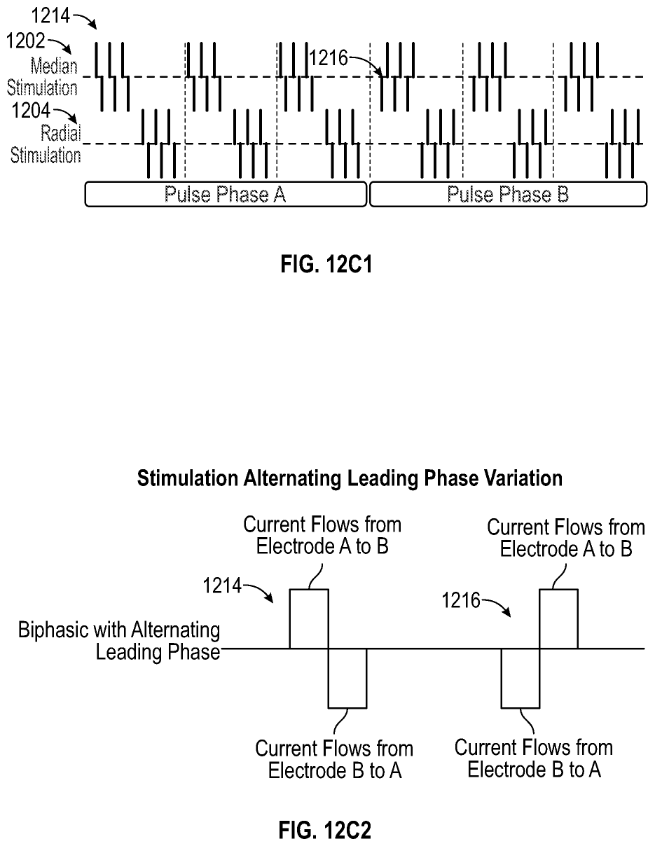

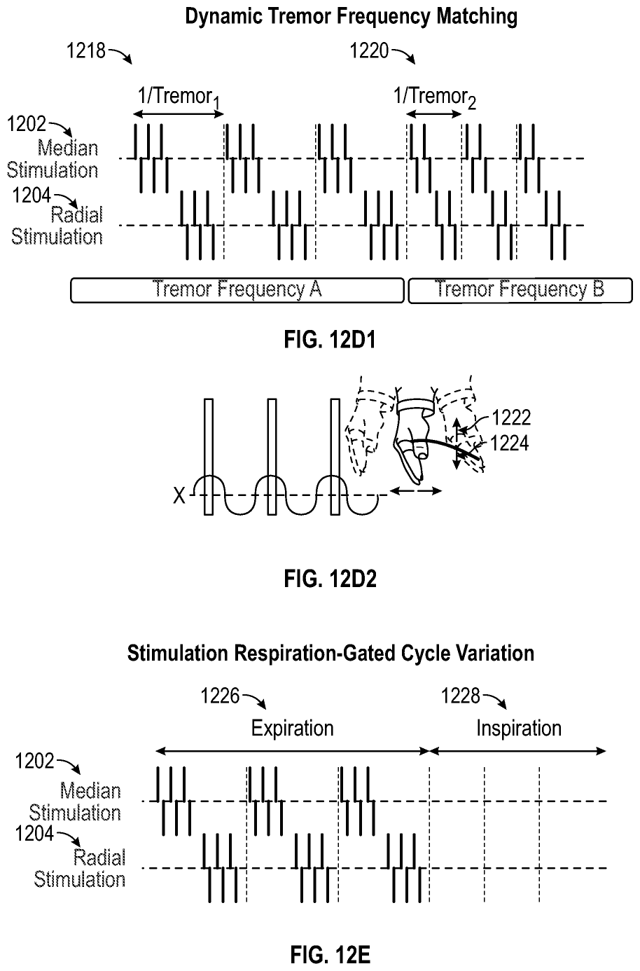

FIGS. 12A-C2 illustrate examples of how stimulation parameters (e.g., burst frequency, pulse frequency, and pulse phase) are varied between two or more prespecified values as stimulation is alternated across two nerves (e.g., median and radial nerve). The plots show patterns of current delivered by the device 100 over time.

FIG. 12A illustrates an embodiment of the device 100 that delivers patterned stimulation to the median nerve 1202 and radial nerve 1204 where burst frequency is varied after a prespecified time period or prespecified number of bursts. As is illustrated in FIG. 12A, the burst frequency is initially burst frequency A with a period of 1/f₁ 1206. The burst frequency subsequently changes to burst frequency B with a different period of 1/f₂ 1208. FIG. 12A is only exemplary and is not intended to limit the variations in burst frequency to the illustrated values or the number of different burst frequencies. Further, while FIG. 12A illustrates the variation occurring across multiple nerves (e.g., median and radial nerves), the disclosure is not so limited. The disclosed variations can be applied to only a single nerve.

In some embodiments, burst frequency variability is centered on an about, at least about, or no more than about 0.1, 0.2, 0.25, 0.3, 0.4, 0.5, 1, 1.5, 2, 2.5, 3, 3.5, 4, 4.5, 5, 5.5, or 6 Hz or more or less window (or ranges including any two of the foregoing values), or any combination thereof, around a calibration frequency measured from a tremor-inducing task, such as a postural hold. In certain embodiments, if the measured tremor frequency is at a lower edge of a partial tremor frequency range (e.g., a 3-12 Hz window), the burst frequency variation window would not go below 3 Hz. In certain embodiments, if the measured tremor frequency is at the higher edge of a partial tremor frequency range (e.g., a 3-12 Hz window), the burst frequency variation window would not go above 12 Hz. In an alternative embodiment, burst frequency variability is applied within the full or partial tremor frequency range, for example between 3-12 Hz for essential tremor. This alternative embodiment may have the advantage of not requiring the user to perform a tremor inducing task for calibration. In yet another embodiment, the range of values for burst frequency variability is set based on the minimum and maximum tremor frequencies measured from multiple tremor-inducing task measurements. In some embodiments, burst frequency variability can avoid exact alignment to the pathological oscillation frequency over time and enhance the therapeutic response compared to a constant burst frequency. In some embodiments, the rate of change of the burst frequency parameter may be between 0.001 Hz/s (i.e., slowest rate of change of burst frequency being in increments of 0.1 Hz every 100 sec) to 100 Hz/s (i.e., fastest rate of change of burst frequency being in increments of 8 Hz burst frequency change every tremor cycle and rounding up).

FIG. 12B illustrates an embodiment of the device 100 that delivers patterned stimulation to the median nerve 1202 and radial nerve 1204 where pulse frequency is varied after a prespecified time period or prespecified number of bursts. As is illustrated in FIG. 12B, the pulse frequency is initially pulse frequency A with a period of 1/F₁ 1210. The pulse frequency subsequently changes to pulse frequency B with a different period of 1/F₂ 1212. FIG. 1B is only exemplary and is not intended to limit the variations in pulse frequency to the illustrated values or number of pulse frequencies. Further, while FIG. 12B illustrates the variation occurring across multiple nerves (e.g., median and radial nerves), the disclosure is not so limited. The disclosed variations can be applied to only a single nerve.

In some embodiments, the pulse frequency of electrical stimulation applied to a peripheral nerve or neuron can govern how frequently the stimulated nerve or neuron generates an action potential. In some cases, peripheral nerve fibers can be activated to generate an action potential with every stimulation pulse at pulse frequencies of less than approximately 1,000 Hz, if the stimulation pulse width and amplitude are sufficiently high. In some cases, stimulation of the median nerve with pulse frequencies of 5, 50, 100, 150, and 200 Hz can evoke a response of the VIM thalamus, as measured with implanted microelectrodes during a surgical procedure. Moreover, the pulse frequency that generates the maximal amplitude evoked response of the VIM thalamus can vary across subjects. In some embodiments, pulse frequency is varied between 5-200, 5-150, 5-100, 5-50, 50-200, 50-150, 50-100, 100-200, 100-150, or 150-200 Hz (or ranges including any two of the foregoing values), which can enhance therapeutic response compared to a constant pulse frequency. Changes in pulse frequency may be implemented by changing the timing of pulse delivery directly, or by keeping the timing fixed and alternating stimulation amplitude on a pulse-to-pulse basis to change the effective pulse frequency. For example, setting every 1 of 2 pulses to a low stimulation amplitude, which is subthreshold for recruitment of neurons or nerves, can reduce the effective pulse frequency by ½. In some embodiments, the rate of change of the pulse frequency parameter may be between 0.001-10,000 Hz/s. In some embodiments, varying pulse frequency may generate activity in the brain that modulates pathological cortical dynamics associated with hand tremor. An additional advantage of varying pulse frequency is that this type of stimulation can elicit a more natural paresthesia sensations, similar to tapping, pressure, touch, and/or vibration sensations experienced during daily life.

In one embodiment the pulse frequency may be from about 1 to about 5000 Hz, about 1 Hz to about 500 Hz, about 5 Hz to about 50 Hz, about 50 Hz to about 300 Hz, or about 150 Hz, or other ranges including any two of the foregoing values. In some embodiments, the pulse frequency may be from 1 kHz to 20 KHz.

FIGS. 12C1-C2 illustrate embodiments of the device 100 that deliver biphasic patterned stimulation to the median nerve 1202 and radial nerve 1204 where the leading pulse phase changes or alternates (e.g., one or more pulses or bursts of a cathodic-first phase of current flowing from electrode 1 to 2 followed by one or more pulses or bursts of an anodic-first phase of current flowing from electrode 2 to 1 or vice versa) after (1) a prespecified time period (FIG. 12C1), (b) a prespecified number of bursts (FIG. 12C1), or (c) a prespecified number of pulses (FIG. 12C2). In this way, at least some of the stimulation pulses delivered will have a different leading first phase as opposed to all of the stimulation pulses having a constant cathodic-first phase pattern or a constant anodic first phase pattern (e.g., FIG. 12A).

As is illustrated in FIG. 12C1, the leading pulse phase is pulse phase A 1214 (e.g., a current flows initially during the pulse from electrode 1 to electrode 2) during each pulse for a prespecified time period or number of bursts. In the illustrated embodiment of FIG. 12C1, the leading pulse phase A 1214 is maintained for a series of three bursts with each burst comprising three pulses. The leading pulse phase subsequently alternates to pulse phase B 1216 (e.g., a current flows initially during the pulse from electrode 2 to electrode 1).

In the illustrated embodiment of FIG. 12C1, the leading pulse phase B 1216 is maintained for a series of three bursts with each burst comprising three pulses. FIG. 12C1 is only exemplary and is not intended to limit the variations in leading pulse phase to the illustrated numbers of bursts or pulses. Further, while FIG. 12C1 illustrates the variation occurring across multiple nerves (e.g., median and radial nerves), the disclosure is not so limited. The disclosed variations can be applied to only a single nerve.

As is illustrated in FIG. 12C2, the leading pulse phase is pulse phase A 1214 (e.g., a current flows initially during the pulse from electrode 1 to electrode 2) for one pulse. The leading pulse phase subsequently alternates to pulse phase B 1216 (e.g., a current flows initially during the pulse from electrode 2 to electrode 1) during a second pulse. This alternating pattern can continually repeat at an interval. FIG. 12C2 is only exemplary and is not intended to limit the variations in pulse phase to the illustrated number of pulses or interval for alternating between leading pulse phases. For example, the phase of the leading first pulse can be repeated for two or more pulses before alternating to leading pulse phase B 1216. Further, the leading phase of the second pulse can be repeated for two or more pulses before alternating back to the leading phase of the first pulse. Further, while FIG. 12C2 illustrates the variation occurring across a single nerve, the disclosure is not so limited. The disclosed variations can be applied to multiple nerves (e.g., median and radial nerves).

In some embodiments, prolonged percutaneous stimulation sessions employing a constant pattern of a leading cathodic or anodic first phase may cause electro-chemical changes in the electrode-skin interface even though each pulse is intended to be charge balanced by flowing current in one direction and then reversing the current flow during the pulse (e.g., biphasic). Thus, electro-chemical changes may occur during biphasic operation causing discomfort and adverse biological effects (e.g., skin irritations) due to the movement of charged molecules within the skin caused by the flow of current across the skin. Alternating the leading phase of at least some pulses within the stimulation session such as illustrated in FIGS. 12C1 and 12C2 can mitigate against such adverse biological effects.

FIG. 12D1 illustrates an embodiment of the device 100 that employs dynamic tremor frequency matching. In some embodiments, the device 100 dynamically varies the burst frequency of the patterned stimulation to the median nerve 1202 and/or radial nerve 1204 based at least in part on changes in tremor frequency. In some embodiments, the frequency of the stimulation to the median nerve 1202 and the radial nerve 1204 dynamically tracks real-time, measured changes in the tremor frequency. As is illustrated in FIG. 12D2, in some embodiments, the frequency of the stimulation to a first nerve (e.g., the median nerve 1202) tracks a first phase of the tremor (e.g., hand moving in downward direction 1224) while the stimulation to a second nerve (e.g., the radial nerve 1204) tracks a different phase of the tremor (e.g., hand moving in upward direction 1222).

In some embodiments, the tremor frequency can be between, for example, about 4 Hz and about 12 Hz, between about 3 Hz and about 6 Hz, or about 3 Hz, 4 Hz, 5 Hz, 6 Hz, 7 Hz, 8 Hz, 9 Hz, 10 Hz, 11 Hz, or 12 Hz, or ranges including any two of the foregoing values. In some cases, pathological tremor frequency can change, for example, by more than 2 Hz between tasks and by up to 32% on the same task over time for an individual patient. In some embodiments, phase-locking burst frequency to tremor frequency can improve therapeutic effect.

As is illustrated in FIG. 12D1, the burst frequency for the median and radial stimulation can initially match tremor frequency A with a period of 1/tremor 1218. The tremor frequency can then change to tremor frequency B. The burst frequency can change to tremor frequency B with a different period of 1/tremor 1220 so as to continue matching the frequency of the tremor.

As is illustrated in FIG. 12D2, in some embodiments, the timing of median and radial nerve stimulation can be determined based on the measured, real-time phases of the patient's tremor. For example, median nerve stimulation could be delivered while the tremor is between phases 0-180 degrees (e.g., hand moving in downward direction 324), while radial nerve stimulation could be delivered when the tremor is between phases 180-360 degrees (e.g., hand moving in upward direction 1222). In other embodiments, the radial nerve stimulation can be delivered while the tremor is between phases 0-180 degrees (e.g., hand moving in downward direction 1224), while medial nerve stimulation could be delivered when the tremor is between phases 180-360 degrees (e.g., hand moving in upward direction 1222).

In some embodiments, the durations of the different phases are asymmetrical. For example, in some embodiments, the duration of the first phase (e.g., hand moving in downward direction 1224) is not the same as the duration of the second phase (e.g., hand moving in upward direction 1222). In some embodiments, the device 100 delivers asymmetric stimulation to the first and second nerves based at least in part on the asymmetric phases of the tremor. FIGS. 12D1 and 12D2 are only exemplary and are not intended to limit the variations in the associated timing between nerve stimulation and the real-time phase of the patient's tremor. Further, while FIGS. 12D1 and 12D2 illustrate the variation occurring across multiple nerves (e.g., median and radial nerves), the disclosure is not so limited. The disclosed variations can be applied to only a single nerve.

In some embodiments, the one or more sensors 112, 207 of the device 100 tracks the patient's motion data for the purpose of gauging, real-time, a tremor frequency of the patient and/or phases of the tremor. Once the tremor frequency is observed, the device 100 can use the frequency as a seminal input parameter. The one or more sensors 112, 207 (e.g., inertial measurement unit (IMU), accelerometer, gyroscope, etc.) can measure motion of the patient's extremity for the device 100 to generate motion data; determining tremor frequency from the motion data; and setting the burst frequency to match or closely match (e.g., phase-locked) the measured motion. For example, in some embodiments, an accelerometer configured as the sensor 112, 207 passively measures tremor during a treatment session. In some embodiments, the device 100 continuously tracks the changing tremor characteristics using the one or more sensors 112, 207. In some embodiments, the one or more hardware processor(s) 108, 152 analyze the phase and trigger median 1202 or radial 1204 nerve stimulation accordingly.

In some embodiments, as illustrated in FIG. 12D2, the phase cutoffs (e.g., 0 and 180 degrees) for switching between median 1202 and radial 1204 nerve stimulation can be personalized for the patient. For example, a plurality of different phase cutoffs and ranges could be employed (e.g., 0, 30, 60, 90, 120, 150, 180, 210, 240, 270, 300, 330, and 360 degrees or ranges including any two of the foregoing values) for a series of stimulation sessions. The device 100 could employ the phase cutoffs which produce the best tremor relief. As mentioned above, the phase cutoff can be symmetric or asymmetric depending on, for example, the measured phases of the tremor.

In some embodiments, the dynamic tremor frequency matching burst frequency is centered on about, at least about, or no more than about 0.1, 0.2, 0.25, 0.3, 0.4, 0.5, 1, 1.5, 2, 2.5, 3, 3.5, 4, 4.5, 5, 5.5, or 6 Hz or more or less window (or ranges including any two of the foregoing values), or any combination thereof, around a measured tremor frequency. In certain embodiments, burst frequency variability dynamically matches the pathological oscillation frequency over time. In some embodiments, the rate of change of the measured tremor frequency may be between 0.001 Hz/s (i.e., slowest rate of change of burst frequency being in increments of 0.1 Hz every 100 sec) to 100 Hz/s (i.e., fastest rate of change of burst frequency being in increments of 8 Hz burst frequency change every tremor cycle and rounding up).

FIG. 12E illustrates an embodiment of the device 100 that delivers patterned stimulation to a first nerve (e.g., the median nerve 1202) and a second nerve (e.g., the radial nerve 1204) based at least in part on the patient's respiratory cycle (e.g., respiratory gating). As is illustrated in FIG. 12E, in some embodiments, the timing of median and radial nerve stimulation can be determined based on the measured, real-time phases of the respiratory cycle. Delivering stimulation during a first portion of the respiration cycle and then discontinuing the stimulation during a second portion of the respiration cycle (or vice versa) may enhance autonomic modulatory effects. In some embodiments, a first target nerve can be modulated during an inspiratory phase of the respiratory cycle, and then no stimulation is applied to the first target nerve during an expiratory phase of the respiratory cycle. In this way, the device 100 can be configured to synchronize/gate the stimulation to one or more particular phases of the respiratory cycle.

In some embodiments, stimulation to the median nerve 1202 and the radial nerve 1204 tracks measured changes in the respiratory cycle. As is illustrated in FIG. 12E, in some embodiments, the stimulation to the first and second nerves (e.g., the median nerve 1202 and the radial nerve 1204) occurs during a first portion of the respiration cycle (e.g., expiration 1226) and is discontinued during a second portion of the respiration cycle (e.g., inspiration 1228). In some embodiments, the cycle can then repeat by restimulating the first and second nerves during the next expiration and discontinuing the stimulation during the following inspiration. While the first portion (on stimulation) and second portion (off stimulation) are illustrated as corresponding to the expiration and inspiration phases of the respiratory cycle, the disclosure is not so limited. The first portion and the second portion can correspond to any parts of the respiratory cycle. In some embodiments, the device 100 can analyze a voltage or other signal from the sensor 112, 207 in real-time and can detect different features of the respiratory cycle of the patient. The features detected by the sensor 112, 207 can include, for example, peaks, troughs, and slopes reaching, exceeding, or being less than a predetermined value, for example. Further, in some embodiments, the respiratory cycle can be split into three or more parts (e.g., peaks, troughs, slopes, etc.) based on data received by the sensor 112, 207 with each part corresponding to on or off stimulation.

In some embodiments, the sensor 112, 207 is carried by a respiratory detection device (e.g., a respiration belt) worn by the patient. In some embodiments, the respiratory detection device can further include a communication module, which may be cellular, Bluetooth etc., to communicate with the device 100. In some embodiments, the timing of the stimulation can depend on the algorithm used to trigger the stimulation off of one or more of the measured biological signals (e.g., respiratory cycle) received from the sensor 112, 207. For example, in certain embodiments, the stimulation is triggered based at least in part on whether the burst is a fixed duration, is a percentage of one or more measured biological signals (e.g., respiratory cycle), terminates at a detected phase of a cyclical biological signal (e.g., respiratory cycle), or is based on some other algorithm implemented in the device 100. For example, in some embodiments, the device 100 triggers stimulation when the inspiration and/or expiration phase of the respiratory cycle is detected and continues for at least about, about, or no more than about 5%, 10%, 15%, 20%, 25%, 30%, 35%, 40%, 45%, 50%, 55%, 60%, 65%, 70%, 75%, 80%, 85%, 90%, or 95% of the average measured respiratory interval or ranges including any two of the aforementioned values.

In some embodiments, the device 100 can identify specific points on the respiratory signal that may be more receptive to stimulation. In some embodiments, the stimulation is synchronized to respiratory activity, but the stimulation is not necessarily configured to affect or substantially affect respiratory function (e.g., one or more or respiratory rate, tidal volume, or minute ventilation). For example, dual peripheral nerve stimulation (e.g., medial 1202 and radial 1204 nerve stimulation) can be synchronized to two different phases of the respiratory cycle, to treat the patient without affecting or substantially affecting the patient's respiratory function, and/or affecting or substantially affecting the patient's heart rate or rhythm in some embodiments.

In some embodiments, the stimulation can be synchronized to early expiration, late expiration, early inspiration, and/or late inspiration. The stimulation could also be synchronized to, for example, the 1st⋅2nd⋅3rd⋅4th⋅5th⋅6th⋅7th⋅8th⋅9th, and/or 10th decile chronologically of an inspiratory and/or expiratory cycle, including ranges and/or combinations of any of the foregoing values. In some embodiments the stimulation could be synchronized continuously to the targeted phase(s) of each respiratory cycle, every other respiratory cycle, every third respiratory cycle, on for a predetermined or calculated number of targeted phases(s) of respiratory cycles and off for the same or different predetermined or calculated number of targeted phases(s) of respiratory cycles, or other patterns depending on the desired clinical result. In some embodiments, the stimulation could include a first stimulation mode during a first portion of the respiratory cycle, e.g., expiration, and a second, different stimulation mode during a second portion of the respiratory cycle, e.g., another part of expiration and/or inspiration.

In some embodiments, the durations of the different phases of the respiratory cycle are asymmetrical. For example, in some embodiments, the duration of the first phase (e.g., expiration 1226) is not the same as the duration of the second phase (e.g., inspiration 1228). In some embodiments, the device 100 delivers asymmetric stimulation to the first and second nerves based at least in part on the asymmetric phases of the respiratory cycle. FIG. 12E is only exemplary and is not intended to limit the variations in the associated timing between nerve stimulation and the real-time phase of the patient's respiratory cycle. Further, while FIG. 12E illustrates the variation occurring across multiple nerves (e.g., median and radial nerves), the disclosure is not so limited. The disclosed variations can be applied to only a single nerve.

In some embodiments, the one or more sensors 112, 207 of the device 100 track the patient's motion data for the purpose of gauging, real-time, phases of a respiratory cycle of the patient. Once the respiratory cycle is observed, the device 100 can use the frequency as a seminal input parameter. The one or more sensors 112, 207 can measure respiratory rate and/or content (respiration rate; respiration phase; capnogram; oximetry; spirography), of the patient for the device 100 to generate respiratory data; determining phases of the respiratory cycle from the respiratory data; and turning the stimulation on or off based at least in part on the measured respiration. For example, in some embodiments, the respiratory detection device passively measures respiration during a treatment session. In some embodiments, the device 100 continuously tracks the changing respiration characteristics using the respiratory detection device. In some embodiments, the one or more hardware processor(s) 108, 152 analyze the respiratory cycle and trigger median 1202 or radial 1204 nerve stimulation accordingly.

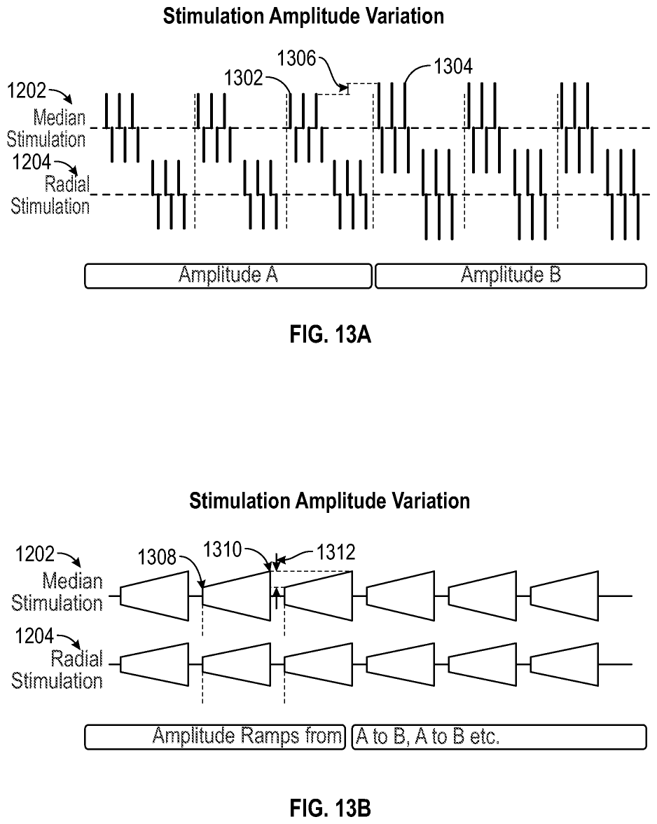

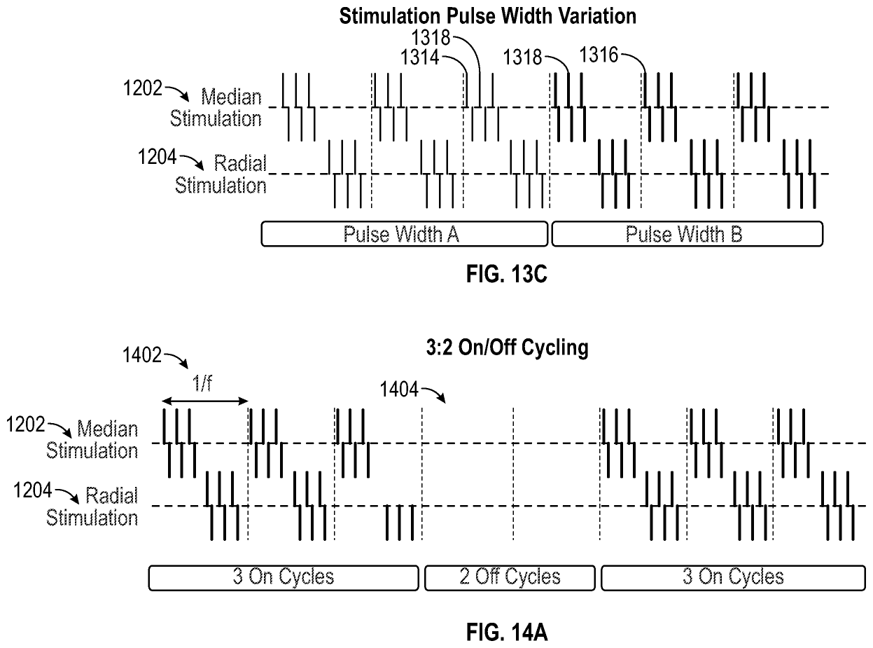

FIGS. 13A-C illustrate examples of how stimulation parameters (e.g., amplitude and pulse width) are varied between two or more prespecified values as stimulation is alternated across two nerves (e.g., median and radial nerve).

FIG. 13A illustrates an embodiment of the device 100 that delivers patterned stimulation to the median nerve 1202 and radial nerve 1204 where current amplitude is varied after a prespecified time period or prespecified number of bursts. As is illustrated in FIG. 13A, the current amplitude is initially current amplitude A with a value 1302. The current amplitude subsequently changes to current amplitude B with a different value 1304. In the illustrated embodiment, the value 1304 is greater than the value 1302 by an amount 1306. FIG. 13A is only exemplary and is not intended to limit the variations in current amplitude to the illustrated values or number of different amplitudes. Further, while FIG. 13A illustrates the variation occurring across multiple nerves (e.g., median and radial nerves), the disclosure is not so limited. The disclosed variations can be applied to only a single nerve.

The intensity of the electrical stimulation may vary from 0 mA to 500 mA, and a current may be approximately 1 to 11 mA in some cases. The electrical stimulation can be adjusted in different patients and with different methods of electrical stimulation. The increment of intensity adjustment may be, for example, 0.1 mA to 1.0 mA.

FIG. 13B illustrates an embodiment of the device 100 that delivers patterned stimulation to the median nerve 1202 and the radial nerve 1204 where current amplitude is ramped up during a burst, after a prespecified time period, or after a prespecified number of bursts. As is illustrated in FIG. 13B, the current amplitude is initially current amplitude A with a value 1308. The current amplitude subsequently changes to current amplitude B with a different value 1310. In the illustrated embodiment, the value 1308 is greater than the value 1310 by an amount 1312.

In some embodiments, the device 100 delivers patterned stimulation to the median nerve 1202 and the radial nerve 1204 where current amplitude is ramped down during a burst, after a prespecified time period, or after a prespecified number of bursts. For example, the patterned stimulation can initially have the current amplitude B with the value 1310. The current amplitude can subsequently ramp down and change to current amplitude A with the value 1308.

In some embodiments, the device 100 delivers patterned stimulation to the median nerve 1202 and the radial nerve 1204 where current amplitude ramps up and then down during a burst, after a prespecified time period, or after a prespecified number of bursts. For example, the patterned stimulation can initially have the current amplitude A with the value 1308. The current amplitude can subsequently ramp up to current amplitude B with the value 1310 followed by ramping down to current amplitude A with the value 1308.

In some embodiments, the device 100 delivers patterned stimulation to the median nerve 1202 and the radial nerve 1204 where current amplitude ramps down and then up during a burst, after a prespecified time period, or after a prespecified number of bursts. For example, the patterned stimulation can initially have the current amplitude B with the value 1310. The current amplitude can subsequently ramp down to current amplitude A with the value 1308 followed by ramping up to current amplitude B with the value 1310.

In some embodiments, the device 100 delivers patterned stimulation to the median nerve 1202 and the radial nerve 1204 where the amplitude of each pulse within a burst is randomly selected. For example, in some embodiments, the device 100 selects the amplitude of each pulse from a uniform distribution or normal (Gaussian) distribution, limited between amplitudes A and B.

In some embodiments, the value 1308 corresponds to the patient's minimum sensory threshold or paresthesia threshold (lower bound) and the value 1310 corresponds to a maximum comfortable threshold (upper bound). For example, during device 100 calibration, the patient may select the preset values 1308, 1310 for minimum sensory and comfortable thresholds which would set the lower and upper bounds for the waveform, respectively. In some embodiments, amplitude adjustments made during a treatment session would increase/decrease the lower and upper bound values 1308, 1310 together.

In some embodiments, higher stimulation amplitudes may activate smaller and more distant neural fibers in the median and radial nerves. Dynamically changing the amplitude as illustrated in FIG. 13B within a burst can generate asynchronous and stochastic activation that is distributed across nerve fibers. This activation may enhance the therapeutic mechanism of action by increasing desynchronization in downstream brain targets. The dynamic changes in amplitude may also generate more natural-feeling paresthesia sensations.

In some embodiments, the range of the values 1308, 1310 includes a minimum set to a stimulation amplitude at a pre-specified increment below an individual's minimum sensory threshold (sub-sensory) and a maximum set to the stimulation amplitude at an individual's maximum comfort level. The intensity of the electrical stimulation may vary from 0 mA to 500 mA, and a current may be approximately 1 to 11 mA in some cases. The electrical stimulation can be adjusted in different patients and with different methods of electrical stimulation. In some embodiments, the ramp rate comprises 0.001-100 mA/s (e.g., 0.001-0.01, 0.01-0.1, 0.1-1, 1-100 mA/s, and overlapping ranges therein). In some embodiments, the ramp rate is 0.001-100 mA/s. FIG. 13B is only exemplary and is not intended to limit the values 1308, 1310 and current ramp amplitudes to the illustrated values or number of different amplitudes. Further, while FIG. 13B illustrates the variation occurring across multiple nerves (e.g., median and radial nerves), the disclosure is not so limited. The disclosed variations can be applied to only a single nerve.

FIG. 13C illustrates an embodiment of the device 100 that delivers patterned stimulation to the median nerve 1202 and radial nerve 1204 where pulse width is varied after a prespecified time period or prespecified number of bursts. As is illustrated in FIG. 13C, the pulse width is initially pulse width A with a value 1314. The pulse width subsequently changes to pulse width B with a different value 1316. As is illustrated by a comparison between 1318 for pulse widths A and B, the value 1316 is greater than the value 1314. Of course, the subsequent value 1316 could be less than value 1318 in other embodiments. FIG. 13C is only exemplary and is not intended to limit the variations in pulse width to the illustrated values or number of different pulses. Further, while FIG. 13C illustrates the variation occurring across multiple nerves (e.g., median and radial nerves), the disclosure is not so limited. The disclosed variations can be applied to only a single nerve. A pulse width may range from, in some cases, 50 to 500 μs (micro-seconds), such as approximately 300 μs.

The pulse width of electrical stimulation applied to a peripheral nerve or neuron can be one factor that determines the number and types of nerves or neurons activated with each stimulation pulse. More specifically, varying pulse width applied to a peripheral nerve could advantageously produce a more pronounced desynchronization effect in activated brain region, including but not limited to thalamus, as this can vary the size of the neuronal sub-populations that are recruited during peripheral nerve stimulation. For example, an electrical stimulation pulse train with a fixed pulse width will recruit the same set of neurons, nerves, or nerve fibers with each pulse, which is not a natural characteristic of neuronal activity. In contrast, natural stimuli to the nervous system generate action potentials in a more probabilistic and stochastic fashion. In some embodiments, varying stimulation pulse width over time could be used to activate distinct neuronal populations with each pulse, which could more closely resemble physiological neural signaling. Varying pulse width can produce more natural sensations with stimulation of the median, radial, and ulnar nerves using implanted nerve cuffs in patients with upper limb amputation, and equally or more comfortable sensations with spinal cord stimulation for treatment of neuropathic pain. In some embodiments, pulse width could be varied between sensory threshold and maximum comfortable threshold for an individual, with stimulation amplitude (also referred to as current level or voltage level) held constant. Pulse width of transcutaneously applied electrical stimulation affects comfort and perceived sensation, so ranges can be determined based on feedback of an individual user.

In an alternative embodiment, the pulse width can be varied between a minimum and maximum set for each individual, where the minimum value is, for example, from about, at least about, or no more than about one of 100, 150, 200, 250, 300, or 350 microseconds and the maximum value is set based on an individual's comfort level at a fixed stimulation amplitude, and the rate of variation is restricted to (e.g., consists essentially of or comprises) 0.01-10,000 microseconds per second. In a further embodiment, the fixed stimulation amplitude is based on an individual's sensory level with a fixed pulse width in a range, for example, of between 100-500 microseconds (e.g., 100-250 microseconds, 250-500 microseconds, and overlapping ranges therein).

In alternative embodiments, stimulation amplitude is varied while pulse width is kept constant. In some embodiments, variation of stimulation amplitude (also referred to as current or voltage level, or current or voltage amplitude) can activate distinct neuronal populations with each pulse. In a further embodiment, the range of stimulation amplitude variation is restricted to (e.g., consists essentially of or comprises) a minimum set to the stimulation amplitude at an individual's minimum sensory threshold and a maximum set to the stimulation amplitude at an individual's maximum comfort level. In another embodiment, the minimum is set to a stimulation amplitude at a pre-specified increment below an individual's minimum sensory threshold (sub-sensory) and a maximum set to the stimulation amplitude at an individual's maximum comfort level wherein the pre-specified increment is, for example, about, at least about, or no more than about one of 0.1, 0.2, 0.25, 0.3, 0.4, 0.5, 0.6, 0.7, 0.75, 0.8, 0.9 or 1 mA. In some embodiments, the rate of change of the stimulation amplitude parameter may be between 0.001-100 mA/s.

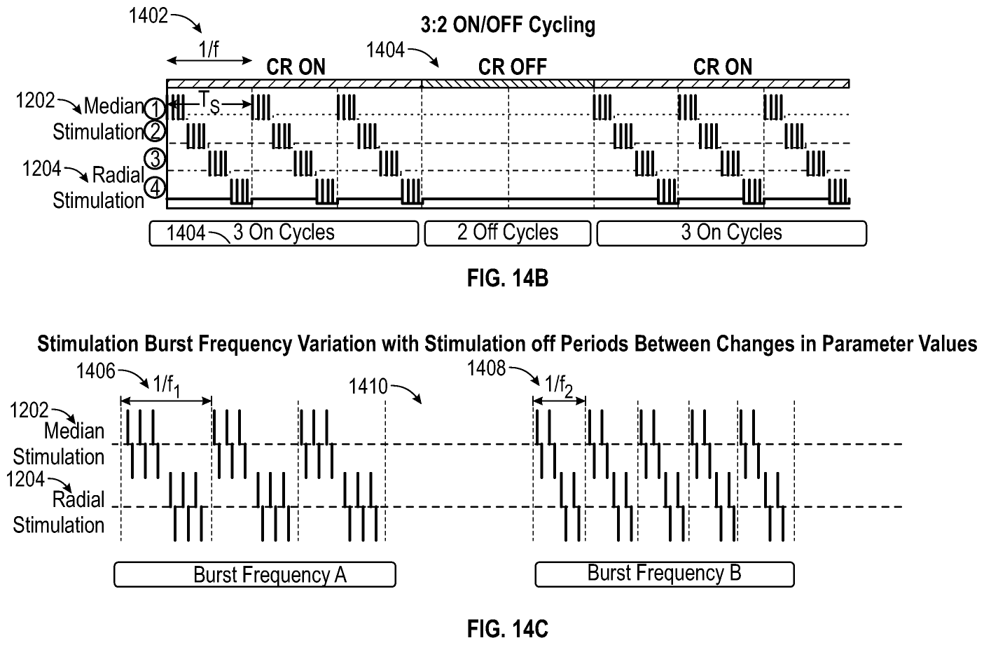

FIGS. 14A-C illustrate multiple examples of stimulation patterns with prespecified on/off periods as stimulation is alternated across multiple nerves (e.g., median and radial nerve). The plots show patterns of current delivered by the device 100 over time. FIGS. 14A and 14B illustrate embodiments of the device 100 that deliver patterned stimulation to multiple nerves where stimulation is delivered for three bursts 1402 (i.e., on period) and stimulation is not delivered for two bursts 1404 (i.e., off period). Of course, the disclosure is not so limited. In some embodiments, the number of on periods need not be three and instead can be any number (e.g., 1-10). Similarly, the number of off periods need not be two and instead can be any number (e.g., 1-10). In some embodiments as is Illustrated in FIGS. 14A and 14B, the stimulation pattern of on and off periods repeats. However, in some other embodiments, the stimulation pattern of on and off periods dynamically changes over time. In some embodiments, the on-cycle portion and the off-cycle portion are configured to maximize shifting of pathological neural oscillations away from their peak resonance frequency and/or enhancing spike-timing-dependent plasticity (STDP).

The embodiment illustrated in FIG. 14A delivers patterned stimulation to the median and radial nerves while the embodiment illustrated in FIG. 14B delivers patterned stimulation to more than two targeted nerves. In some embodiments, bursts can be defined by the period of the user's measured hand tremor as measured by motion sensors onboard the device 100. In certain embodiments, the period of the user's measured hand tremor corresponds to an initial burst pattern applied by the device 100. In certain embodiments, the device 100 subsequently varies the initial burst as shown in, for example, FIG. 14C. FIG. 14C illustrates a similar embodiment where burst frequency is varied between two or more prespecified values across on periods. For example, the device 100 in FIG. 14C delivers patterned stimulation to the median and radial nerves where stimulation is delivered at a burst frequency A having a period of 1/f₁ 1406 (i.e., on period) followed by an off period 1410. The device 100 then delivers stimulation at a burst frequency B having a period of 1/f₂ 1408.

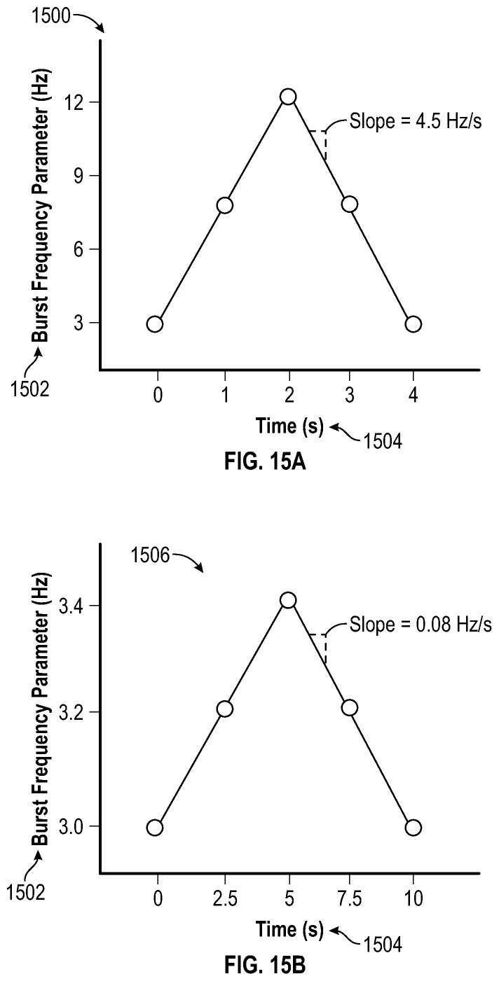

FIG. 15A illustrates an example of a ramping variation of the burst frequency parameter 1502 over time 1504. The burst frequency linearly ramps 1500 from 3 Hz to 12 Hz in time period of 2 seconds, which results in a rate of change of 4.5 Hz/s. FIG. 15B illustrates another example of a ramping variation of the burst frequency parameter 1502 over time 1504. The burst frequency linearly ramps 1506 from 3 Hz to 3.4 Hz in time period of 5 seconds, which results in a rate of change of 0.08 Hz/s. In some embodiments, one or more stimulation parameters could be varied as stimulation is applied to one or more target nerves or neurons, where stimulation parameters include burst frequency, pulse frequency, pulse width, on/off cycling, and stimulation amplitude. In some embodiments, variation can be performed as a sweep across a prespecified range of parameters (e.g., a linear ramp of values, an example of which is shown in FIGS. 15A and 15B, or sinusoidally-varying values).

In certain embodiments, a randomized or pseudo-randomized variation of parameters can be applied across a prespecified range of parameter values. In a further embodiment, variation of parameters can be distributed based on a predefined probabilistic distribution, including but not limited to a uniform, normal, Gaussian, chi square, binomial, or Poisson distribution. Alternatively, the probabilistic distribution function used to select the values for variation of parameters, such as burst frequency, can be set based on the observed tremor frequency distribution from multiple tremor-inducing task measurements. In some embodiments, there is a prespecified rate of changing parameters, which could theoretically range from changing a parameter value on a prespecified number of stimulation cycles, including but not limited to 1, 2, 3, 4, 5, 6, 7, 8, 9, 10, or more or less tremor cycles, or ranges including any two of the foregoing values (e.g., defined by burst or pulse frequency, non-limiting examples of which are shown in FIG. 12A and FIG. 12B, where the parameter value changes after every 3 stimulation cycles) up to the order of minutes. In some embodiments, this rate of parameter variation is selectable by the end user from a prespecified list of options. In other embodiments, the rate of parameter variation is set by the learning algorithm 222 based on some measured tremor characteristic, such as the rate of change in tremor frequency over time. The change in parameter values may occur instantaneously, or after a period in which stimulation is temporarily turned off for a duration between, for example, approximately 0.1 second and 10 seconds, as illustrated in FIGS. 14A and 14B.

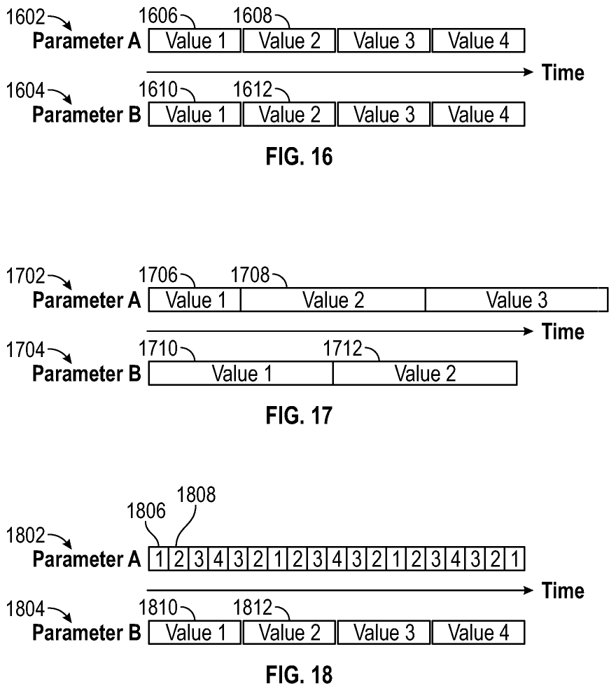

FIG. 16 illustrate an example of how multiple stimulation parameters (e.g., parameters A 1602 and B 1604) are simultaneously varied between two or more prespecified values as stimulation is applied to a nerve (e.g., median or radial nerve). As is illustrated in FIG. 16, parameter A 1602 has a value 1 1606 followed by a value 2 1608. Parameter B 1604 has a value 1 1610 followed by a value 2 1612. Parameter A 1602 and parameter B 1604 both change to their respective values 2 simultaneously. The values of parameter A 1602 and parameter B 1604 both further change to their respective values 3 simultaneously. Of course, the illustrated embodiment and values are only exemplary. In other embodiments, three or more stimulation patterns are simultaneously varied.

In certain embodiments, the method of varying multiple stimulation parameters in FIG. 16 is applied to at least one nerve. In other embodiments, the method for varying stimulation parameters in FIG. 16 is applied to multiple nerves. For example, parameters A 1602 and B 1604 can be varied for a first nerve (e.g., median nerve) accordingly to the method illustrated in FIG. 16 and for a second nerve (e.g., radial nerve) according to the method illustrated in FIG. 16. Of course, the values of the parameters for the first nerve need not be the same as the values of the parameters for the second nerve.

In certain embodiments, the same parameters (e.g., parameters A and B) are varied across at least two nerves. In other embodiments, the parameters varied across the first nerve according to the method illustrated in FIG. 16 are different from the parameters varied across the second nerve according to the method illustrated in FIG. 16.

In certain embodiments, the method of FIG. 16 can be implemented by alternating stimulation between multiple nerves with a specific burst frequency or used to stimulate a single nerve. In certain embodiments where multiple nerves are stimulated, the stimulation parameters can be varied for stimulation of the first nerve but may be fixed for stimulation of the second nerve.

In certain embodiments, the parameter values 1606-1612 disclosed in FIG. 16 can change over time. For example, in certain embodiments, the parameter values 1606-1612 change from therapy session to therapy session. As further explained below in certain embodiments, the parameter values 1606-1612 can be changed based on the learning algorithm 222 to optimize therapy. In certain embodiments, the parameter values are changed based on pre-session measures, such as tremor kinematic characteristics or system impedance.

FIG. 17 illustrate an example of how multiple stimulation parameters (e.g., parameters A 1702 and B 1704) are varied by alternately changing each parameter between two or more prespecified values as stimulation is applied to a nerve (e.g., median or radial nerve). As is illustrated in FIG. 17, parameter A 1702 has a value 1 1706 followed by a value 2 1708. Parameter B 1704 has a value 1 1710 followed by a value 2 1712. Parameter A 1702 and parameter B 1704 alternate changing their respective values. The values of parameter A 1702 and parameter B 1704 both alternate changing to their respective values 3. In certain embodiment, the values of parameter A 1702 and parameter B 1704 change asynchronously. Of course, the illustrated embodiment and values are only exemplary. In other embodiments, three or more stimulation patterns are alternately varied.

In certain embodiments, the method of varying multiple stimulation parameters in FIG. 17 is applied to at least one nerve. In other embodiments, the method for varying stimulation parameters in FIG. 17 is applied to multiple nerves. For example, parameters A 1702 and B 1704 can be varied for a first nerve (e.g., median nerve) accordingly to the method illustrated in FIG. 17 and for a second nerve (e.g., radial nerve) according to the method illustrated in FIG. 17. Of course, the values of the parameters for the first nerve need not be the same as the values of the parameters for the second nerve.

In certain embodiments, the same parameters (e.g., parameters A and B) are varied across at least two nerves. In other embodiments, the parameters varied across the first nerve according to the method illustrated in FIG. 17 are different from the parameters varied across the second nerve according to the method illustrated in FIG. 17.

In certain embodiments, the method of FIG. 17 can be implemented by alternating stimulation between multiple nerves with a specific burst frequency or used to stimulate a single nerve. In certain embodiments where multiple nerves are stimulated, the stimulation parameters can be varied for stimulation of the first nerve but may be fixed for stimulation of the second nerve.

In certain embodiments, the parameter values 1706-1712 disclosed in FIG. 17 can change over time. For example, in certain embodiments, the parameter values 1706-1712 change from therapy session to therapy session. As further explained below in certain embodiments, the parameter values 1706-1712 can be changed based on the learning algorithm 222 to optimize therapy. In certain embodiments, the parameter values are changed based on pre-session measures, such as tremor kinematic characteristics or system impedance.

FIG. 18 illustrate an example of how multiple stimulation parameters (e.g., parameters A and B) are varied by applying different timescales to each parameter as stimulation is applied to a nerve (e.g., median or radial nerve). As is illustrated in FIG. 18, parameter A 1802 has a value 1 1806 followed by a value 2 1808. Parameter B 1804 has a value 1 1810 followed by a value 2 1812. Parameter A 1802 and parameter B 1804 change their respective values based on different timescales. The values of parameter A 1802 and parameter B 1804 both change based on their respective timescale. Of course, the illustrated embodiment and values are only exemplary. In other embodiments, three or more stimulation patterns are alternately varied.

In certain embodiments, parameter A 1802 (e.g., stimulation amplitude, pulse width) and parameter B 1804 (e.g., burst frequency, pulse frequency) are varied on different timescales. For example, in certain embodiments, parameter A 1802 may be varied pulse-to-pulse (every few tens of milliseconds or hundreds of milliseconds), whereas parameter B 1804 may be varied on a time scale of seconds to minutes.

In certain embodiments, the method of varying multiple stimulation parameters in FIG. 18 is applied to at least one nerve. In other embodiments, the method for varying stimulation parameters in FIG. 18 is applied to multiple nerves. For example, parameters A 1802 and B 1804 can be varied for a first nerve (e.g., median nerve) accordingly to the method illustrated in FIG. 18 and for a second nerve (e.g., radial nerve) according to the method illustrated in FIG. 18. Of course, the values of the parameters for the first nerve need not be the same as the values of the parameters for the second nerve.

In certain embodiments, the same parameters (e.g., parameters A and B) are varied across at least two nerves. In other embodiments, the parameters varied across the first nerve according to the method illustrated in FIG. 18 are different from the parameters varied across the second nerve according to the method illustrated in FIG. 18.

In certain embodiments, the method of FIG. 18 can be implemented with different timescales for multiple nerves with a specific burst frequency or used to stimulate a single nerve. In certain embodiments where multiple nerves are stimulated, the stimulation parameters can be varied for stimulation of the first nerve but may be fixed for stimulation of the second nerve.

In certain embodiments, the parameter values 1806-1812 disclosed in FIG. 18 can change over time. For example, in certain embodiments, the parameter values 1806-1812 change from therapy session to therapy session. As further explained below in certain embodiments, the parameter values 1806-1812 can be changed based on the learning algorithm 222 to optimize therapy. In certain embodiments, the parameter values are changed based on pre-session measures, such as tremor kinematic characteristics or system impedance.

In certain embodiments, different methods (e.g., methods disclosed in FIGS. 16-18) for varying multiple parameters can be used for different therapy session or during the same therapy session. For example, in certain embodiments, simultaneous variation of parameters as disclosed in FIG. 16 can be used for a first time frame (e.g., 5 minutes) of the therapy session, followed by a braided variation (FIG. 17) for a second time frame (e.g., next 5 minutes) of the therapy session. In certain embodiments, the values or first set of parameters varied during a first time frame are followed by a second set of parameters which are varied during a second time frame.

In certain embodiments, adaptive learning via the learning algorithm 222 is employed in combination with any of the methods illustrated in FIGS. 16-18. In certain embodiments, the learning algorithm 222 uses active and/or passive kinematic measurements during or after stimulation sessions to assess how stimulation parameter changes impact real-time therapeutic outcomes (e.g., tremor improvements). For example, if specific parameter values produce greater therapeutic outcomes than other values, then the stimulation method is modified during the same session to only use the corresponding parameter values.

In certain embodiments, the learning algorithm 222 uses satisfaction data during or after stimulation sessions to assess how stimulation parameter changes impact real-time therapeutic outcomes (e.g., tremor improvements). For example, if specific parameter values produce greater therapeutic outcomes than other values, then the stimulation method is modified during the same session to only use the corresponding parameter values.

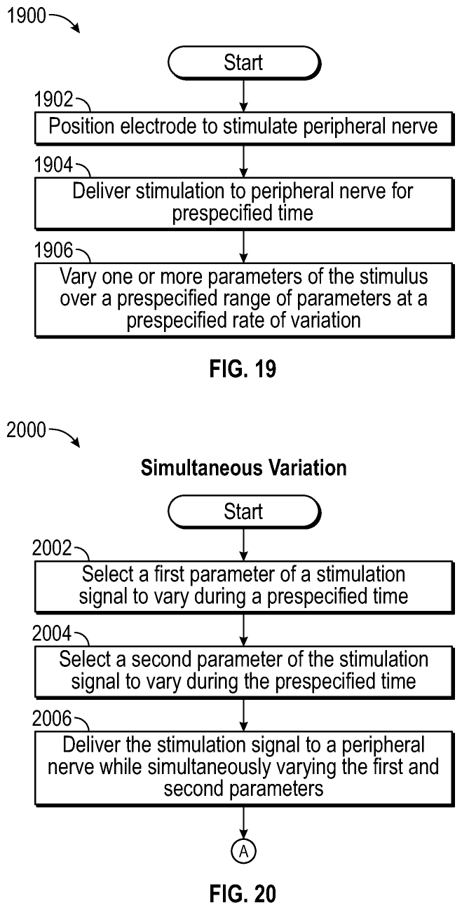

FIG. 19 illustrates a flow chart of an embodiment of a process 1900 for varying one or more parameters of a stimulus over a prespecified range of parameters at a prespecified rate of variation. The process 1900 can be implemented by any of the systems discussed above. The process 1900 can be implemented alone or in combination with other processes described herein.

In several embodiments, the process 1900 can begin at block 1902 where the electrode 102 is positioned to stimulate a peripheral nerve. In some instances, the electrode 102 is a component of the device 100. The method moves to block 1904 where the device 100 delivers stimulation to the peripheral nerve for a prespecified time. The method then moves to block 1906 where one or more parameters of the stimulus are varied over a prespecified range of parameter values. In certain embodiments, the one or more parameters are further varied over a prespecified rate of variation.

Variability can be applied to one or more of the following parameters for stimulating a nerve including but not limited to burst frequency or alternating frequency, pulse frequency, pulse width, pulse spacing, intensity, current amplitude, voltage amplitude, duration of stimulation, on/off periods, or amplitude envelope periods. Variability can be applied across multiple stimulation parameters for stimulating a nerve including but not limited to simultaneous variation, braided variation, timescale variation, and adaptive learning. In certain embodiments, adaptive learning is employed in combination with the listed variations as well as other variations to improve outcomes.

FIG. 20 illustrates a flow chart of an embodiment of a process 2000 for simultaneously varying multiple stimulation parameters (e.g., parameters A and B) between two or more prespecified values as stimulation is applied to a nerve (e.g., median or radial nerve). The process 2000 can be implemented by any of the systems discussed above. The process 2000 can be implemented alone or in combination with other processes described below.

The process 2000 can begin at block 2002 with selecting a first parameter of a stimulation signal to vary during a prespecified time. Variability can be applied to one or more of the following parameters for stimulating a nerve including but not limited to burst frequency or alternating frequency, pulse frequency, pulse width, pulse spacing, intensity, current amplitude, voltage amplitude, duration of stimulation, on/off periods, or amplitude envelope periods. At block 2004, the method selects a second parameter of the stimulation signal to vary during a prespecified time.

The process moves to block 2006 where the stimulation signal is delivered while simultaneously varying the first and second parameters. The process 2000 can be applied to one or more nerves. For example, parameters A and B can be varied for a first nerve (e.g., median nerve) and for a second nerve (e.g., radial nerve). Of course, the values of the parameters for the first nerve need not be the same as the values of the parameters for the second nerve.

In certain embodiments, the same parameters (e.g., parameters A and B) are varied across at least two nerves. In other embodiments, the parameters varied across the first nerve are different from the parameters varied across the second nerve. In certain embodiments, the process 2000 can be implemented by alternating stimulation between multiple nerves with a specific burst frequency or used to stimulate a single nerve. In certain embodiments where multiple nerves are stimulated, the stimulation parameters can be varied for stimulation of the first nerve but may be fixed for stimulation of the second nerve.

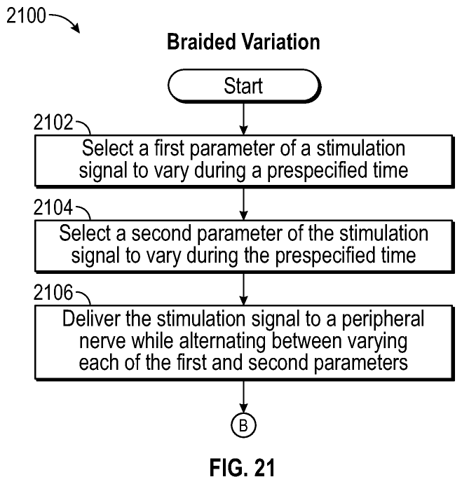

FIG. 21 illustrates a flow chart of an embodiment of a process 2100 for alternately varying multiple stimulation parameters (e.g., parameters A and B) between two or more prespecified values as stimulation is applied to a nerve (e.g., median or radial nerve). The process 2100 can be implemented by any of the systems discussed above. The process 2100 can be implemented alone or in combination with other processes described below.

The process 2100 can begin at block 2102 with selecting a first parameter of a stimulation signal to vary during a prespecified time. Variability can be applied to one or more of the following parameters for stimulating a nerve including but not limited to burst frequency or alternating frequency, pulse frequency, pulse width, pulse spacing, intensity, current amplitude, voltage amplitude, duration of stimulation, on/off periods, or amplitude envelope periods. At block 2104, the method selects a second parameter of the stimulation signal to vary during a prespecified time.

The process moves to block 2106 where the stimulation signal is delivered while alternating between varying each of the first and second parameters. The process 2100 can be applied to one or more nerves. For example, parameters A and B can be varied for a first nerve (e.g., median nerve) and for a second nerve (e.g., radial nerve). Of course, the values of the parameters for the first nerve need not be the same as the values of the parameters for the second nerve.

In certain embodiments, the same parameters (e.g., parameters A and B) are varied across at least two nerves. In other embodiments, the parameters varied across the first nerve are different from the parameters varied across the second nerve. In certain embodiments, the process 2100 can be implemented by alternating stimulation between multiple nerves with a specific burst frequency or used to stimulate a single nerve. In certain embodiments where multiple nerves are stimulated, the stimulation parameters can be varied for stimulation of the first nerve but may be fixed for stimulation of the second nerve.

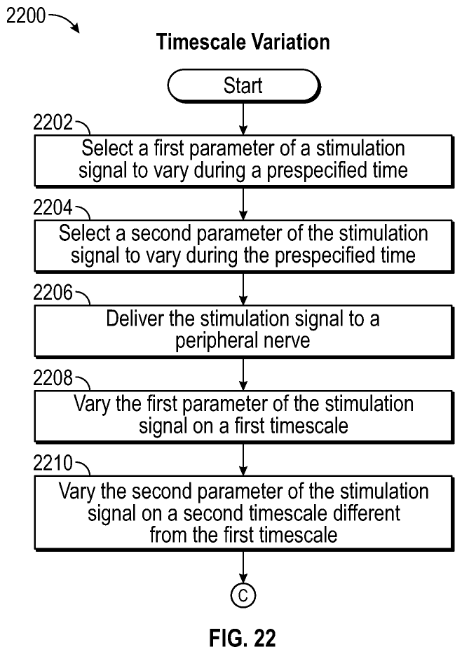

FIG. 22 illustrates a flow chart of an embodiment of a process for varying multiple stimulation parameters (e.g., parameters A and B) between two or more prespecified values by applying different timescales to each parameter as stimulation is applied to a nerve (e.g., median or radial nerve). The process 2200 can be implemented by any of the systems discussed above. The process 2200 can be implemented alone or in combination with other processes described below.

The process 2200 can begin at block 2202 with selecting a first parameter of a stimulation signal to vary during a prespecified time. Variability can be applied to one or more of the following parameters for stimulating a nerve including but not limited to burst frequency or alternating frequency, pulse frequency, pulse width, pulse spacing, intensity, current amplitude, voltage amplitude, duration of stimulation, on/off periods, or amplitude envelope periods. At block 2204, the method selects a second parameter of the stimulation signal to vary during a prespecified time.

The process moves to block 2206 where the stimulation signal is delivered to a peripheral nerve. While the stimulation signal is being delivered, the first parameter of the stimulation signal is varied on a first timescale at block 2208 and the second parameter of the stimulation signal is varied on a second timescale at block 2210. The process 2200 can be applied to one or more nerves. In this way, in certain embodiments, blocks 2206, 2208, and 2210 are performed concurrently.

In certain embodiments, parameters A and B can be varied for a first nerve (e.g., median nerve) and for a second nerve (e.g., radial nerve). Of course, the values of the parameters for the first nerve need not be the same as the values of the parameters for the second nerve.

In certain embodiments, the same parameters (e.g., parameters A and B) are varied across at least two nerves. In other embodiments, the parameters varied across the first nerve are different from the parameters varied across the second nerve. In certain embodiments, the process 2200 can be implemented by alternating stimulation between multiple nerves with a specific burst frequency or used to stimulate a single nerve. In certain embodiments where multiple nerves are stimulated, the stimulation parameters can be varied for stimulation of the first nerve but may be fixed for stimulation of the second nerve.

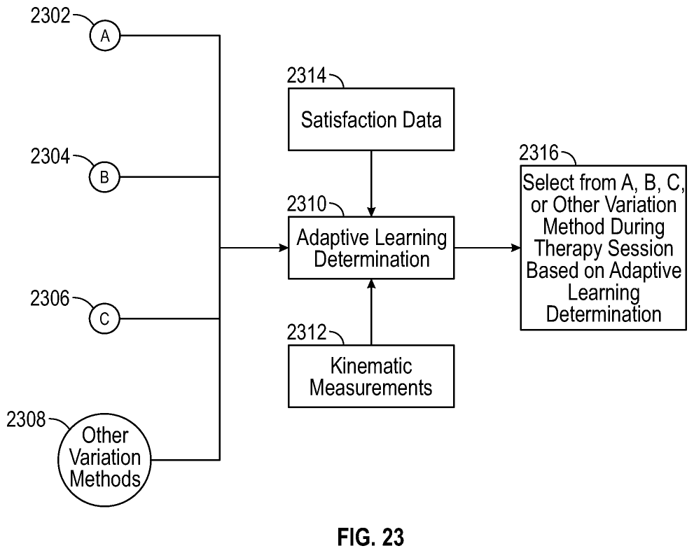

FIG. 23 illustrates an architecture 2300 for determining a method that varies multiple stimulation parameters based on adaptive learning. The architecture 2300 illustrated in FIG. 23 can be employed in combination with one or more of the processes 2000-2200 discussed above. In certain embodiments, the processes 2000-2200 correspond to blocks 2302, 2304, 2306 in FIG. 23, respectively. In certain embodiments, block 2308 can correspond to a method for varying stimulation patterns across a nerve that is not the same as the methods corresponding to blocks 2302, 2304, 2306. For example, the method associated with block 2308 can begin as one of the methods associated with blocks 2302-2306 but was subsequently adjusted or modified based on blocks 2312 and/or 2314.

The architecture 2300 further includes block 2310 where adaptive learning is employed to select a process from the processes 2302-2308 for use during a therapy session at block 2316. In certain embodiments, the adaptive learning determination 2310 is performed by the learning algorithm 222. The learning algorithm 222 can include programmed instructions for performing processes as discussed herein for detection of input conditions, processing data, and control of output conditions. The learning algorithm 222 can be executed by the one or more hardware processors of the neuromodulation (e.g., neurostimulation) device 100 alone or in combination with the base station 150, the user interface device 150, and/or the cloud 122.

At block 2310, the adaptive learning determination can leverage kinematic measurements 2312 as well as satisfaction data 2314. The kinematic measurements 2312 can include but is not limited to accelerometer or gyroscope data from the sensors 112, 207 (e.g., IMU). In certain embodiments, the kinematic measurements 2312 can include test kinematic data taken during a therapy session. In certain embodiments, the kinematic measurements 2312 can include passive kinematic data. Passive kinematic data is data collected at times outside of the therapy session.

In some embodiments, the neuromodulation, e.g., neurostimulation device 100 or the user interface device 150 with sensors can collect kinematic measurements 2312 (test and/or passive data), or data from other sensors, can measure data over a longer period of time, for example 1, 2, 3, 4, 5, 10, 20, 30 weeks, 1, 2, 3, 6, 9, 12 months, or 1, 2, 3, 5, 10 years or more or less, or ranges incorporating any two of the foregoing values, to determine features, or biomarkers, associated with the onset of tremor diseases, such as essential tremor, Parkinson's disease, dystonia, multiple sclerosis, Lyme disease, etc. Biomarkers could include specific changes in one or more features of the data over time, or one or more features crossing a predetermined threshold. In some embodiments, features of tremor inducing tasks have been stored on the neurostimulation device 100 and used to automatically activate sensors when those tremor inducing tasks are being performed, to measure and store data to memory during relevant times.

The devices, systems and methods described above and in the claims are used, in several embodiments can be used for the treatment of depression (including but not limited to post-partum depression, depression affiliated with neurological diseases, major depression, seasonal affective disorder, depressive disorders, etc.), inflammation (e.g., neuroinflammation), Lyme disease, stroke, neurological diseases (such as Parkinson's and Alzheimer's), and gastrointestinal issues (including those in Parkinson's disease). The devices described herein may also be used for the treatment of inflammatory bowel disease (such as Crohn's disease, colitis, and functional dyspepsia), rheumatoid arthritis, multiple sclerosis, psoriatic arthritis, osteoarthritis, psoriasis and other inflammatory diseases. The devices described herein can be used for the treatment of inflammatory skin conditions in some embodiments. The neuromodulation devices, e.g., neurostimulation devices, described herein can be used for the treatment of chronic fatigue syndrome. The devices described herein can be used for the treatment of chronic inflammatory symptoms and flare ups. Bradykinesia, dyskinesia, rigidity may also be treated according to several embodiments. In several embodiments, rehabilitation as a result of certain events is treated, for example, rehabilitation from stroke or other cardiovascular events. In several embodiments, treatment of involuntary and/or repetitive movements is provided, including but not limited to tics, twitches, etc. (including, for example, Tourette Syndrome, tic disorders). Rhythmic and non-rhythmic involuntary movements may be controlled in several embodiments. Involuntary vocal tics and other vocalizations may also be treated. Systems and methods to reduce habituation and/or tolerance to stimulation in the disorders and symptoms identified herein are provided in several embodiments by, for example, introducing variability in stimulation parameter(s) described herein. Habituation and/or tolerance to neurostimulation that occur in the treatment of movement, inflammatory, neurological and psychiatric disorders are treated in several embodiments.

The satisfaction data 2314 can include but is not limited to subjective data provided by the user. The subjective data can relate to pre or post treatment and/or patient activities of daily living (ADL). In certain embodiments, the patient inputs a value that reflects a level of satisfaction. The level of satisfaction can be selected from a predetermined range. In certain embodiments, the range is from 1 to 4. Of course, the range can be any range and is not limited to 1 to 4. For example, the user can provide input to the user interface 212 in the form of a patient session impression of improvement (PSII) score and/or a patient satisfaction scope.

In certain embodiments at block 2310, the learning algorithm 222 determines a level of patient therapeutic benefit based on the passive kinematic measurements 2312 without requiring the patient to input a subjective satisfaction level. In certain embodiments, the learning algorithm 222 receives the kinematic measurements 2312 measured during the therapy session along with satisfaction data 2314 input by the user. In this way in certain embodiments, the learning algorithm 222 can determine a level of patient therapeutic benefit based on both the passive kinematic data and the patient provided subjective satisfaction level.

At block 2310, the learning algorithm 222 can select from processes 2302-2308 for varying parameter(s) employed during therapy session based on adaptive learning to improve tremor therapeutic treatment. In certain embodiments, the learning algorithm 222 can select from a plurality of stimulation parameters (e.g., burst frequency and pulse frequency) to vary one parameter across one or more nerves (e.g., median and/or radial nerve) and/or select multiple stimulation parameters to vary across one or more nerves.

In certain embodiments, the plurality of stimulation parameters accessed by the learning algorithm 222 can be a subset of all of the stimulation parameters and or patterns of applying stimulation parameters. For example, in certain embodiments, the learning algorithm 222 selects from the processes 2302-2308 for which a positive outcome is predicted by the learning algorithm 222. In certain embodiments, the learning algorithm 222 modifies the one or more parameters of the selected process based on the individual patient to further personalize the stimulation parameters. In certain embodiments, the learning algorithm 222 can automatically determine a correlation between the satisfaction data 2314 and/or the kinematic measurements 2312 and neurostimulation therapy outcomes to select from the processes 2302-2308.

In some embodiments, disorders and symptoms caused or exacerbated by microbial infections (e.g., bacteria, viruses, fungi, and parasites) are treated. Symptoms include but are not limited to sympathetic/parasympathetic imbalance, autonomic dysfunction, inflammation, motor and balance dysfunction, pain and other neurological symptoms. Disorders include but are not limited to tetanus, meningitis, Lyme disease, urinary tract infection, mononucleosis, chronic fatigue syndrome, autoimmune disorders, etc. In some embodiments, autoimmune disorders and/or pain unrelated to microbial infection is treated, including for example, inflammation, headache, back pain, joint pain and stiffness, muscle pain and tension, etc.

In several embodiments, the neuromodulation, e.g., neurostimulation, devices described herein can be used for the treatment of cardiac conditions (such as atrial fibrillation, hypertension, and stroke) and for the treatment of immune dysfunction. Epilepsy is treated in one embodiment. The devices described herein can be used to stimulate the autonomic nervous system. The devices described herein can be used to balance the sympathetic/parasympathetic nervous systems.

In some embodiments, provided herein are treatments of restless leg syndrome, periodic limb movement disorder, repetitive movements of the limbs and abnormal sensation. Devices described herein can be placed, for example, on the wrist or leg (or both) to treat limb disorders. In some embodiments, vagus nerve stimulation is used to treat restless leg syndrome, periodic limb movement disorder, repetitive movements of the limbs and/or abnormal limb sensation. With respect to the leg, a device may be placed, for example, on the thigh, calf, ankle or other location suitable to treat the target nerve(s). The devices described herein can be used to stimulate the autonomic nervous system. The devices described herein can be used to balance the sympathetic/parasympathetic nervous systems. Dysfunction or imbalance of the autonomic nervous system is believed to be a potential underlying mechanism for various chronic diseases. Autonomic dysfunction can develop when the nerves of the ANS are damaged or degraded or without any known neural pathology. This condition is called autonomic neuropathy or dysautonomia. Autonomic dysfunction can range from mild to life-threatening and can affect part of the ANS or the entire ANS. Sometimes the conditions that cause problems are temporary and reversible. Others are chronic, or long term, and may continue to worsen over time. Examples of chronic diseases that are associated with autonomic dysfunction include, but are not limited to, diabetes, Parkinson's disease, tremor, cardiac arrhythmias including atrial fibrillation, hypertension, overactive bladder, urinary incontinence, fecal incontinence, inflammatory bowel diseases, rheumatoid arthritis, migraine, depression, and anxiety.

In some embodiments, disorders and symptoms caused or exacerbated by microbial infections (e.g., bacteria, viruses, fungi, and parasites) are treated. Symptoms include but are not limited to sympathetic/parasympathetic imbalance, autonomic dysfunction, inflammation (including but not limited to neuroinflammation and other inflammation), motor and balance dysfunction, pain and other neurological symptoms. Disorders include but are not limited to tetanus, meningitis, Lyme disease, urinary tract infection, mononucleosis, chronic fatigue syndrome, autoimmune disorders, etc. In some embodiments, autoimmune disorders and/or pain unrelated to microbial infection is treated, including for example, inflammation (e.g., neuroinflammation, etc.), headache, back pain, joint pain and stiffness, muscle pain and tension, etc. Other disorders (e.g., hypertension, dexterity, and cardiac dysrhythmias) can also be treated using the embodiments described herein.

In some embodiments, modulation of the blood vessel (either dilation or constriction) is provided using the devices and methods described herein (e.g., through nerve stimulation). Such therapy may, in turn, reduce inflammation (including but not limited to inflammation post microbial infection). The devices and methods described herein increase, decrease or otherwise balance vasodilation and vasoconstriction through neuromodulation in some embodiments. For example, reduction of vasodilation is provided in several embodiments to treat or prevent migraine or other conditions that are aggravated by vasodilation. In other embodiments, vasoconstriction is reduced in, for example, conditions in which dilation is beneficial (such as with high blood pressure and pain). In one embodiment, reduction in inflammation treats tinnitus. In some embodiments, modulation of the blood vessel (either dilation or constriction) is used to treat tinnitus. Tinnitus may be treated according to several embodiments through modulation (e.g., stimulation) of the vagus nerve alone or in conjunction with one, two or more other nerves (including for example the trigeminal nerve, great auricular nerve, nerves of the auricular branch, auricular branch of the vagus nerve, facial nerve, the auriculotemporal nerve, etc.). In one embodiment, nerves other than the vagus nerve are modulated to treat tinnitus. Cranial/auditory nerves may be modulated to treat tinnitus and/or auricular inflammation in some embodiments. Auricular devices may be used in conjunction with devices placed on limbs to in some embodiments (e.g., an ear device along with a wrist device).

Any of the neuromodulation devices discussed herein can be utilized to modulate (e.g., stimulate) median, radial, ulnar, sural, femoral, peroneal, saphenous, tibial and/or other nerves or meridians accessible on the limbs of a subject alone or in combination with a one or more other nerves (e.g., vagal nerve) in the subject, for example, via a separate neuromodulation device. In some embodiments, provided herein are treatments of restless leg syndrome, periodic limb movement disorder, repetitive movements of the limbs and abnormal sensation. Treatment of movement disorders herein also includes, for example, treatment of involuntary and/or repetitive movements, such as tics, twitches, etc. (including, but not limited to, Tourette Syndrome, tic disorders for example). Rhythmic and/or non-rhythmic involuntary movements may be controlled in several embodiments. Involuntary vocal tics and other vocalizations may also be treated. Devices described herein can be placed, for example, on the wrist or leg (or both) to treat limb disorders. In some embodiments, vagus nerve stimulation is used to treat restless leg syndrome, periodic limb movement disorder, repetitive movements of the limbs and/or abnormal limb sensation. The vagus nerve may be stimulated alone or in addition to one or more of the sural, femoral, peroneal, saphenous, and tibial nerves. Alternatively, one or more of the sural, femoral, peroneal, saphenous, and tibial nerves are stimulated without stimulating the vagus nerve.

In some embodiments, transcutaneous nerve neuromodulation at the arm and/or wrist (e.g., median and/or radial nerve stimulation) can advantageously inhibit sympathoexcitatory related increases in blood pressure and premotor sympathetic neural firing in the rostral ventrolateral medulla (rVLM). Neuromodulation of the median and/or radial nerves, for example, can provide more convergent input into cardiovascular premotor sympathetic neurons in the rVLM.

Also, in some embodiments, vagal nerve stimulation can modulate the trigeminal nuclei to inhibit inflammation. Thus, in several embodiments the vagal nerve is stimulated to reduce inflammation via a trigeminal pathway. In other embodiments, the trigeminal nerve is stimulated directly instead of or in addition to the vagus nerve. In some embodiments, transcutaneous nerve stimulation projects to the nucleus tractus solitarii (NTS) and spinal trigeminal nucleus (Sp5) regions to modulate trigeminal sensory complex excitability and connectivity with higher brain structures. Trigeminal sensory nuclei can be involved in neurogenic inflammation during migraine (e.g., characterized by vasodilation). In some embodiments, stimulation of the nerve modulates the trigeminal sensory pathway to ameliorate migraine pathophysiology and reduce headache frequency and severity. For example, increased activation of raphe nuclei and locus coeruleus may inhibit nociceptive processing in the sensory trigeminal nucleus. Human skin is well innervated with autonomic nerves and neuromodulation (e.g., stimulation) of nerve or meridian points as disclosed herein can potentially help in treatment of migraine or other headache conditions. For example, transcutaneous nerve stimulation of afferent nerves in the periphery or distal limbs, including but not limited to median nerve, are connected by neural circuits to the arcuate nucleus of the hypothalamus. In some embodiments, the devices and methods described herein increase, decrease or otherwise balance vasodilation and vasoconstriction through neuromodulation (such as the vagus nerve, trigeminal nerve and/or other nerves surrounding the ear). For example, reduction of vasodilation is provided in several embodiments to treat or prevent migraine or other conditions that are exacerbated by vasodilation. In other embodiments, vasoconstriction is reduced in, for example, conditions in which dilation is beneficial (such as with high blood pressure and pain). In some embodiments, modulation of the blood vessel (either dilation or constriction) is used to treat tinnitus. In one embodiment, the devices and methods described herein reduce inflammation (including but not limited to inflammation post microbial infection), and the reduction in inflammation treats tinnitus.

### Terminology

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under”, or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps. Embodiments described herein may also consist or consist essentially of the features or steps disclosed in connection with such embodiments.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description. The methods disclosed herein include certain actions taken by a practitioner; however, they can also include any third-party instruction of those actions, either expressly or by implication. For example, actions such as “percutaneously stimulating an afferent peripheral nerve” includes “instructing the stimulation of an afferent peripheral nerve.”

1-199. (canceled)

200. A device comprising:
a band;
a first stimulating electrode to stimulate a first nerve;
a first set of common electrodes;
wherein one or more of the first set of common electrodes is positioned on each opposite side of the first stimulating electrode on the band;
wherein the first stimulating electrode and the first set of common electrodes create a first stimulation current path configured to activate the first nerve during a first time frame;
a second stimulating electrode to stimulate a second nerve;
a second set of common electrodes;
wherein one or more of the second set of common electrodes is positioned on each opposite side of the second stimulating electrode on the band;
wherein the second stimulating electrode and the second set of common electrodes create a second stimulation current path configured to activate the second nerve during a second time frame;
wherein during the second time frame, the device can maintain or change a state of one or more of any of the stimulating electrodes and/or one or more of any of the common electrodes; and
wherein one or more of the stimulation current paths and/or a number of stimulation current paths created by the device during the first time frame is configured to change during the second time frame.

201. The device of claim 200, wherein the device is configured to change a parameter of a stimulation signal sent to one or more of any of the stimulating electrodes between the first and second time frames to create one or more stimulation channels.

202. The device of claim 201, wherein the parameter of the stimulation signal can include a stimulation amplitude, a frequency, a patterned burst stimulation, an interval, a time of day, an individual session or a cumulative on time.

203. The device of claim 200, wherein the device is configured to activate different stimulation current paths during the first time frame as compared to the second time frame.

204. The device of claim 200, wherein the device is configured to change one or more parameters of a stimulation signal sent by the first stimulating electrode and/or the second stimulating electrode during the first time frame as compared to the second time frame.

205. The device of claim 201, wherein the parameter comprises a pattern, wherein the pattern comprises a burst stimulation pattern.

206. The device of claim 201, wherein the parameter comprises a stimulation amplitude.

207. The device of claim 201, wherein the parameter comprises a stimulation frequency.

208. The device of claim 200, wherein the device is configured to activate the first set of common electrodes to create multiple stimulation current paths between the first stimulating electrode and the first set of common electrodes.

209. The device of claim 200, wherein the device is configured to activate the second set of common electrodes to create multiple stimulation current paths between the second stimulating electrode and the second set of common electrodes.

210. The device of claim 200, wherein the device is configured to activate the first set of common electrodes while the second set of common electrodes is deactivated, and wherein the device activates the second set of common electrodes while the first set of common electrodes is deactivated.

211. The device of claim 200, wherein a level of activation of the first nerve or the second nerve caused by the stimulation current paths varies depending on a proximity of the first nerve or the second nerve to the stimulation current paths.

212. The device of claim 200, wherein the first stimulating electrode and the first set of common electrodes include at least three electrodes positioned transcutaneously to target a median nerve.

213. The device of claim 200, wherein the second stimulating electrode and the second set of common electrodes include at least three electrodes positioned transcutaneously to target a radial nerve.

214. The device of claim 200, wherein the first stimulating electrode, the first set of common electrodes, the second stimulating electrode, and the second set of common electrodes comprise dry electrodes.

215. A device comprising:
a band;
a first stimulating electrode to stimulate a first nerve;
a first common electrode;
wherein the first common electrode is positioned adjacent the first stimulating electrode on the band;
wherein the first stimulating electrode and the first common electrode create a first stimulation current path configured to activate the first nerve during a first time frame;
a second stimulating electrode to stimulate a second nerve;
a second common electrode;
wherein the second common electrode is positioned adjacent the first stimulating electrode on the band;
wherein the second stimulating electrode and the second common electrode create a second stimulation current path configured to activate the second nerve during a second time frame;
wherein during the second time frame, the device can maintain or change a state of one or more of any of the stimulating electrodes and/or one or more of any of the common electrodes; and
wherein one or more of the stimulation current paths and/or a number of stimulation current paths created by the device during the first time frame is configured to change during the second time frame.

216. The device of claim 215, wherein the device is configured to change a parameter of a stimulation signal sent to one or more of any of the stimulating electrodes between the first and second time frames to create one or more stimulation channels.

217. The device of claim 216, wherein the parameter of the stimulation signal can include a stimulation amplitude, a frequency, a patterned burst stimulation, an interval, a time of day, an individual session or a cumulative on time.

218. The device of claim 215, wherein the device is configured to change one or more parameters of a stimulation signal sent by the first stimulating electrode and/or the second stimulating electrode during the first time frame as compared to the second time frame.

219. The device of claim 215, wherein the device is configured to activate the first common electrode while the second common electrode is deactivated, and wherein the device activates the second common electrode while the first common electrode is deactivated.
